(19) United States
(12) Patent Application Publication
(10) Pub. No.: US 2017/0266323 A1
(43) Pub. Date: Sep. 21, 2017

(54) NEAR-INFRARED-II FLUORESCENT AGENTS, METHODS OF MAKING NEAR-INFRARED-II FLUORESCENT AGENTS, AND METHODS OF USING WATER-SOLUBLE NIR-II FLUORESCENT AGENTS

(71) Applicant: The Board of Trustees of the Leland Stanford Junior University, Palo Alto, CA (US)

(72) Inventors: Zhimin Tao, Highland Park, NJ (US); Guosong Hong, Stanford, CA (US); Yingping Zou, Changsha City (CN); Chihiro Fukunaga, Kawaguchi City (JP); Hongjie Dai, Cupertino, CA (US); Shuo Diao, Stanford, CA (US); Alex Antaris, Stanford, CA (US)

(21) Appl. No.: 15/610,959

(22) Filed: Jun. 1, 2017

Related U.S. Application Data
(63) Continuation of application No. 14/463,790, filed on Aug. 20, 2014.
(60) Provisional application No. 61/867,700, filed on Aug. 20, 2013.

(51) Int. Cl. A61K 49/00; G01N 33/553; A61B 5/00

(52) U.S. Cl. CPC A61K 49/0021; A61K 49/0082; G01N 33/553; A61B 5/0071

(57) ABSTRACT

Embodiments of the present disclosure provide for compositions including organic, water-soluble NIR-II fluorescent agent that emit radiation at about 1.0 to 1.7 μm, methods of making the composition, methods of imaging a disease and related biological events, methods of imaging, monitoring and/or assessing a disease and related biological events, and the like.

### CROSS-REFERENCE TO RELATED APPLICATION

This application is a continuation of co-pending U.S. application entitled “NEAR-INFRARED-II FLUORESCENT AGENTS, METHODS OF MAKING NEAR-INFRARED-II FLUORESCENT AGENTS, AND METHODS OF USING WATER-SOLUBLE NIR-II FLUORESCENT AGENTS” having Ser. No. 14/463,790, filed on Aug. 20, 2014, which application claims priority to U.S. Provisional application entitled “NEAR-INFRARED-II FLUORESCENT AGENTS, METHODS OF MAKING NEAR-INFRARED-II FLUORESCENT AGENTS, AND METHODS OF USING WATER-SOLUBLE NIR-II FLUORESCENT AGENTS” having Ser. No. 61/867,700, filed on Aug. 20, 2013, both of which are incorporated herein by reference.

### BACKGROUND

Traditional near-infrared (NIR) imaging in the 750-800 nm region has been widely pursued for biological research and biomedical applications in the past decades. It is well known NIR imaging can benefit from reduced optical absorption and lower autofluorescence of biological substances. However, NIR imaging still suffers from optical absorption and autofluorescence, thus there is still a need to find alternative agents for imaging.

### SUMMARY

Embodiments of the present disclosure provide for compositions including organic, water-soluble NIR-II fluorescent agent that emit radiation at about 1.0 to 1.7 μm, methods of making the composition, methods of imaging a disease and related biological events, methods of imaging, monitoring and/or assessing a disease and related biological events, and the like.

One exemplary embodiment of a composition, among others, includes: a water-soluble NIR-II fluorescent agent, optionally including one or more hydrophilic polymers, and a NIR-II emitting fluorophore, wherein the NIR-II emitting fluorophore emits light in the range of 1.0 to 1.7 μm under light excitation.

One exemplary embodiment of a composition, among others, includes: a water-soluble NIR-II fluorescent agent made of conjugated polymers including a D-A copolymer functionalized with a surfactant, wherein the D-A copolymer emits fluorescence at 1.0 to 1.7 μm.

One exemplary embodiment of a method of imaging, among others, includes: exposing a subject or sample to an excitation light source, wherein the subject was administered or sample was exposed to a composition, wherein the composition includes a water-soluble NIR-II fluorescent agent, wherein the water-soluble NIR-II fluorescent agent has an affinity for a target; and detecting the composition in the subject or sample using an imaging or detection device, wherein the location of the composition correlates to the location of the target.

One exemplary embodiment of a method of imaging blood vessels, among others, includes: exposing a subject to an excitation light source, wherein the subject was administered a composition that includes a water-soluble NIR-II fluorescent agent; and detecting the composition in the blood vessels using an imaging or detection device.

One exemplary embodiment of a method of in vivo fluorescence imaging of blood flow, among others, includes: exposing a subject to an excitation light source, wherein the subject is administered a composition that includes a water-soluble NIR-II fluorescent agent; and imaging the composition in the blood vessels to measure the blood flow in a blood vessel using an imaging or detection device.

Other composition, methods, systems, features, and advantages of the present disclosure will be or become apparent to one with skill in the art upon examination of the following detailed description. It is intended that all such additional devices, systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure.

FIG. 1.1 illustrates the synthesis of biocompatible IR-PEG nanoparticles with fluorescence emission>1000 nm. A) A reaction scheme illustrating the synthetic procedure of IR-PEG nanoparticles with IR-1061 embedded in polyacrylic acid matrix coated by PEGylated phospholipids (DSPE-mPEG). B) An AFM image of the synthesized nanoparticles deposited on a silicon substrate. C) Topographic height histogram showing the size distribution of ˜100 randomly selected nanoparticles.

FIG. 1.2 illustrates the spectral properties of free IR-1061 molecules in DMSO and IR-PEG nanoparticles in water. (A) UV-Vis-NIR absorption spectra (600-1500 nm) and (B) NIR-II fluorescence spectra (excited by an 808-nm laser) of IR-1061 in DMSO (top row) and IR-PEG nanoparticles in water (bottom row).

FIG. 1.3 illustrates in vivo NIR-II imaging of a nude mouse with intravenously injected IR-PEG nanoparticles. NIR-II fluorescence images of the entire body of the mouse recorded at various times (A-C) (as indicated) immediately after injection of the IR-PEG nanoparticles into the mouse tail vein, along with a PCA-overlaid image (D) based on the first 200 consecutive images from video-rate NIR-II imaging.

FIG. 1.4 illustrates in vivo NIR imaging of the hindlimb and belly of nude mice with fluorophores circulating in the blood streams post intravenous injection. (A) A mouse hindlimb image taken in the ˜800 nm NIR-I window with IRDye800 injection (see Supplementary Information). (B) and (C) are images recorded in the 1.1-1.7 μm and 1.3-1.7 μm NIR-II regions respectively post injection of IR-PEG nanoparticles under an 808 nm laser excitation. (D) An abdominal NIR-II fluorescence image taken in the 1.3-1.7 μm NIR-II region under excitation of a 980-nm laser after injection of IR-PEG nanoparticles.

FIG. 1.5 illustrates NIR-II fluorescence spectra excited by a 980-nm laser of IR-1061 in DMSO (top row) and IR-PEG nanoparticles in water (bottom row). Here we used a 980-nm laser to excite both samples and collected emission in the 1.1-1.5 μm region. IR-1061 in DMSO emitted fluorescence centered at 1135 nm, and IR-PEG nanoparticles in water revealed an emission peak at 1110 nm. Given the emission spectra of IR-PEG nanoparticles excited by 808-nm laser (see FIG. 1.2B), a red shift of the emission peak was observed for the IR-PEG nanoparticles in water when 980-nm laser was applied, owing to the choice of longer wavelength excitation, while the acquired fluorescence intensities by both lasers were comparable.

FIG. 1.6 illustrates fluorescence quantum yield of the IR-PEG nanoparticles. (A) UV-Vis-NIR absorption spectra of different concentrations of IR-26 in DCE. 1 mg/mL IR-26 in 1,2-dichloroethane (DCE) was diluted to a series of solutions in DCE with absorbance values at 808 nm of ˜0.10 (Pink), ˜0.08 (Green), ˜0.06 (Blue), ˜0.04 (Red) and ˜0.02 (Black), respectively. A total of five solutions with linearly spaced concentrations were loaded into a 10-mm fluorescence cuvette one at a time, and the exact absorbance for each solution was listed in the inset table. (B) NIR emission spectra of different IR-26 DCE solutions. For IR-26 DCE solutions with OD value=˜0.10, ˜0.08, ˜0.06, ˜0.04 and ˜0.02, their emission spectra were taken when the 808-nm laser was used as the excitation and a 900-nm long-pass filter was used as the emission filter to acquire the emission spectrum in the 900-1500 nm region, shown in pink, green, blue, red and black, respectively. Area under curve (AUC) in the emission spectra for each solution was then calculated and listed in the inset table. (C) For all IR-26 DCE solutions, their absorbance values were then plotted versus AUC, and fitted into a linear function, where slope of the fitted line was read as indicated. (D-F) The same absorption (D) and emission (E) measurements were performed for IR-PEG nanoparticles in aqueous solutions. (F) AUC in the emission spectrum of each solution of IR-PEG nanoparticles was then plotted against their absorbance at 808 nm and fitted into a linear function, as slope was indicated.

FIG. 1.7 illustrates the photostability of IR-PEG nanoparticles in aqueous solution. Photobleaching curves of 10 μm IR-1061 in DMSO (black line) and IR-PEG particles in water (red line) when exposed to continuous illumination at 808 nm for 1 h. Photoluminescence intensity was normalized by dividing the fluorescence intensity of each time point by the fluorescence intensity at t=0.

FIG. 2.1 illustrates the characterization of pDA-PEG nanoparticle. (A) A schematic of the pDA-PEG nanoparticle showing a hydrophobic polymer core and hydrophilic PEG shell. (B) A typical AFM image of pDA-PEG nanoparticles deposited on a silicon substrate, showing uniform size distribution of the polymeric nanoparticles. (C) Absorption and emission spectra of pDA-PEG, featuring a large Stokes shift of ˜400 nm with the NIR-II fluorescence emission at 1047 nm. (D) An NIR-II fluorescence image of an aqueous solution of pDA-PEG taken in the range of 1.0-1.7 μm NIR-II window under an excitation of 808 nm. (E) Fluorescence stability of pDA-PEG in different media including water, PBS and serum, suggesting negligible fluorescence quenching from water to biologically relevant media. (F) Photostability curves of pDA-PEG in water, PBS and serum under continuous illumination of an 808-nm laser. pDA-PEG in serum exhibits the lowest degree of photobleaching (<10%) among the three.

FIG. 2.2 illustrates the molecular Cell Imaging with pDA-PEG-Erbitux. (A) A schematic showing the structure of pDA-PEG-Erbitux bioconjugate, where the anti-EGFR antibody (Erbitux) selectively targets EGFR on the cell membrane of an MDA-MB-468 cell. (B & C) White-light (B) and NIR-II (C) fluorescence images of EGFR positive MDA-MB-468 cells incubated with the pDA-PEG-Erbitux bioconjugate, showing positive staining of cells. (D & E) White-light (D) and NIR-II (E) fluorescence images of EGFR negative U87-MG cells incubated with the pDA-PEG-Erbitux bioconjugate, without obvious staining of the cells. The scale bar in e indicates 40 μm, which applies to all images shown in B-E. (F) Average NIR-II fluorescence of EGFR positive MDA-MB-468 cells and negative U87-MG cells, showing a positive/negative ratio of ˜5.8. The error bars in F were obtained by taking the standard deviation of average fluorescence intensity from 20 cells in each NIR-II fluorescence image.

FIG. 2.3 illustrates the ultrafast video-rate imaging and tracking of blood flow in femoral artery of mouse. (A) A time course of NIR-II fluorescence images of a mouse hindlimb immediately following intravenous injection of pDA-PEG, showing the blood flow front moving inside the femoral artery. The frame rate of imaging is 25.6 fps with an exposure time of 20 ms and an instrument overhead time of 19 ms. (B) A plot of the distance travelled by the blood flow front as a function of time. The linear fit reveals an average blood velocity of 4.36 cm/s in the femoral artery. (C) A plot of instantaneous velocity (derived by dividing flow front traveled distance between two consecutive frames by the time interval of 39 ms) as a function of time, revealing periodic changes of instantaneous velocity corresponding to cardiac cycles. (D) An NIR-II fluorescence image of the same mouse hindlimb at 39 s after full perfusion of pDA-PEG containing blood into the hindlimb, upon which the fluorescence intensity in the hindlimb became unchanging.

FIG. 2.4 illustrates resolving blood flow pattern within a single cardiac cycle with ultrafast video-rate NIR-II imaging in vivo. (A & B) An NIR-II fluorescence image (a) of the mouse femoral artery, where the fluorescence intensity inside the region of interest (ROI) red box is integrated and plotted as a function of time in (B), showing an increasing profile with humps corresponding to ventricular ejections of cardiac cycles. (C) NIR-II fluorescence intensity plotted as a function of time, after a linear increasing baseline is subtracted from the plot shown in b, featuring 5 cardiac cycles in the plot. (D-J) Time course NIR-II fluorescence images of the red box area shown in a, after subtraction of a time-dependent linearly-increasing background given by the baseline shown in b. Note that these seven images correspond to a complete cardiac cycle from 312 ms to 546 ms. (K) Time point of NIR-II fluorescence spikes corresponding to ventricular ejections shown in c, plotted over several heart pulses (black squares). The data is fitted to linear function with its slope of 206.7 ms corresponding to the period of each cardiac cycle.

FIG. 2.5 illustrates a scheme for the Synthesis of pDA Polymer. This scheme shows the chemical structures of the two monomers M1 and M2, along with the structure of the pDA polymer, poly(benzo[1,2-b:3,4-b′]difuran-alt-fluorothieno-[3,4-b]thiophene).

FIG. 2.6 illustrates the ¹H NMR spectrum of the monomer M1. The peaks in the NMR spectrum are assigned as follows: 2.95 (t, 2H), 1.76 (m, 2H), 1.39-1.30 (m, 10H), 0.91 (t, 3H).

FIG. 2.7 illustrates the ¹⁹F NMR spectrum of the monomer M1. The peak in the ¹⁹F NMR spectrum is assigned as follows: −129 ppm (s, Ar—F)

FIG. 2.8 illustrates the ¹H NMR spectrum of the monomer M2. The peaks in the NMR spectrum are assigned as follows: 7.06 (s, 2H), 4.35 (d, 4H), 1.78 (m, 2H), 1.37-1.70 (m, 16H), 0.96-1.03 (m, 12H), 0.44 (s, 18H).

FIG. 2.9 illustrates the ¹H NMR spectrum of the pDA polymer. The peaks in the NMR spectrum are assigned as follows: 6.80 (br, 2H), 4.31 (br, 4H), 3.06 (br, 2H), 2.01-1.21 (br, 30H), 0.81-1.21 (br, 15H). The inset shows a zoomed-in view of the spectrum in the 2.5-7.3 ppm region.

FIG. 2.10 illustrates the gel permeation chromatography (GPC) spectrum of the pDA polymer. Molecular weight and polydispersity of polymer are listed as follows: Mₙ (number-average molecular weight): 16,192; M_{w} (weight-average molecular weight): 30,991; Mₚ (peak molecular weight): 24,941; PDI (polydispersity index):1.91.

FIG. 2.11 illustrates the size analysis of pDA-PEG. (A) A bar chart showing the particle size histogram of dried pDA-5k-PEG nanoparticles from the height measurements of the AFM image shown in (red bars), and the size distribution of the same pDA-5k-PEG sample in a 1×PBS solution based on DLS measurement (blue bars). (B) The size breakdown of the pDA-5k-PEG complex showing the polymer core of ˜3 nm (measured by AFM for the collapsed nanoparticle) and the overall size of ˜17 nm when the PEG chains were hydrated and extended (measured by DLS in an aqueous solution).

FIG. 2.12 illustrates quantum yield measurements. (A) UV-Vis-NIR absorption spectra of a series of the IR-26 reference solutions in DCE with increasing concentrations. (B) NIR-II emission spectra of the IR-26 reference solutions shown in A under an excitation of 808 nm. (C) Integrated NIR-II fluorescence intensity plotted as a function of absorbance at 808 nm for IR-26 reference solutions based on the measurements in A and B. The data was fitted into a linear function with a slope of 1.488×106. (D) UV-Vis-NIR absorption spectra of a series of the pDA-PEG polymer solutions in water with increasing concentrations. (E) NIR-II emission spectra of the pDA-PEG polymer solutions shown in D under an excitation of 808 nm. (F) Integrated NIR-II fluorescence intensity plotted as a function of absorbance at 808 nm for pDA-PEG polymer solutions based on the measurements in D and E. The data was fitted into a linear function with a slope of 5.870×106, giving a measured quantum yield of 1.7±0.11%. (G) UV-Vis-NIR absorption spectra of a series of SWNT solutions in water with increasing concentrations. (H) NIR-II emission spectra of the SWNT solutions shown in G under an excitation of 808 nm. (I) Integrated NIR-II fluorescence intensity plotted as a function of absorbance at 808 nm for SWNT solutions based on the measurements in G and H. The data was fitted into a linear function with a slope of 1.350×106, giving a measured quantum yield of 0.4±0.01%.

FIG. 2.13 illustrates the photostability study of the pDA-PEG polymer in different media. The top row shows the NIR-II fluorescence images of pDA-PEG polymer in water, PBS and serum before continuous 808-nm excitation, at the same concentration of 7.5 μg/mL. The bottom row shows the NIR-II fluorescence images of the same samples after continuous 808-nm excitation for 1 h.

FIG. 2.14 illustrates the origin of ‘dot’ features along the femoral artery in the baseline-subtracted NIR-II fluorescence images shown in FIG. 2.4. (A) A NIR-II fluorescence image at 390 ms p.i., corresponding to the red box area shown in FIG. 2.4A, after subtraction of the baseline shown in FIG. 2.4B. (B) The same basedline-subtracted NIR-II image as in A, with all ‘dot’ features labeled as A, C, E, G, I and K, and the locations between two neighboring dots labeled as B, D, F, H and J. (C) NIR-II fluorescence intensity and vessel diameter measured at all points labeled from A to K in B, showing in-phase changes between the two measurements at these points. A positive correlation between the local blood volume and the NIR-II fluorescence intensity has been found, suggesting a larger lumen (i.e., a larger diameter) of the vessel could hold more blood with more NIR-II fluorescent agents, and thus appeared brighter than neighboring vessel segments, resulting in the ‘dot’ features after subtracting a constant baseline from all pixels in the NIR-II fluorescence image.

FIG. 2.15 illustrates pDA Polymers with Tunable Absorption and Emission Properties. The chemical structures of four different pDA molecules are shown in A-D, along with their absorption and emission spectra (E & F) revealing the emission wavelengths tuned in the range of 1050-1350 nm.

FIG. 2.16 illustrates the blood flow tracking inside a capillary vessel. (A) Time course NIR-II fluorescence images of a capillary vessel after injection of pDA-PEG nanoparticles under an excitation of 808 nm. A bolus of injected pDA-PEG travelling upwards along a 6-μm wide capillary vessel (outlined in red dashed lines) can be visualized in the images. The scale bar indicates 20 μm. (B) A plot of the distance travelled by the bolus of injected pDA-PEG as a function of time. The linear fit reveals an average blood velocity of 55.2 μm/s in this particular capillary vessel, ˜800× slower than in the femoral artery and also much less affected by the cardiac output cycles.

FIG. 2.17 illustrates the imaging regional blood redistribution with pDA-PEG. (A) A white light image showing the mouse with heat-induced inflammation in the right hindlimb (left to the viewer). This image was taken at ˜5 h after the inflammation was induced. (B-H) Time course NIR-II fluorescence images of the mouse at different times after the inflammation was induced. The scale bar in B indicates 10 mm and applies to all NIR-II fluorescence images. (I) Average NIR-II fluorescence intensity plotted as a function of time after induced inflammation in the control and the inflamed hindlimbs. The error bars in I were obtained by taking the standard deviation of the NIR-II fluorescence intensity of all pixels in each hindlimb at every time point after induced inflammation. (J) The ratio of the NIR-II fluorescence intensity in the inflamed hindlimb over that in the control hindlimb, plotted as a function of time after the induced inflammation. The error bars in J were obtained by propagating the errors associated with the corresponding control and inflamed hindlimbs shown in I.

FIG. 2.18 illustrates the size tunability of the pDA-PEG nanoparticles. This plot shows the size distribution of the pDA-PEG nanoparticles measured by DLS and plotted as a function of initial pDA concentration in the THF solution during the synthesis of pDA-PEG nanoparticles and the molecular weight of the surfactant DSPE-mPEG. Note that the measured hydrodynamic diameters of pDA-2k-PEG in the range of 2-6 nm (the bottom left data point) may contain some ‘empty’ surfactant micelles without any pDA molecules loaded inside (2-4 nm in diameter). The error bars reflect the standard deviation of the measured hydrodynamic diameter distribution of each corresponding sample.

FIG. 3.1 illustrates the imaging in various NIR sub-regions. (A) A fluorescence emission spectrum of SWNT-IRDye800 conjugate in the range of 850-1650 nm under the excitation of an 808-nm laser. The emission spectra of IRDye800 and SWNT are plotted under different y axis scales to accommodate both into the same graph, due to the much higher fluorescence intensity of IRDye800 than SWNTs. (B) NIR fluorescence images of a capillary tube filled with SWNT-IRDye800 solution immersed at depths of 1 mm (top) and 10 mm (bottom) in 1% Intralipid recorded in NIR-I, NIR-II and NIR-IIa regions respectively. (c) The extinction spectrum (black curve) and scattering spectrum (red curve, measured by subtracting water and Intralipid absorptions from the extinction spectrum, see FIG. 3.6) of 1% Intralipid in water with path length of 1 mm measured by UV-Vis-NIR spectrometer, along with reduced scattering coefficient profile (blue) of 1% Intralipid derived from literature.

FIG. 3.2 illustrates the in vivo mouse brain imaging with SWNT-IRDye800 in different NIR sub-regions. (A) A C57Bl/6 mouse head with hair removed. (B) A schematic of the NIR-IIa fluorescence imaging rig. (C-E) Fluorescence images of the same mouse head in the NIR-I, NIR-II and NIR-IIa regions. (F) Extinction spectra of scalp (red), skull (blue) and brain tissue (pink) along with the water absorption spectrum (black).

FIG. 3.3 illustrates the non-invasive, high-resolution NIR-IIa fluorescence imaging of mouse brain vasculature. (A) A photo showing the stereotactic microscopic imaging setup, where a red laser is used for alignment and shows the beam location. (B) A schematic showing the penetration of NIR-IIa fluorescence through brain tissue, skull and the scalp. (C) A photoluminescence versus excitation (PLE) spectrum of LS nanotubes in an aqueous solution. The 1.3-1.4 μm NIR-IIa region is shaded red. (D) A low-magnification cerebral vascular image taken with a field of view of 25 mm×20 mm. (E) A cerebral vascular image of the same mouse head zoomed into the left cerebral hemisphere, with a field of view of 8 mm×6.4 mm. (F) A cerebral vascular image of the same mouse head taken using a microscope objective, with a field of view of 1.7 mm×1.4 mm. The depth of these in-focus vascular features was determined as 2.6 mm. (G) A zoomed-in image of a sub-region in F taken by a higher magnification objective, with a field of view of 0.80 mm×0.64 mm. The inset shows the cross-sectional intensity profile (black) and Gaussian fit (red) along the yellow-dashed bar. (H-K) Two other high resolution cerebral vascular images with a field of view of 0.80 mm×0.64 mm taken on another mouse (H&J), and their cross-sectional fluorescence intensity profiles (black) and Gaussian fit (red) along the yellow-dashed bars (I&K).

FIG. 3.4 illustrates the dynamic NIR-IIa fluorescence imaging of mouse cerebral vasculature. (A-C) Time course NIR-IIa images of a control, healthy mouse (Mouse C1). (D-F) PCA overlaid images showing arterial (red) and venous (blue) vessels of Mouse C1. (G-I) Time course NIR-IIa images of a mouse with MCAO (Mouse M1). (J-L) PCA overlaid images showing arterial (red) and venous (blue) vessels of Mouse M1. (M-N) Normalized NIR-IIa signal in the left (red) and right (black) cerebral hemispheres of Mouse C1 (M) and M1 (N) versus time. (O) Average blood perfusion of the left cerebral hemisphere of control group (n=3), MCAO group (n=4) and cerebral hypoperfusion group (n=4), measured by NIR-II method (red) and laser Doppler blood spectroscopy (blue). Errors bars reflect the standard deviation of each group.

FIG. 3.5 illustrates NIR fluorescence images of a capillary tube filled with SWNT-IRDye800 solution immersed at depths of 1 mm (A, E, I), 4 mm (B, F, J), 7 mm (C, G, K) and 10 mm (D, H, I) in 1% Intralipid recorded in NIR-I (A-D), NIR-II (E-H) and NIR-IIa (I-L) regions respectively.

FIG. 3.6 illustrates the determination of the scattering spectrum of 1% Intralipid in water. The scattering spectrum (red curve) was obtained by subtracting the absorption of water (green curve) and absorption of 1% Intralipid in an organic solvent (blue curve; acetone forms a non-scattering solution for Intralipid) from the as-measured extinction spectrum of 1% Intralipid aqueous solution (black curve).

FIG. 3.7 illustrates a schematic of the NIR-IIa fluorescence imaging rig for non-invasive through-scalp and through-skull brain vascular imaging.

FIG. 3.8 illustrates the physical measurement of the total thickness of scalp skin, cranial bone and the meninges of a C57Bl/6 mouse. (a) A digital camera photo of a dissected mouse head. The scalp, skull and meninges over the right cerebral hemisphere were completed removed while those over the left hemisphere remained intact. Note that the mouse was perfused with 5% Evans blue before sacrifice to render the vessels blue in the brain. (b-d) A repeated measure of the total thickness of scalp skin, cranial bone and the meninges by digital calipers at three different locations. The average thickness was 1.37 mm, setting the lower bound of cerebral vessel depths in our imaging experiments. Therefore the cortical vessels located at the surface of the brain, including the inferior cerebral vein, the superior sagittal sinus and the transverse sinus, should have depths within the range of 1-2 mm underneath the scalp skin.

FIG. 3.9 illustrates the photoluminescence versus excitation (PLE) spectra of unseparated HiPCO SWNTs (top) and separated LS nanotubes (bottom) in aqueous solutions. The 1.3-1.4 μm NIR-IIa region is shaded red in both spectra to highlight the difference of fluorescence emission of the two samples in this region (enhanced emission in the region for separated LS nanotubes). Note that metallic nanotubes with no fluorescence were mostly removed in the separated LS sample.

FIG. 3.10 illustrates more microscopic images of brain vessels recorded in the NIR-IIa region at depths in the range of 1-3 mm underneath the surface of the scalp skin of several mice. Images A & B were taken under a 4× microscopic objective with a field of view of 1.7 mm×1.4 mm, while images C-F were taken under a 10× microscopic objective with a field of view of 800 μm×640 μm. Cross-sectional fluorescence intensity profiles (black) along the green-dashed bars in the images are shown under each corresponding image, and Gaussian fitted peaks to the profiles are shown as the red curves, featuring capillary vessel widths in the range of 6-16 μm.

FIG. 3.11 illustrates statistical analysis of capillary vessel widths in NIR-IIa fluorescence images. (A-F) Microscopic cerebral vascular images with the analyzed capillary vessels intersected with green dashed bars. (G) A histogram showing the distribution of measured capillary vessel widths in the range of 5-15 μm, with a mean vessel width of 9.4 μm and a standard deviation of 2.5 μm.

FIG. 3.12 illustrates NIR-IIa fluorescence images taken at different time points post injection (p.i.), between which we returned the mouse to its cage for it to recover from anesthesia. These time-course images show a time window of ˜3 h p.i. for static brain vessel imaging. Note that in the last two images a tape was applied on part of the scalp skin to cover a wound possibly caused by the bite from another mouse in the same cage.

FIG. 3.13 illustrates dynamic NIR-IIa fluorescence imaging of mouse cerebral vasculature on a group of control mice (Mouse C2 & C3) at a frame rate of 5.3 frames·s-1. (A-C) Time course NIR-IIa fluorescence images showing blood flow in the cerebral vasculature of Mouse C2. (D) PCA overlaid image based on the first 100 frames (18.75 s post injection) of Mouse C2. (E-G) Time course NIR-IIa fluorescence images showing blood flow in the cerebral vasculature of Mouse C3. (H) PCA overlaid image based on the first 100 frames (18.75 s post injection) of Mouse C3. (I) Normalized NIR-IIa signal in the left (red) and right (black) cerebral hemispheres of Mouse C2 versus time. (J) Normalized NIR-IIa signal in the left (red) and right (black) cerebral hemispheres of Mouse C3 versus time.

FIG. 3.14 illustrates dynamic NIR-IIa fluorescence imaging of mouse cerebral vasculature on a group of MCAO mice (Mouse M2-4) at a frame rate of 5.3 frames·s-1. (A-C) Time course NIR-IIa fluorescence images showing blood flow in the cerebral vasculature of Mouse M2. (D) PCA overlaid image based on the first 100 frames (18.75 s post injection) of Mouse M2. (E-G) Time course NIR-IIa fluorescence images showing blood flow in the cerebral vasculature of Mouse M3. (H) PCA overlaid image based on the first 100 frames (18.75 s post injection) of Mouse M3. (I-K) Time course NIR-IIa fluorescence images showing blood flow in the cerebral vasculature of Mouse M4. (L) PCA overlaid image based on the first 100 frames (18.75 s post injection) of Mouse M4. (M) Normalized NIR-IIa signal in the left (red) and right (black) cerebral hemispheres of Mouse M2 versus time. (N) Normalized NIR-IIa signal in the left (red) and right (black) cerebral hemispheres of Mouse M3 versus time. (0) Normalized NIR-IIa signal in the left (red) and right (black) cerebral hemispheres of Mouse M4 versus time.

FIG. 3.15 illustrates dynamic NIR-IIa fluorescence imaging of mouse cerebral vasculature on a group of mice with cerebral hypoperfusion (Mouse H1-4) at a frame rate of 5.3 frames·s-1. (A-C) Time course NIR-IIa fluorescence images showing blood flow in the cerebral vasculature of Mouse H1. (D) PCA overlaid image based on the first 100 frames (18.75 s post injection) of Mouse H1. (E-G) Time course NIR-IIa fluorescence images showing blood flow in the cerebral vasculature of Mouse H2. (H) PCA overlaid image based on the first 100 frames (18.75 s post injection) of Mouse H2. (I-K) Time course NIR-IIa fluorescence images showing blood flow in the cerebral vasculature of Mouse H3. (L) PCA overlaid image based on the first 100 frames (18.75 s post injection) of Mouse H3. (M-O) Time course NIR-IIa fluorescence images showing blood flow in the cerebral vasculature of Mouse H4. (P) PCA overlaid image based on the first 100 frames (18.75 s post injection) of Mouse H4. (Q) Normalized NIR-IIa signal in the left (red) and right (black) cerebral hemispheres of Mouse H1 versus time. (R) Normalized NIR-IIa signal in the left (red) and right (black) cerebral hemispheres of Mouse H2 versus time. (S) Normalized NIR-IIa signal in the left (red) and right (black) cerebral hemispheres of Mouse H3 versus time. (T) Normalized NIR-IIa signal in the left (red) and right (black) cerebral hemispheres of Mouse H4 versus time.

### DETALED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, molecular imaging, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

In describing and claiming the disclosed subject matter, the following terminology will be used in accordance with the definitions set forth below.

By “administration” or “administering” is meant introducing water-soluble NIR-II fluorescent agent of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used. Also, these terms can mean that the water-soluble NIR-II fluorescent agent is introduced to a sample using an appropriate technique for the sample.

In accordance with the present disclosure, “a detectably effective amount” of the water-soluble NIR-II fluorescent agent of the present disclosure is defined as an amount sufficient to yield an acceptable image using equipment that is available for clinical use. A detectably effective amount of the water-soluble NIR-II fluorescent agent of the present disclosure may be administered in more than one injection. The detectably effective amount of the water-soluble NIR-II fluorescent agent of the present disclosure can vary according to factors such as the degree of susceptibility of the individual, the age, sex, and weight of the individual, idiosyncratic responses of the individual, and the like. Detectably effective amounts of the water-soluble NIR-II fluorescent agent of the present disclosure can also vary according to instrument and film-related factors. Optimization of such factors is well within the level of skill in the art.

As used herein, the term “patient” or “subject” includes humans, poultry, and mammals (e.g., cats, dogs, horses, etc.) and their cells and tissues as well as non-mammalian organisms or animals. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a subject. The term “living subject” refers to a subject noted above that is alive and is not dead. The term “living subject” refers to the entire subject and not just a part excised (e.g., a liver or other organ) from the living subject.

The term “sample” can refer to a tissue sample, cell sample, a fluid sample, and the like. The sample may be taken from a subject. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. In the present disclosure, the source of the sample is not critical.

The term “detectable” refers to the ability to detect a signal over the background signal.

The term “near-infrared-II (or NIR-II)” (or sometimes referred to as the “second near-infrared window”) refers to the spectral range of 1000-1700 nm in wavelength and is different from the traditional 700-800 nm NIR region. The term “NIR-IIa” refers to the 1300-1400 nm spectral region, which is located right before a substantial water absorption peak. The term “NIR-IIb” refers to the 1500-1700 nm spectral region, which is right after a substantial water absorption peak and right before another major water absorption peak.

The term “detectable signal” is a signal derived from non-invasive imaging techniques such as, but not limited to, NIR imaging. The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between the detectable signal and the background. Standards and/or calibration curves can be used to determine the relative intensity of the detectable signal and/or the background.

### General Discussion

Embodiments of the present disclosure provide for compositions including organic, water-soluble NIR-II fluorescent agent that emit radiation at about 1.0 to 1.7 μm, methods of making the composition, methods of imaging a disease and related biological events, methods of imaging, monitoring and/or assessing a disease and related biological events, and the like. In particular, the water-soluble NIR-II fluorescent agent of the present disclosure can include an NIR-emitting agent(s) (also referred to as “NIR-II emitting agent”) or the donor-acceptor (D-A) copolymer emits light, where each emits light at about 1.0 to 1.7 μm (e.g., 1.3-1.4 μm or 1.5 to 1.7 μm). Compositions including water-soluble NIR-II fluorescent agents can be used in in vivo and in vitro imaging of a target such as a disease (e.g., cancer or cardiovascular diseases). Embodiments of these compositions are advantageous because they combine water solubility and low optical scattering, low absorption, and low autofluorescence by endogenous tissues, and consequently should be able to be imaged in deep tissue with high sensitivity, and high spatial and temporal resolution. In addition, the size and/or morphology of the water-soluble NIR-II fluorescent agents can be controlled so that they have a uniform distribution, the surface of the water soluble nanoparticles can be easily modified for specific targeting and/or rapid body clearance by either fecal or renal routes for particles with <5.5 nm hydrodynamic diameters, and the fluorescence quantum efficiency of the NIR-II emitting agent is preserved.

In an embodiment, the water-soluble NIR-II fluorescent agent can include a hydrophilic polymer and a NIR-II-emitting agent. In an embodiment, the NIR-II-emitting agent emits radiation at about 1.0 to 1.7 μm in NIR-II or at about 1.3 to 1.4 μm in NIR-IIa or about 1.5 to 1.7 μm in NIR-IIb. In an embodiment, the NIR-II emitting agent can include organic molecules, nanoparticles (e.g., nanotubes, quantum dots, etc.), and other molecules and particles that emit light at about 1.0 to 1.7 μm. In an embodiment, the polymer can be functionalized with a surfactant. An embodiment of the water-soluble NIR-II fluorescent agent can have a diameter (or longest dimension, length) of about 3 to 20 nm or about 3 to 10 nm and lengths up to hundreds of nanometers or about 1 micron. In an embodiment, the NIR-II fluorescent agent can have a hydrodynamic size smaller than about 5.5 nm or about 4 nm. In an embodiment, the NIR-II fluorescent agent has the characteristic of being able to be excreted through the renal system.

In an embodiment, the hydrophilic polymer can be selected from poly(acrylic acid) (PAA), poly(vinyl alcohol) (PVA), polyacrylamide, polyethylene glycol (PEG), and the like, as well as derivatives of each of these. In general, the polymer can have a molecular weight of about 2,000 to 16,000. In an embodiment, the polymer can be a PAA having a molecular weight of about 2000.

As mentioned above, the polymer can be functionalized with a surfactant so that the nanoparticle is water soluble. In an embodiment, the surfactant can include polyethylene glycol (PEG) based compound, a phospholipid-polyethylene glycol compound, n-MEG, poly(vinyl alcohol) (PVA), poly(acrylic acid) (PAA), poly(propylene furmarate-co-ethylene glycol) (P(PF-co-EG)), polyacrylamide, polypeptides, poly-N-substituted glycine oligomers (polypeptoids), and the like, as well as naturally derived polymers normally include hyaluronic acid (HA), alginate, chitosan, agarose, collagen, fibrin, gelatin, dextran, and any combination thereof, as well as derivatives of each of these. In an embodiment, the surfactant can be poly(ethylene glycol)-distearoylphosphatidylethanolamine (PEG-DSPE), 1,2-distearoyl-sn-glycero-3-phosphoethanolamine-N-[amino(polyethylene glycol) (PEG-DSPE-amine), C18-PEG, and a combination thereof.

In an embodiment, the PEG can be a linear PEG, a multi-arm PEG, a branched PEG, and combinations thereof. The molecular weight of the PEG can be about 2 kDa to 90 kDa. When used in reference to PEG moieties, the word “about” indicates an approximate average molecular weight and reflects the fact that there will normally be a certain molecular weight distribution in a given polymer preparation.

In an embodiment, the NIR-II-emitting agents can be encapsulated within and/or disposed on the surface (e.g., partially) by hydrophilic polymers to form water-soluble nanoparticle, but preferably, the NIR-II emitting agents are disposed within the nanoparticle. In an embodiment, the nanoparticle can include one or more types of NIR-II emitting agents. In an embodiment, the nanoparticle can include up to 10 to 100 NIR-II emitting agents.

In an embodiment, the NIR-II emitting agents within the particles or complexes can include, but are not limited to, small organic molecule based polymethine dyes (e.g., IR1061, IR 26, and IR 1051) (and derivatives thereof) that emit at about 1.0 to 1.7 μm, cyanine dyes (and derivatives thereof) that emit at about 1.0 to 1.7 μm. In an embodiment, the NIR-emitting agent can include the following that emit at about 1.0 to 1.7 μm: BODIPY® fluorophores (Molecular Probes) (e.g., 4,4-difluoro-4-bora-3a,4a-diaza-s-indacene (and derivatives thereof), which can be modified to alter the wavelength (BODIPY® substitutes for the fluorescein, rhodamine 6G, tetramethylrhodamine and Texas Red fluorophores are BODIPY® FL, BODIPY® R6G, BODIPY® TMR and BODIPY® TR, respectively)), 1H,5H,11H,15H-Xantheno[2,3,4-ij:5,6,7-i′j′]diquinolizin-18-ium, 9-[2(or 4)-(chlorosulfonyl)-4(or 2)-sulfophenyl]-2,3,6,7,12,13,16,17-octahydro-, inner salt (molecular formula: C₃₁H₂₉ClN₂O₆S₂) (and derivatives thereof) (Texas Red), and Xanthylium, 3,6-diamino-9-(2-(methoxycarbonyl) phenyl, chloride (C₂₁H₁₇ClN₂O₃) (and derivatives thereof) (NIR Rhodamine dye), where derivatives of each can be used to modify the wavelength, as long as it is within the noted range of the present disclosure. In particular, the NIR dye can include, but is not limited to, NIR775, BODIPY® dye series (e.g., BODIPY® FL-X, BODIPY® R6G-X, BODIPY® TMR-X, BODIPY® TR-X, BODIPY® 630/650-X, and BODIPY® 650/665-X (Molecular Probes, Inc. Eugene, Oreg., USA)), NIR Rhodamine dyes, NIR Alexa® dyes (e.g., Alexa® Fluor 350, Alexa® Fluor 405, Alexa® Fluor 430, Alexa® Fluor 488, Alexa® Fluor 500 (Molecular Probes, Inc. Eugene, Oreg., USA)), ADS dyes (e.g., ADS775®, ADS 780®, and the like), Texas Red, or cyanine dyes (e.g., Cy5.5 Cy3, Cy5), and Li-Cor IRDye™ products.

In an embodiment, the NIR-II emitting agents can include, but are not limited to, a nanotube that emits from about 1.0 to 1.7 μm such as a carbon nanotube. In an embodiment, carbon nanotubes are generally described as large elongated fullerenes of closed-cage carbon molecules typically arranged in hexagons and pentagons. In an embodiment, the carbon nanotubes can be single wall nanotubes (SWNT) or multi-walled nanotubes (MWNT). Embodiments of the MWNT can include 2 or more walls, 5 or more walls, 10 or more walls, 20 or more walls, or 40 or more walls. In an embodiment, the carbon nanotubes including SWNTs and MWNTs may have diameters from about 0.6 nanometers (nm) up to about 3 nm, about 5 nm, about 10 nm, about 30 nm, about 60 nm or about 100 nm. In an embodiment, the single-wall carbon nanotubes may have a length from about 50 nm up to about 1 micro-meter (μm), or greater. In an embodiment, the diameter of the single-wall carbon nanotube is about 0.7 to 5 nm and has a length of about 50 to 500 nm. In an embodiment, the SWNT emits fluorescence at a range of 1.3-1.4 μm NIR-IIa with diameters in the range of 1.0 nm to 1.2 nm and can be made by HIPCO method, arc-discharge method and other methods. In an embodiment, the SWNT emits fluorescence in at range of 1.5 to 1.7 μm NIR-IIb with diameters in the range of 1.2 nm to 1.5 nm and can be made by arc-discharge, laser ablation, chemical vapor deposition, method and other methods.

In an embodiment, the select nanotubes were separated from other nanotubes based on semiconductor/metallic nature and diameter so that the select nanotube emits light in the range of 1.0 to 1.7 μm under light excitation. In particular, large-diameter semiconducting (LS) nanotubes were separated from HIPCO raw materials, and these selected nanotubes emit light predominantly in the range of 1.3 to 1.4 μm NIR-IIa region under light excitation. In general, the raw nanotubes can be sonicated, separated using ultracentrifugation, and separated in a gel-filtration column, to collect the LS nanotubes. These nanotubes have a diameter in the range of 1.0 nm to 1.2 nm and can be separated from other diameter and metallic nanotubes. In an embodiment, even larger nanotubes having a diameter in the range of 1.2 nm to 1.5 nm can be separated from other diameter and metallic nanotubes, and the 1.2-1.5 nm SWNTs predominantly fluoresce in the NIR-IIb region, i.e., 1500-1700 nm. Additional details are provided in the Examples. In an embodiment, the NIR-II emitting agents can include both the NIR-II emitting dye and the nanotube described above.

In an embodiment, the water-soluble NIR-II fluorescent agent can be prepared using a one-pot chemical synthesis. In an embodiment, the NIR-II-emitting agent (such as the dyes) and the hydrophilic polymer (such as PAA) are mixed in a solvent (e.g., DMSO) and then mixed with a surfactant. The mixture is sonicated and the solvent and unreacted chemicals are removed using a technique such as filtration. The nanoparticles are suspended in water and washed. In an embodiment, the NIR-emitting agent is disposed in the center of the water-soluble NIR-II fluorescent agent, while the surfactant agent is directed towards the outside of the water-soluble NIR-II fluorescent agent, which enhances the water solubility of the water-soluble NIR-II fluorescent agent.

In an embodiment, the water-soluble NIR-II fluorescent agent can also include one or more types of agents bound (e.g., associated directly or indirectly) to the nanoparticle. The water-soluble NIR-II fluorescent agent can include one or more agents that can be used to enhance the interaction of the water-soluble NIR-II fluorescent agent with the subject or subject. In an embodiment, the agent (e.g., targeting agent) can have an affinity for a biological target such as, but not limited to, a compound, a polypeptide, a polynucleotide, an antibody, an antigen, a hapten, a cell type, a tissue type, and the like. In an embodiment, the agent may be an antigen specific for an antibody that corresponds to a certain disease or condition. In another embodiment, the agent may be a first protein specific for another protein. In another embodiment, the agent may be a polynucleotide sequence specific for a complementary polynucleotide sequence. In another embodiment, the agent can undergo a chemical, biological, and/or physical change, where the changed agent can have an affinity for a target.

In an embodiment, the targeting agent can include, but is not limited to, peptides, polypeptides (e.g., protein such as, but not limited to, an antibody (monoclonal or polyclonal)), nucleic acids (both monomeric and oligomeric), polysaccharides, sugars, fatty acids, steroids, purines, pyrimidines, drugs (e.g., small compound drugs), ligands, or combinations thereof. In an embodiment, the targeting agent can have an affinity for functional groups, compounds, cells, tissue, and the like, associated with a disease or condition. The targeting agent can have an affinity for one or more targets.

Thus, the agent can be selected so that the water-soluble NIR-II fluorescent agent can be used to image and/or diagnose the presence or absence of the compounds, polypeptides, polynucleotides, antibodies, antigens, haptens, cell types, tissue types, and the like, associated with a disease or condition, or related biological activities.

In addition, the agent can also include, but is not limited to, a drug, a therapeutic agent, a radiological agent, a small molecule drug, and combinations thereof, that can be used to treat the target molecule and/or the associated disease and condition of interest. The drug, therapeutic agent, and radiological agent can be selected based on the intended treatment as well as the condition and/or disease to be treated. In an embodiment, the water-soluble NIR-II fluorescent agent can include two or more agents used to treat a condition and/or disease.

In an embodiment, the water-soluble NIR-II fluorescent agent can include at least two different types of agents, one being a targeting agent that targets certain cells or compounds associated with a condition and/or disease, while the second agent is a drug used to treat the disease. In this manner, the water-soluble NIR-II fluorescent agent acts as a detection component, a delivery component to the cells of interest, and a delivery component for the treatment agent. The detection of the water-soluble NIR-II fluorescent agent can be used to ensure the delivery of the drug to its intended destination as well as the quantity of water-soluble NIR-II fluorescent agent delivered to the destination.

Additional details regarding the water-soluble NIR-II fluorescent agent and methods of making the water-soluble NIR-II fluorescent agent are described in Examples 1 and 3.

In another embodiment, a water-soluble NIR-II fluorescent agent can include conjugated polymers such as a donor-acceptor (D-A) copolymer functionalized with a surfactant, where the D-A copolymer emits radiation at 1.0 to 1.7 μm. In an embodiment, the surfactant can include those that are described in reference to the other embodiments described herein. In addition, the water-soluble NIR-II fluorescent agent can include agents (e.g., targeting agent) such as include those that are described in reference to the other embodiments described herein. An embodiment of a method of making the water-soluble NIR-II fluorescent agent is described in Example 2.

In an embodiment, the selection of the donor monomer and the acceptor monomer to form D-A copolymer can be made so that the D-A copolymer has a tunable bandgap energy such that the fluorescence emission falls in the range of 1.0 to 1.7 μm. In an embodiment, the D-A copolymer can include a donor monomer such as benzo[1,2-b:3,4-b′]difuran, or its derivatives with electron-donating substituents. In an embodiment, the D-A copolymer can include an acceptor monomer such as thieno-[3,4-b]thiophene, or its derivative with electron-withdrawing substituents. In an embodiment, the D-A copolymer can be poly {4,8-bis(2-ethylhexyloxy) benzo[1,2-b; 3,4-′b]difuran-alt-3-fluorothieno[3,4-b]thiophen-2-yl)nonan-1-one} or poly[(4,4′-bis(2-ethylhexyl)dithieno[3,2-b:″,3′-d]silole)-2,6-diyl-alt-3-fluorothieno[3,4-b]thiophen-2-yl)nonan-1-one].

Additional details regarding the water-soluble NIR-II fluorescent agent and methods of making the water-soluble NIR-II fluorescent agent are described in Example 2.

### Methods of Use

Embodiments of this disclosure include, but are not limited to: methods of imaging a sample or a subject using the water-soluble NIR-II fluorescent agent, methods of imaging a disease (e.g., presence or progression) or related biological events, methods of monitoring or accessing a disease treatment, and the like. In particular, embodiments of the present disclosure can be used to image, detect, study, monitor, evaluate, assess, and/or screen, a condition, a disease, a disease treatment, in vivo or in vitro using the water-soluble NIR-II fluorescent agent. When reference is made to “water-soluble NIR-II fluorescent agent”, any one of the water-soluble NIR-II fluorescent agents described herein can be used to accomplish the noted method. In addition, the water-soluble NIR-II fluorescent agent can include one or more agents, specifically targeting agents that target a specific disease or related biological event.

Embodiments of the present disclosure include methods for imaging a sample (e.g., tissue or cell(s)) or a subject (e.g., animal or human) that includes contacting a sample with or administering to a subject a water-soluble NIR-II fluorescent agent or a composition including a water-soluble NIR-II fluorescent agent. In an embodiment, the water-soluble NIR-II fluorescent agent can be excited with a laser or other light source with wavelength of about 500-1500 nm. Subsequently, the water-soluble NIR-II fluorescent agent can be imaged under optical excitation by a laser or other light sources using NIR imaging systems such as Princeton 2D InGaAs array or Hamamatsu 2D InGaAs image sensor, and the like. The imaging can be performed in vivo and/or in vitro. In an embodiment, the resolution obtained is less than 100 μm or about 10 μm. In an embodiment, an image can be obtained at about 1 to 3 mm into the sample or subject. In a particular embodiment, the water-soluble NIR-II fluorescent agent can be used in imaging a target disease, such as cancer, and determine its presence or progression/regression. For example, the water-soluble NIR-II fluorescent agent is provided or administered to a subject in an amount effective to result in uptake of the water-soluble NIR-II fluorescent agent. The subject is then introduced to an appropriate imaging system (e.g., NIR imaging system) for a certain amount of time (e.g., this depends on water-soluble NIR-II fluorescent agent being used, but it can be up to 3 min to 12 hours). The water-soluble NIR-II fluorescent agent is activated or excited (e.g., light source with wavelength of about 500-1500 nm) and emits radiation. The detected signal from the water-soluble NIR-II fluorescent agent in the range of 1 to 1.7 μm (e.g., 1.3-1.4 μm or 1.5 to 1.7 μm) can be correlated to the amount of water-soluble NIR-II fluorescent agent in one or more areas corresponding the target, which correlates with the presence of the target in that location(s) of the subject. In an embodiment, the steps of this method can be repeated at determined intervals (e.g., daily, weekly, monthly, intervals in between these, and the like) so the presence/absence/progress of the disease can be monitored as a function of time and/or treatment.

Embodiments of the present disclosure include methods for imaging blood vessels in a sample (e.g., tissue or cell(s)) or a subject (e.g., animal or human). In an embodiment, the subject can be intravenously administered a water-soluble NIR-II fluorescent agent or a composition including a water-soluble NIR-II fluorescent agent. In an embodiment, the water-soluble NIR-II fluorescent agent can be excited with a laser or other light source. Subsequently, the water-soluble NIR-II fluorescent agent can be imaged under optical excitation by a laser or other light sources using NIR imaging systems, as described herein, to image the blood vessel. In an embodiment, the resolution obtained can be less than 100 μm or 10 μm or even smaller (e.g., about 1 or 5 μm). In an embodiment, an image can be obtained at a depth of about 1 to 3 mm or even deeper (e.g., 5 mm or 10 mm) into the sample or subject.

Embodiments of the present disclosure include methods for imaging blood flow in a blood vessel in a subject (e.g., animal or human). In an embodiment, the subject can be intravenously administered a water-soluble NIR-II fluorescent agent or a composition including a water-soluble NIR-II fluorescent agent. In an embodiment, the water-soluble NIR-II fluorescent agent can be excited as described above. Subsequently, the water-soluble NIR-II fluorescent agent can be imaged under optical excitation by a laser or other light sources using NIR imaging system, such as that described herein, show the blood flow in the blood vessel. In an embodiment, the resolution obtained can be less than 100 μm or 10 μm or even smaller. In an embodiment, an image can be obtained at a depth of about 1 to 3 mm or deeper into the sample or subject. In particular, embodiments of the present disclosure enable dynamically imaging and tracking in real-time blood flow in a subject. In an embodiment, video-rate imaging of the subject can be performed with an ultrafast frame rate of about 25.6 frames per second (fps). Such a high imaging speed enables clear observation of the blood flow front (See Examples). Thus, in vivo blood flow velocity measurements can be obtained using fluorescence imaging in real-space and -time

It should be noted that the amount effective to result in uptake of the water-soluble NIR-II fluorescent agent into the subject (brain) may depend upon a variety of factors, including for example, the age, body weight, general health, sex, and diet of the subject; the time of administration; the route of administration; the rate of excretion of the specific water-soluble NIR-II fluorescent agent employed; the duration of the treatment; the existence of other drugs used in combination or coincidental with the specific composition employed; and like factors well known in the medical arts.

### Kits

The present disclosure also provides packaged compositions or pharmaceutical compositions comprising a pharmaceutically acceptable carrier and a water-soluble NIR-II fluorescent agent of the disclosure. In certain embodiments, the packaged compositions or pharmaceutical composition includes the reaction precursors to be used to generate the water-soluble NIR-II fluorescent agent according to the present disclosure. Other packaged compositions or pharmaceutical compositions provided by the present disclosure further include indicia including at least one of: instructions for using the water-soluble NIR-II fluorescent agent to image a subject, or subject samples (e.g., cells or tissues), which can be used as an indicator of conditions including, but not limited to, a disease and biological related events.

Embodiments of this disclosure encompass kits that include, but are not limited to, the water-soluble NIR-II fluorescent agent and directions (written instructions for their use). The kit can further include appropriate buffers and reagents known in the art for administering various combinations of the components listed above to the subject cell or subject organism. The water-soluble NIR-II fluorescent agent and carrier may be provided in solution or in lyophilized form. When the water-soluble NIR-II fluorescent agent and carrier of the kit are in lyophilized form, the kit may optionally contain a sterile and physiologically acceptable reconstitution medium such as water, saline, buffered saline, and the like.

### Dosage Forms

Embodiments of the present disclosure can be included in one or more of the dosage forms mentioned herein. Unit dosage forms of the pharmaceutical compositions (the “composition” includes at least the water-soluble NIR-II fluorescent agent) of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. In an exemplary embodiment, the unit dosage form is given to the subject via intravenous injection.

The amounts and a specific type of active ingredient (e.g., water-soluble NIR-II fluorescent agent) in a dosage form may differ depending on various factors. It will be understood, however, that the total daily usage of the compositions of the present disclosure will be decided by the attending physician or other attending professional within the scope of sound medical judgment. The specific effective dose level for any particular subject will depend upon a variety of factors, including for example, the activity of the specific composition employed; the specific composition employed; the age, body weight, general health, sex, and diet of the subject; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved. Exemplary dosages are provided in the Examples.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Brief Introduction:

Recent work have shown that fluorescence detection in the second near-infrared (NIR) window (NIR-II, 1,000-1,700 nm) with carbon nanotube and quantum dot emitters allows for unprecedented tissue penetration depths for in vivo epifluorescence imaging, affording high spatial and temporal resolution owing to low autofluorescence and reduced photon scattering in the long-wavelength NIR-II spectral range. Here, we develop the first small molecule based NIR-II fluorescence agent for biological imaging in the >1000 nm range. A highly hydrophobic NIR-II fluorescent organic molecule IR-1061 is made water-soluble and biocompatible by embedding in a polymer nanoparticle coated with hydrophilic polyethylene glycol (PEG) chains. The resulting polymer-dye nanoparticles exhibit bright fluorescence emission in the NIR-II window with high photostability in aqueous media. In vivo mice imaging is performed for the first time with organic dyes emitting>1,000 nm to reach deeper penetration depth than traditional NIR imaging in the 750-800 nm window, affording visualization of the inner organs of mice and high resolution images of blood vessels well below the skin of mouse hindlimb.

### Introduction:

Traditional near-infrared (NIR) imaging in the 750-800 nm region has been widely pursued for biological research and biomedical applications in the past decades. It is well known NIR imaging can benefit from reduced optical absorption and lower autofluorescence of biological substances in this ‘biological transparency window’ than in the visible range (1, 2). Nowadays, many fluorophores exist in the visible range and an increasing number of NIR fluorophores have become available commercially, as a result of decades of fluorophore research (3-6) including the first NIR fluorophore indocyanine green (ICG) approved by the US Food and Drug Administration (FDA) for human use. ICG is a water-soluble tricarbocyanine dye emitting at ˜800 nm in the NIR region and can be safely intravenously administered in human (7).

In recent years, we and others have found that fluorescent agents with longer wavelength emission in a new NIR-II window (1.0-1.7 μm) are advantageous for biological imaging mainly due to much reduced tissue scattering of photons in this range (8-20). Prior to these efforts, the NIR-II window has been rarely explored for biological imaging since no biocompatible fluorophores exist emitting>1000 nm and that a ˜1000 nm borderline exists separating commonly used Si photodetectors and cameras from the lower bandgap semiconductor counterparts. The benefit of significantly reduced photon scattering overweighs the disadvantage of slightly increased light absorption in the NIR-II window, making it possible for in vivo fluorescent imaging to achieve higher spatial resolution at much greater penetration depths (several millimeters) than traditional NIR fluorescence imaging (<1 mm) with whole animals (11, 25-31)).

Inorganic carbon nanotubes (8-15), several quantum dots (e.g., Ag2S, Ag2Se, InSb) (16-19) and rare earth nanoparticles (20) are the only fluorescent agents used for biological imaging in the NIR-II region thus far. It will be desirable to develop or synthesize small organic molecules emitting>1,000 nm to build a much larger library of NIR-II fluorophores as in the visible and traditional NIR windows. Small organic molecule approaches are most likely to eventually satisfy all of the desired characteristics of NIR-II fluorophores including high quantum yield, high biocompatibility and rapid excretion from animals and humans. Currently, there are only a small number of organic molecules known to fluoresce in the >1000 nm NIR-II region, all of which are highly hydrophobic, water insoluble cyanine or thiopyrilium dyes including IR-26, IR-1048, IR-1051 and IR-1061 (21-24). None of the organic dyes in NIR-II have been used or are compatible in their native forms for biological imaging.

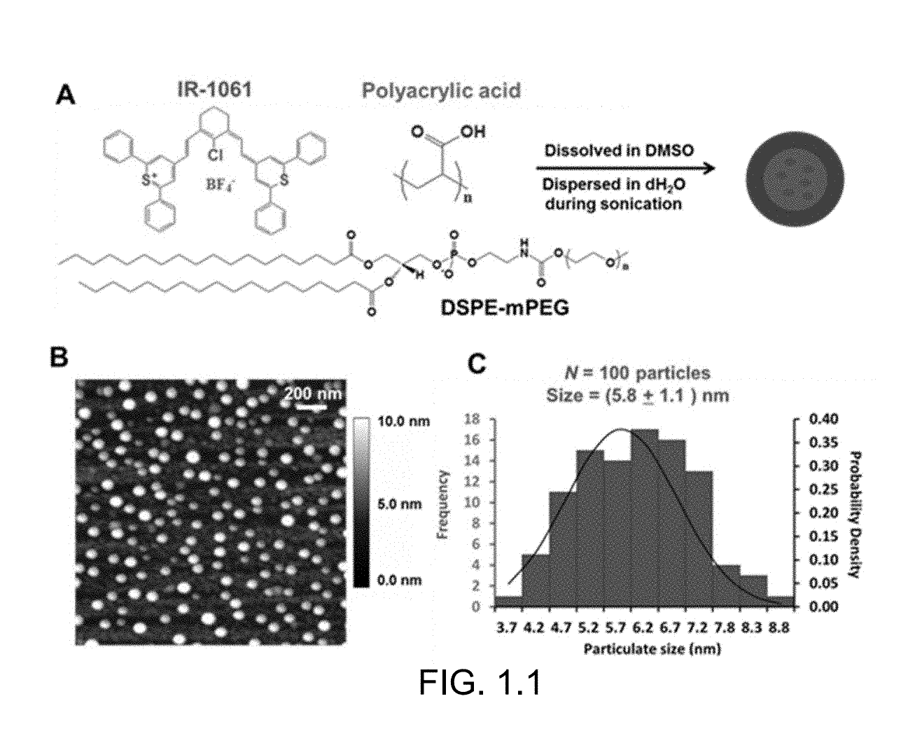

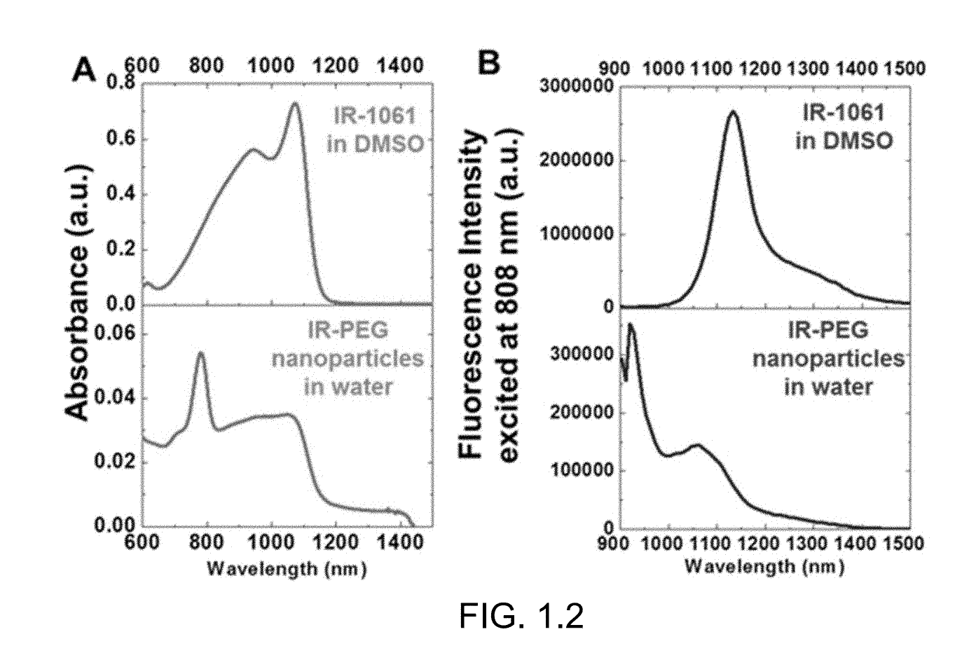

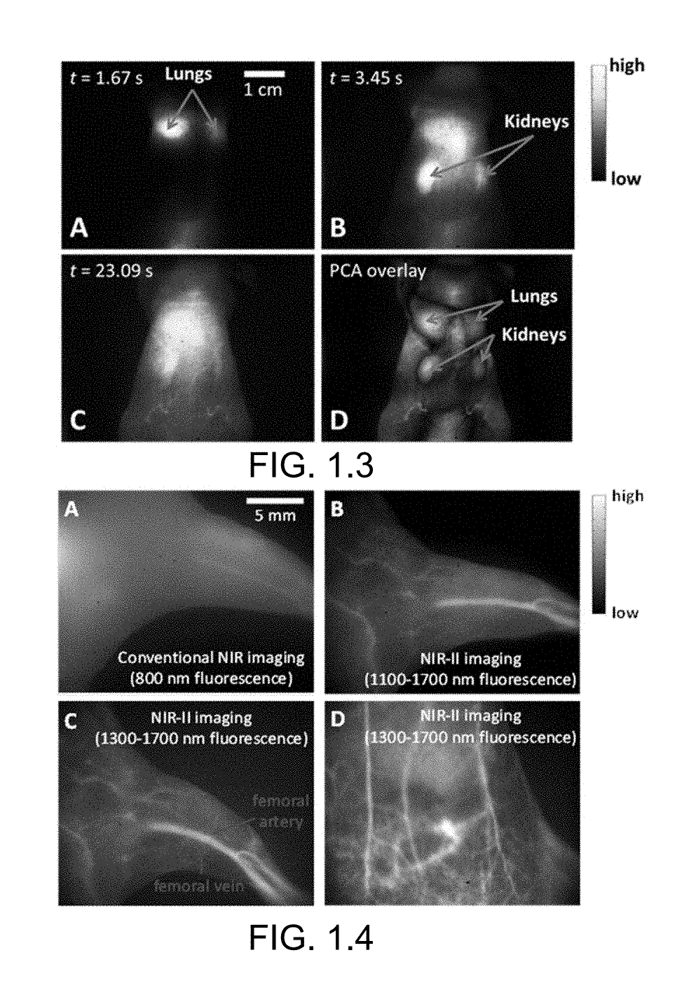

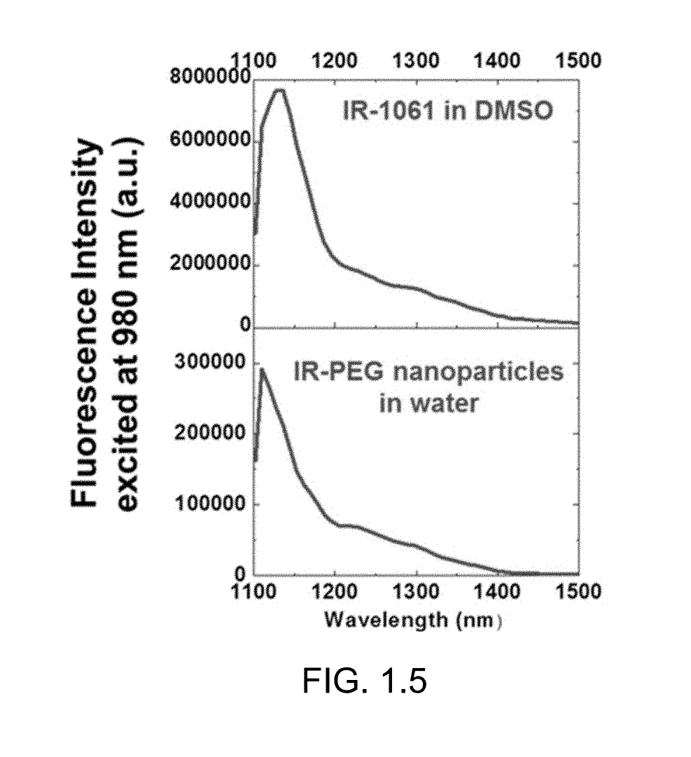

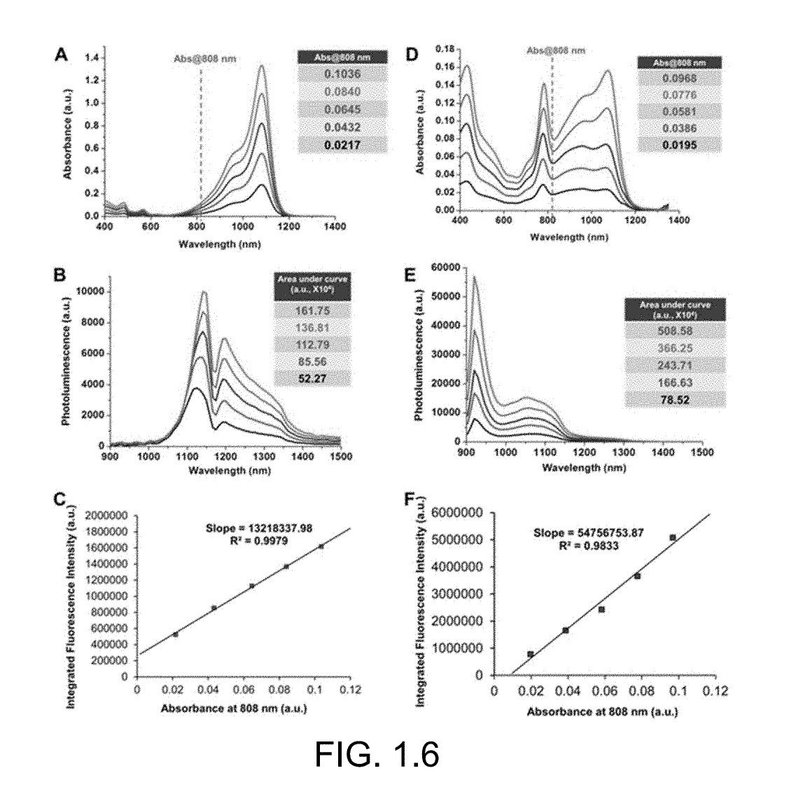

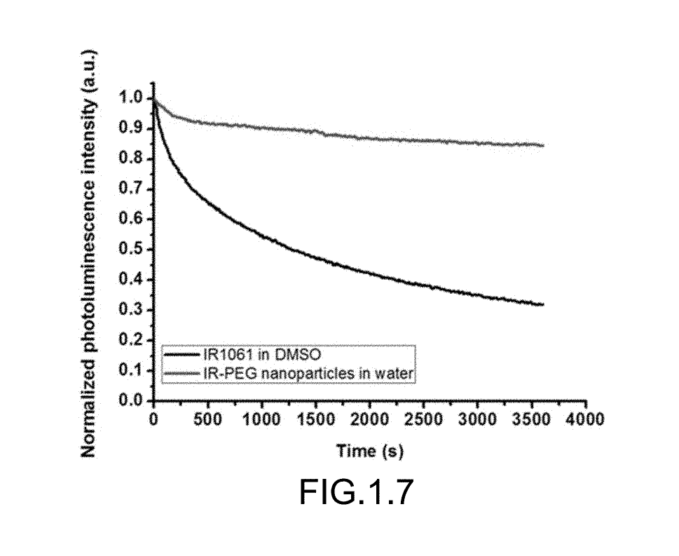

Here we report synthesis of the first biocompatible NIR-II agent based on an organic dye such as for example IR-1061 for in vivo biological imaging. IR-1061 is a commercially available water insoluble polymethine dye with a chemical name of 4-[2-[2-Chloro-3-[(2,6-diphenyl-4H-thiopyran-4-ylidene)ethylidene]-1-cyclohexen-1-yl]ethenyl]-2,6-diphenylthiopyrylium tetrafluoroborate (FIG. 1.1A). IR-1061 is only soluble in several organic solvents including dichloromethane (DCM) and 1, 2-dichloroethane (DCE). Note that the fluorescence quantum efficiency of IR-1061 is not well documented, reported by one study to be ˜(1.7±0.5) % in DCM using rhodamine B in ethanol as a reference (emission below 800 nm) (24). To make IR-1061 water soluble, we mixed the molecules with an amphiphilic polymer such as polyacrylic acid (PAA) (and/or PVA or PEG) to form nanoparticles with IR-1061 embedded in the PAA matrix and then coated the particles with a surfactant N-(carbonyl-methoxypolyethyleneglycol)-1,2-distearoyl-sn-glycero-3-phosphor-ethanolamine (or DSPE-mPEG, i.e., polyethylene glycol-conjugated phospholipid, M.W. ˜5 kDa, FIG. 1.1A).

### Results and Discussions

### Synthesis, Characterization and Photostability of PEGylated IR-1061-Polymer (IR-PEG) Nanoparticles.

We prepared the IR-PEG nanoparticles through a simple one-pot chemical synthesis method. Briefly, IR-1061 and PAA were first dissolved in dimethyl sulfoxide (DMSO) and then quickly added into a DSPE-mPEG aqueous solution, followed by sonication (see Methods for details). DMSO and other excess unreacted reagents were removed from the mixture by centrifugal filtration (100 kDa molecular weight cut-off), and the IR-PEG nanoparticles were resuspended in water, repeatedly washed and centrifuged to remove any precipitates (see Methods).

Atomic force microscopy (AFM) was employed to image the IR-PEG nanoparticles deposited on a silicon substrate. Uniform nanoparticles were observed (FIG. 1.1B), and histogram of the topographic height of the nanoparticles (FIG. 1.1C) showed a median diameter of ˜5.8 nm with a standard deviation of 1.1 nm. The mass ratio of all reactants was IR-1061:PAA:DSPE-mPEG=0.08:1:20 (molar ratio 1.1:5:40). Excess surfactant was removed by centrifugal filtration and ˜54% of DSPE-mPEG remained in the IR-PEG nanoparticles.

The formation of surfactant coated IR-PEG nanoparticles occurred when PAA and IR-1061 dissolved in DMSO were rapidly dispersed into water containing the phospholipid-PEG surfactant. As DMSO mixed with water, the hydrophobic IR-1061 molecules became bound and embedded into the hydrophobic parts of the PAA chains to avoid the aqueous phase, leading to the formation of nanoparticles that were further wrapped by amphiphilic polymers to form an aqueous stable complex containing the hydrophilic arm of PEG chains extended into the water surroundings. PAA has been reported to be an excellent polydentate polymer for functionalization of nanomaterials and is highly biocompatible (32-34). The approach of non-covalent coating of nanoparticles including carbon nanotubes by PEGylated phospholipid by our group and others has been proven highly effective in stabilizing nanoparticles in aqueous solutions including sera and blood, reducing non-specific binding of biomolecules (e.g., proteins) and imparting biocompatibility for in vitro and in vivo use (8-11, 13-17, 35).

Free IR-1061 molecules dissolved in DMSO showed an absorbance peak at 1074 nm and a shoulder peak at 945 nm. The IR-PEG nanoparticles in water exhibited blue-shifted absorbance with a sharp peak at 780 nm and a shoulder peak at 1047 nm, corresponding to change in the local environment of the IR-1061 molecules in the PAA matrix from that in DMSO. The blue shift in the absorbance for IR-PEG nanoparticles in water brought the resonance absorption closer to the 808-nm laser line used for NIR-II imaging (8-11, 13-17). Fluorescence emission spectra under 808-nm laser excitation (FIG. 1.2B) showed an emission peak of free IR-1061 in DMSO at 1132 nm, while the IR-PEG nanoparticle emission in water was blue-shifted (in accordance with the absorption spectra) with a major peak at 920 nm and a shoulder peak at 1064 nm. Similar results were also observed under 980-nm laser excitation (FIG. 1.5)

The fluorescence quantum yield of IR-PEG nanoparticles in water in the NIR-II region was measured to be ˜1.8% using IR-26 molecules in DCE as a reference (quantum yield of IR-26˜0.5%; see SI and FIG. 1.6 for details of quantum yield measurements) (36, 37). Importantly, the IR-PEG nanoparticles in aqueous solutions exhibited much higher photostability than free IR-1061 in DMSO, retaining 85% emission intensity over continuous laser excitation/illumination for 1 h (see FIG. 1.7). These results suggest successful derivation of an aqueous stable NIR-II fluorescent agent based on small organic molecules.

### Biological Imaging Using IR-PEG Nanoparticles.

To explore the IR-PEG nanoparticles as an NIR-II fluorescent agent for biological imaging, we performed whole-body in vivo imaging of nude mice through intravenous injection of a 200 μl solution of IR-PEG nanoparticles in PBS at an injected dose of ˜0.27 mg/ml (based on the mass of IR-1061 embedded in PAA; or ˜360 μm of IR-1061). We performed video-rate imaging at 8.4 frames per second to take advantage of the relative high fluorescence quantum yield of the IR-PEG nanoparticles. Within ˜4 s of injection, fluorescence of the IR-PEG nanoparticles was observed in the lungs and kidneys of the mouse as they passed through these organs with the blood flow (FIG. 1.3A&B), before entering other parts of the mouse (FIG. 1.3C). Based on the time sequence of the NIR-II fluorescence variance throughout the body, we applied principal component analysis (PCA) to convert the temporal variance of the fluorescence intensity evolution at various locations into spatially resolved components (10, 38). Such PCA analysis grouped image pixels with similar time variance into a distinct principal component assigned with a pseudo color, allowing for facile delineation of different inner organs of the mouse including the lungs, kidneys and skin (FIG. 1.3D).

We zoomed into the various regions of the mouse body and performed imaging at higher magnifications (see Methods). The hindlimb region was imaged in both the 1100-1700 nm NIR-II region and the 1300-1700 nm NIR-II region using different long-pass filters, and compared to an image taken in the traditional NIR-I region (˜800 nm, FIG. 1.4A). The image taken in the traditional ˜800 nm NIR window with IRDye800 showed only blurry features of the mouse hindlimb vasculature, due to a high degree of photon scattering in the short-wavelength NIR-I window (FIG. 1.4A) (11). In strong contrast, NIR-II imaging in both the 1100-1700 nm region and the 1300-1700 nm region afforded much clearer images of the mouse hindlimb blood vessels, including the delineation of the parallel femoral artery and vein located in the middle of the hindlimb (FIG. 1.4B&C). The abdominal region was also imaged in the 1300-1700 nm NIR-II region, where rich blood vessels were observed with the smallest vessels measured to be ˜150 μm (FIG. 1.4D). The NIR-II fluorescence images clearly reveal vascular structures located at >1 mm underneath the skin (11), owing to much reduced scattering of photons with the longer wavelengths since photon scattering in turbid biological tissues scales inversely proportional with wavelength (˜λ^{-w}, w=0.22−1.68) (10). None of the mice injected with IR-PEG nanoparticles and used for NIR-II imaging showed any obvious signs of acute toxicity or healthy problems over ˜2 weeks. Thus, our whole-body video-rate imaging as well as steady-state imaging of mouse hindlimb and abdomen suggested the IR-PEG nanoparticles as a biocompatible agent with fluorescence in the >1000 nm NIR-II region, allowing for in vivo imaging with deeper tissue penetration, higher spatial resolution and better image fidelity than traditional fluorescence imaging in the <900 nm NIR region.

### Conclusion

In this work, we developed the first NIR-II fluorescent imaging agent for biological systems based on small organic fluorophores. Mouse inner organ and blood vessel imaging with the IR-PEG nanoparticles represented the first time organic molecular fluorophores were used for in vivo animal research, matching previous NIR-II results with inorganic carbon nanotubes and quantum dots (10, 11). We demonstrated that entrapment of hydrophobic NIR-II organic dyes into an amphiphilic polymeric matrix could afford improved stability of organic fluorophores in aqueous solutions while largely preserving the fluorescent quantum efficiency and imparting biocompatibility for in vivo research. Much work lies ahead in synthesizing water soluble organic fluorophores with aqueous stability and biocompatibility without the need of polymer entrapment approach. Rational organic synthesis of small molecules with >1000 nm fluorescence emission for NIR-II imaging could potentially obtain much higher quantum efficiencies than existing ones, and tune the excitation and emission wavelengths with control. Such small organic fluorophores could also be rapidly excreted out of the body, giving little toxicity problems. With these advances, it is conceivable to develop NIR-II fluorescent agents for widely spread pre-clinical research and clinical use.

### Methods

### Synthesis of IR-PEG Nanoparticles.

0.1 mg IR-1061 dye (m.w.=749.1; 80% dye content, purchased from Sigma-Aldrich) and 1 mg polyacrylic acid (m.w.=2 kDa; Sigma-Aldrich) were dissolved in 1 ml DMSO, while DSPE-mPEG (m.w.=5 kDa; purchase from Laysan Bio, Inc.) was dissolved in 2 ml water at a concentration of 10 mg/ml DSPE-PEG. 1 ml DMSO solution was quickly dispersed into 2 ml aqueous solution (3 ml total) and sonicated by using cup-horn sonicator (Branson Analog 450 Cell Disruptor, Fisher Scientific; power level 5.5) for continuous 5 min. Diluted into a total of 4 ml mixture by adding water, this mixture was further centrifuged using 100 kDa molecular weight cut-off filter (Amicon) to remove DMSO and unreacted reagents, and washed by water 6 times using the same filter. The complex was finally suspended in 3 ml water, followed by ultracentrifugation (50,000 rpm or 300,000 g for 30 min). Supernatant was thus acquired as solutions of IR-PEG nanoparticles. This aqueous solution can be further concentrated by using a 100 kDa filter to eliminate excess water, and centrifuged at 10,000 g for 5 min to remove any substantial precipitates before in vitro cell staining or in vivo mouse injection.

### NIR Fluorescence Spectroscopy.

NIR fluorescence spectra were taken on a home-built NIR fluorescence spectrometer in the 900-1500 nm region when excited by an 808-nm laser and in the 1100-1500 nm region when excited by a 980-nm laser. The excitation light at 808 nm was provided by an 808-nm diode laser (RMPC lasers) at a total output power of 160 mW and filtered through an 850-nm short-pass filter (Thorlabs), a 1000-nm short-pass filter (Thorlabs), an 1100-nm short-pass filter (Omega) and a 1300-nm short-pass filter (Omega). The excitation light at 980 nm was provided by a 980-nm diode laser (LDX Optronics) at a total output power of 160 mW and filtered through a 1000-nm short-pass filter (Thorlabs), an 1100-nm short-pass filter (Omega) and a 1300-nm short-pass filter (Omega). The excitation laser beam was allowed to pass through the solution sample of either IR-1061 in DMSO or IR-PEG nanoparticles in aqueous solutions in a 1 mm path cuvette (Starna Cells) and the emission was collected with the transmission geometry. A 900-nm long-pass filter (Thorlabs) alone was used to reject the excitation light at 808 nm, and a filter set consisting of a 1050-nm long-pass filter (Thorlabs) and an 1100-nm long-pass filter (Thorlabs) was used to reject the excitation light at 980 nm. The emitted fluorescence after laser line rejection was directed into a spectrometer (Acton SP2300i) equipped with an InGaAs linear array (Princeton OMA-V). The emission spectrum was corrected after raw data acquisition to take into account the detector sensitivity profile and the extinction features of the filters.

References, each of which is incorporated herein by reference
1. Pansare V, Hejazi S, Faenza W, Prud'homme R K (2012) Review of long-wavelength optical and NIR imaging materials: contrast agents, fluorophores and multifunctional nano carriers. Chem Mater 24:812-827.
2. Altino\check{g}lu E I, Adair J H (2010) Near infrared imaging with nanoparticles. Wiley Interdiscip Rev Nanomed Nanobiotechnol 2:461-477.
3. Mujumdar R B, et al. (1993) Cyanine dye labeling reagents: sulfoindocyanine succinimidyl esters. Bioconjug Chem 4:105-111.
4. Lin Y, Weissleder R, Tung C H. (2002) Novel near-infrared cyanine fluorochromes: synthesis, properties, and bioconjugation. Bioconjug Chem 13:605-610.
5. Bouteiller C, et al. (2007) Novel water-soluble near-infrared cyanine dyes: synthesis, spectral properties, and use in the preparation of internally quenched fluorescent probes. Bioconjug Chem 18:1303-1317.
6. Ebert B, Riefke B, Sukowski U, Licha K (2011) Cyanine dyes as contrast agents for near-infrared imaging in vivo: acute tolerance, pharmacokinetics, and fluorescence imaging. J Biomed Opt 16:066003.
7. Schaafsma B E, et al. (2011) The clinical use of indocyanine green as a near-infrared fluorescent contrast agent for image-guided oncologic surgery. J Surg Oncol 104:323-332.
8. Welsher K, et al. (2009) A route to brightly fluorescent carbon nanotubes for near-infrared imaging in mice. Nat Nanotechnol 4:773-780.
9. Robinson J T, et al. (2010) High performance in vivo near-IR (>1 μm) imaging and photothermal cancer therapy with carbon nanotubes. Nano Res 3:779-793.
10. Welsher K, Sherlock S P, Dai H (2011) Deep-tissue anatomical imaging of mice using carbon nanotube fluorophores in the second near-infrared window. Proc Natl Acad Sci USA 108:8943-8948.
11. Hong G, et al. (2012) Multifunctional in vivo vascular imaging using near-infrared II fluorescence. Nat Med 18:1841-1846.
12. Yi H, et al. (2012) M13 phage-functionalized single-walled carbon nanotubes as nanoprobes for second near-infrared window fluorescence imaging of targeted tumors. Nano Lett 12:1176-1183.
13. Robinson J T, et al. (2012) In vivo fluorescence imaging in the second near-infrared window with long circulating carbon nanotubes capable of ultrahigh tumor uptake. J Am Chem Soc 134:10664-10669.
14. Diao S, et al. (2012) Chirality enriched (12,1) and (11,3) single-walled carbon nanotubes for biological imaging. J Am Chem Soc 134:16971-16974.
15. Antaris A L, et al. (2013) Ultra-low doses of chirality sorted (6,5) carbon nanotubes for simultaneous tumor imaging and photothermal therapy. ACS Nano 7:3644-3652.
16. Hong G, et al. (2012) In vivo fluorescence imaging with Ag2S quantum dots in the second near-infrared region. Angew Chem Int Ed Engl 51:9818-9821.
17. Zhang Y, et al. (2012) Ag2S quantum dot: a bright and biocompatible fluorescent nanoprobe in the second near-infrared window. ACS Nano 6:3695-3702.
18. Liu W, Chang A Y, Schaller R D, Talapin D V (2012) Colloidal InSb nanocrystals. J Am Chem Soc 134:20258-20261.
19. Dong B, et al. (2013) Facile synthesis of highly photoluminescent Ag2Se quantum dots as a new fluorescent probe in the second near-infrared window for in vivo imaging. Chem Mater 25: 2503-2509.
20. Naczynski D J, et al. (2013) Rare-earth-doped biological composites as in vivo shortwave infrared reporters. Nat Commun 4: 2199.
21. Kanofsky J R, Sima P D (2000) Structural and environmental requirements for quenching of singlet oxygen by cyanine dyes. Photochem Photobiol 71:361-368.
22. Prosposito P, et al. (2001) Femtosecond dynamics of IR-molecules in hybrid materials. Journal of Luminescence 94-95: 641-644.
23. Prosposito P, et al. (2003) IR-luminescent molecules in hybrid materials. Journal of Sol-Gel Science and Technology 26: 909-913.
24. Casalboni M, et al. (2003) Fluorescence efficiency of four infrared polymethine dyes. Chemical Physics Letters 373: 372-378.
25. Farmer S C, Patten T E (2001) Photoluminescent polymer/quantum dot composite nanoparticles. Chem Mater 13: 3920-3926.
26. Li K, et al. (2009) Generic strategy of preparing fluorescent conjugated-polymer-loaded poly(DL-lactide-co-glycolide) nanoparticles for targeted cell imaging. Advanced Functional Materials 19: 3535-3542.
27. Park K, Lee S, Kang E, Kim K, Choi K, Kwon I C (2009) New generation of multifunctional nanoparticles for cancer imaging and therapy. Advanced Functional Materials 19: 1553-1566.
28. Acharya S, Sahoo S K (2011) PLGA nanoparticles containing various anticancer agents and tumour delivery by EPR effect. Adv Drug Deliv Rev 63:170-183.
29. Wang X, Xu S, Xu W (2011) Synthesis of highly stable fluorescent Ag nanocluster polymer nanoparticles in aqueous solution. Nanoscale 3: 4670-4675.
30. Dossi M, et al. (2013) Synthesis of fluorescent PMMA-based nanoparticles. Macromolecular Materials and Engineering 298: 771-778.
31. Jin Y, Ye F, Zeigler M, Wu C, Chiu D T (2011) Near-infrared fluorescent dye-doped semiconducting polymer dots. ACS Nano 5:1468-1475.
32. Hilderbrand S A, et al. (2009) Upconverting luminescent nanomaterials: application to in vivo bioimaging. Chem Commun 4188-4190.
33. Ke J H, et al. (2010) A specific tumor-targeting magnetofluorescent nanoprobe for dual-modality molecular imaging. Biomaterials 31:1707-1715.
34. Xiong L, et al. (2010) Long-term in vivo biodistribution imaging and toxicity of polyacrylic acid-coated upconversion nanophosphors. Biomaterials 31:7078-7085.
35. Liu Z, et al. (2007) In vivo biodistribution and highly efficient tumor targeting of carbon nanotubes in mice. Nat Nanotechnol 2:47-52.
36. Murphy J E, et al. (2006) PbTe colloidal nanocrystals: synthesis, characterization, and multiple exciton generation. J Am Chem Soc 128:3241-3247.
37. Semonin O E, et al. (2010) Absolute photoluminescence quantum yields of IR-26 dye, PbS, and PbSe quantum dots. J Phys Chem Lett 1:2445-2450.
38. Hillman E M, Moore A (2007) All-optical anatomical co-registration for molecular imaging of small animals using dynamic contrast. Nat Photonics 1:526-530.

### Example 1 Supplemental Information

### UV-Vis-NIR Absorption Spectrum Measurements.

UV-Vis-NIR absorption spectra of different solutions as mentioned in the study were measured by using a Cary 6000i UV-Vis-NIR spectrophotometer, background-corrected for contribution from the employed solvents. The wavelength range was selected to be 600-1500 nm, and the solution sample was placed in a 1-mm quartz rectangular spectrophotometer cuvette (Starna Cells, 1 mm path length, 0.4 ml capacity).

Estimation of Residual Amount of Surfactant and Dye after Nanoparticle Synthesis.

In a typical synthesis of IR-PEG nanoparticles, every 3 ml reaction mixture (2 ml water mixed with 1 ml DMSO) contained 0.1 mg IR-1061, 1 mg PAA, and 20 mg DSPE-mPEG, where DSPE-mPEG constituted an overwhelming majority of the nanoparticles in weight. After the formation of nanoparticles in water, the solutions were immediately frozen by liquid nitrogen and sent for lyophilization overnight. The lyophilized powder was weighed and assumed to containing only DSPE-mPEG by neglecting a marginal amount of dye and polymer in the samples. The residual percentage of surfactant was calculated as follows:

$$\frac{surfactant remaining in the nanoparticles}{surfactant originally applied} \approx \frac{synthesized products after lyophilization}{surfactant originally applied}$$

Estimation of IR-1061 composition in the nanoparticles was carried out as follows: nanoparticles in water after synthesis were lyophilized and re-dissolved in DMSO: DCM (v/v 1:1) mixture. The mixture was investigated using UV-Vis-NIR spectroscopy with scanned range from 300 nm to 1200 nm, and compared to the spectra in the same range obtained from different concentrations of IR-1061 in DMSO: DCM (v/v 1:1) mixture. Absorbance at 438 nm in all spectra, where an absorption peak showed up, was utilized to determine the dye concentration.

### NIR Fluorescence Quantum Yield Measurement of IR-PEG Nanoparticles.

Fluorescence quantum yield of the prepared IR-PEG nanoparticles was measured in a similar manner to a previous publication (1), using the IR-26 dye as the reference with a quantum yield of 0.5% (2, 3). In a typical procedure, a stock solution of 1 mg/mL IR-26 in 1,2-dichloroethane (DCE) was diluted to a solution in DCE with absorbance of around 0.10 at 808 nm. Then a series of samples with decreasing concentrations were made from serial dilution of this solution and UV-Vis-NIR absorption spectra of these samples were taken to confirm their absorbance values at 808 nm were ˜0.08, ˜0.06, ˜0.04 and ˜0.02, respectively (FIG. 1.6A). Then a total of five solutions with linearly spaced concentrations (including the first one with absorbance of ˜0.10 at 808 nm) were loaded into a 10-mm fluorescence cuvette one at a time and their NIR emission spectra were taken in the same setup described in the preceding paragraph. Here only the 808-nm laser was used as the excitation and a 900-nm long-pass filter was used as the emission filter to acquire the emission spectrum in the 900-1500 nm region (FIG. 1.6B). The same absorption and emission measurements were performed for IR-PEG nanoparticles in aqueous solutions too (FIG. 1.6D-E). Then all emission spectra were corrected after raw data acquisition to account for the detector sensitivity profile and the extinction features of the filters, and integrated in the 900-1500 nm region. The integrated fluorescence intensity was plotted against absorbance at 808 nm (the excitation wavelength) and fitted into a linear function (FIG. 1.6C & 1.6F). Two slopes, one obtained from the IR-26 DCE reference and the other from the IR-PEG nanoparticle sample, were used in the calculation of the quantum yield of IR-PEG nanoparticle in water, according to the following equation,

$${QY}_{sample} = {QY}_{ref} \cdot \frac{{slope}_{sample}}{{slope}_{ref}} \cdot {( \frac{{n}_{sample}}{{n}_{ref}} )}^{2}$$

where QY_{ref} is 0.5% and nₛₐₘₚₗₑ and n_{ref} are the refractive index of water and DCE, respectively.

### Mouse Handling and Injection.

Mouse handling and injection procedures are described in the Stanford Institutional Animal Care and Use Committee protocols. Athymic nude mice (n=4), purchased from Charles River, were injected intravenously through the tail with a 200 μl solution of IR-PEG nanoparticles (360 μm IR-1061 in 1×PBS) for video-rate in vivo fluorescence imaging. The mice were anesthetized using 2 L/min of 02 with 2.5% isoflurane during the injection and subsequent imaging. Prior to injection, IR-PEG nanoparticle solutions were ultra-centrifuged at 10,000 g for 5 min to remove aggregates and bacteria. For fluorescence imaging in the NIR-I window, a 200 μl solution of IRDye-800 conjugated to single-walled carbon nanotubes ([IRDye-800]=33.3 μm) was made by conjugating the IRDye-800 NHS ester (LI-COR) to amine-functionalized nanotubes and injected into the mouse tail vein.

### NIR Fluorescence Imaging of Live Mice.

A liquid-nitrogen-cooled, 320×256 pixel two-dimensional InGaAs array (Princeton Instruments) was used to capture fluorescence images of IR-1061 solutions in the NIR-II region. The excitation light was provided by an 808-nm diode laser (RMPC lasers) and filtered by an 850-nm short-pass filter and a 1000-nm short-pass filter (Thorlabs) before reaching the animal on the imaging plane with an excitation power density of 140 mW·cm⁻². The emitted epifluorescence from the animal was allowed to pass through a 900-nm long-pass filter and an 1100-nm long-pass filter (Thorlabs) to collect fluorescence in the >1100 nm NIR-II region. A lens pair consisting of two touching achromats (200 mm and 75 mm, Thorlabs) was used to focus the fluorescence image of the entire mouse body onto the detector. To record a video showing the circulation of injected IR-PEG nanoparticles in the whole body, the InGaAs camera was set to expose continuously, with an exposure time of 100 ms for each image acquisition. There was also an 19-ms overhead time in the readout, leading to a total time of 119 ms between consecutive frames and a frame rate of 8.4 frames·s⁻¹ for the video.

For stationary imaging of the hindlimb and abdomen areas of the mouse after injection, the distance of the two aforementioned achromats (200 mm and 75 mm, Thorlabs) was adjusted to afford a magnification of 2.5× higher than the whole body imaging setup. The field of view was 25 mm×20 mm, covering only the hindlimb or the abdomen area. For NIR-I fluorescence imaging under this magnification, the excitation light was provided by a 785-nm diode laser (Renishaw) and filtered through a 790-nm bandpass filter (Thorlabs). The emitted fluorescence from the mouse in the front-face geometry was allowed to pass through a 790-nm longpass filter (Semrock) and an 850-nm shortpass filter (Thorlabs) to collect fluorescence in the NIR-I region. For NIR-II fluorescence imaging under this magnification, the excitation light was provided by an 980-nm diode laser (LDX Optronics) and filtered through a 1000-nm short-pass filter (Thorlabs) before reaching the mouse on the imaging plane with an excitation power density of 600 mW·cm⁻², lower than the safe exposure limit of 726 mW·cm⁻² at 980 nm (4). The emitted fluorescence from the mouse in the front-face geometry was allowed to pass through a 1000-nm long-pass filter and an 1100-nm long-pass filter (Thorlabs) to collect fluorescence in the >1100 nm NIR-II region with an exposure time of 150 ms, or a 1000-nm long-pass filter and an 1300-nm long-pass filter (Thorlabs) to collect fluorescence in the >1300 nm NIR-IIa region with an exposure time of 300 ms.

### Dynamic Contrast-Enhanced Imaging Based on PCA.

Dynamic contrast-enhanced images were obtained in a similar way to previous work by the Hillman group (5) and our group (6). First 200 consecutive frames immediately after injection from the video-rate whole body imaging were loaded into an array using MATLAB software, and the built-in princomp function was used to perform PCA. The overlaid image includes positive and negative 2^{nd}, 3^{rd} and 4^{th} components, color-coded in red, green and blue, respectively.

### References for Example 1 Supplemental Information

1. Williams A T R, Winfield S A, Miller, J N (1983) Relative fluorescence quantum yields using a computer-controlled luminescence spectrometer. Analyst 108: 1067-1071.
2. Murphy J E, et al. (2006) PbTe colloidal nanocrystals: synthesis, characterization, and multiple exciton generation. J Am Chem Soc 128:3241-3247.
3. Semonin O E, et al. (2010) Absolute photoluminescence quantum yields of IR-26 dye, PbS, and PbSe quantum dots. J Phys Chem Lett 1:2445-2450.
4. Matthes R, et al. (2000) Revision of guidelines on limits of exposure to laser radiation of wavelengths between 400 nm and 1.4 μm. Health Phys 79: 431-440.
5. Hillman E M, Moore A (2007) All-optical anatomical co-registration for molecular imaging of small animals using dynamic contrast. Nat Photonics 1:526-530.
6. Welsher K, Sherlock S P, Dai H (2011) Deep-tissue anatomical imaging of mice using carbon nanotube fluorophores in the second near-infrared window. Proc Natl Acad Sci USA 108:8943-8948.

### Example 2

### Brief Introduction:

In vivo fluorescence imaging in the second near-infrared window (1.0-1.7 μm) can afford deep tissue penetration and high spatial resolution, owing to the reduced scattering of long-wavelength photons. Here, we synthesize a series of low-bandgap donor/acceptor copolymers with tunable emission wavelengths of 1050-1350 nm in this window. Non-covalent functionalization with phospholipid-polyethylene glycol results in water-soluble and biocompatible polymeric nanoparticles, allowing for live cell molecular imaging at >1000 nm with polymer fluorophores for the first time. Importantly, the high quantum yield of the polymer allows for in vivo, deep-tissue and ultrafast imaging of mouse arterial blood flow with an unprecedented frame rate of >25 frames per second. The high time resolution results in spatially and time resolved imaging of the blood flow pattern in cardiogram waveform over a single cardiac cycle (˜200 ms) of a mouse, which has not been observed with fluorescence imaging in this window before.

### Introduction:

In vivo biological imaging in the second near-infrared window (NIR-II, 1.0-1.7 μm) has attracted much interest recently′ owing to salient advantages over imaging in the visible (400-750 nm) and the conventional near-infrared (NIR, 750-900 nm) regions. Detecting longer wavelength photons in the NIR-II affords reduced photon scattering in biological tissues accompanied by lower autofluorescence, leading to higher spatial resolution at deeper tissue penetration depths.⁹ To date, single-walled carbon nanotubes (SWNTs)^{1-3,10-14}, semiconducting quantum dots (QDs)^{6,7,15-17}, rare-earth doped nanoparticles⁸ and nanoparticles made of small organic molecules¹⁸ are fluorophores used for fluorescence imaging in the NIR-II region. These NIR-II agents can be functionalized with hydrophilic molecules to afford biocompatibility for intravenous administration and can be gradually cleared out from the body without obvious acute or chronic toxicity in animal studies.^{16,19,20} However, shortcomings of existing NIR-II fluorophores include relatively low fluorescence quantum yield^{1,8,21}, unfavorably short emission wavelength¹⁸ and potential toxicity due to heavy metals in QDs.^{6,7,15-17} A much wider range of fluorophores could be developed to tackle these problems. An ideal fluorophore developed for NIR-II biological imaging should allow for tunable excitation and emission wavelengths in the >1000 nm NIR-II region with a high fluorescence quantum yield and high biocompatibility.

Here we report the synthesis of conjugated polymers with intrinsic fluorescence>1000 nm through donor-acceptor (D-A) alternating copolymerization, an effective way of synthesizing polymers with tunable bandgap energy in the NIR and at even longer wavelength regions.²²⁻²⁹ By copolymerization of an electron-donating monomer benzo[1,2-b:3,4-b′]difuran and an electron-withdrawing monomer fluorothieno-[3,4-b]thiophene, we derive a brightly fluorescent copolymer, poly(benzo[1,2-b:3,4-b′]difuran-alt-fluorothieno-[3,4-b]thiophene) (named ‘pDA’) with fluorescence emission at 1050 nm with a large Stokes shift of ˜400 nm and a high fluorescence quantum yield of ˜1.7%, much higher than the quantum yield of typical carbon nanotubes (˜0.4%) used previously.³ We non-covalently functionalize the polymer with a PEGylated surfactant to afford water solubility and biocompatibility. We successfully tune the emission wavelength of the conjugated polymer fluorophores from 1050 nm to 1350 nm, and demonstrate the first in vitro and in vivo biological imaging in the >1000 nm window using conjugated polymer fluorophores.

### Results:

Synthesis of NIR-II Fluorescent pDA-PEG Nanoparticles.

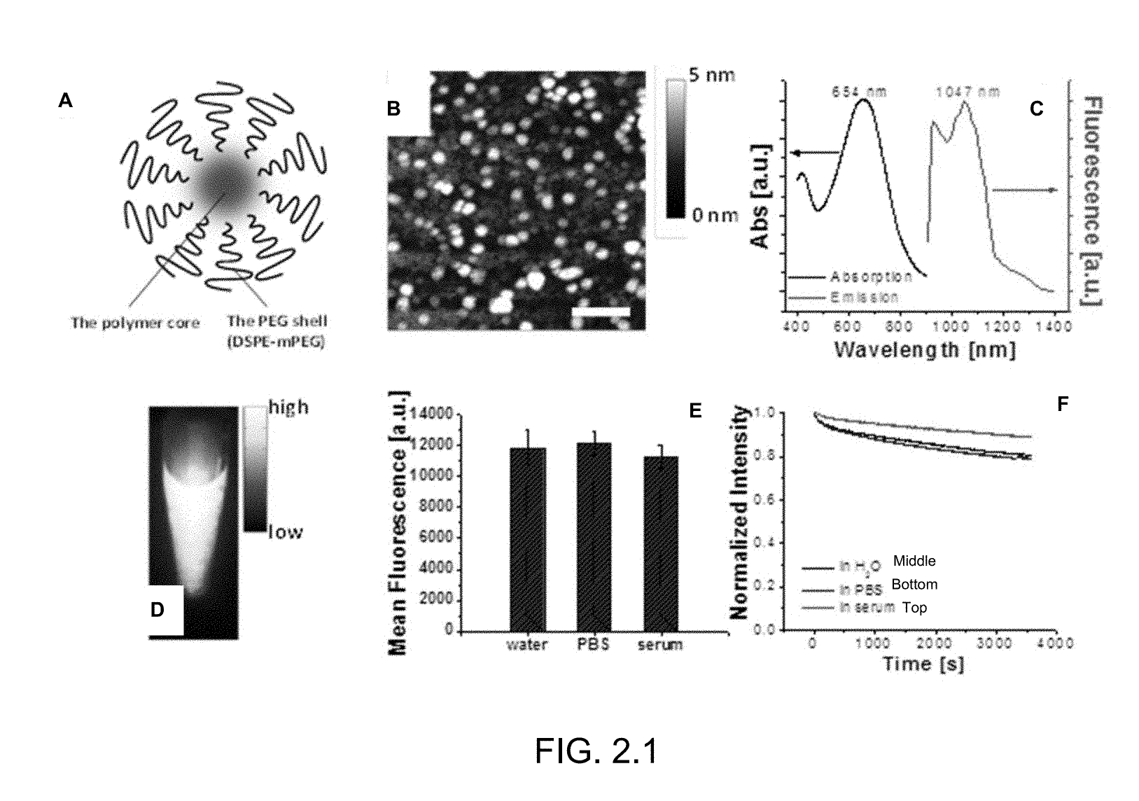

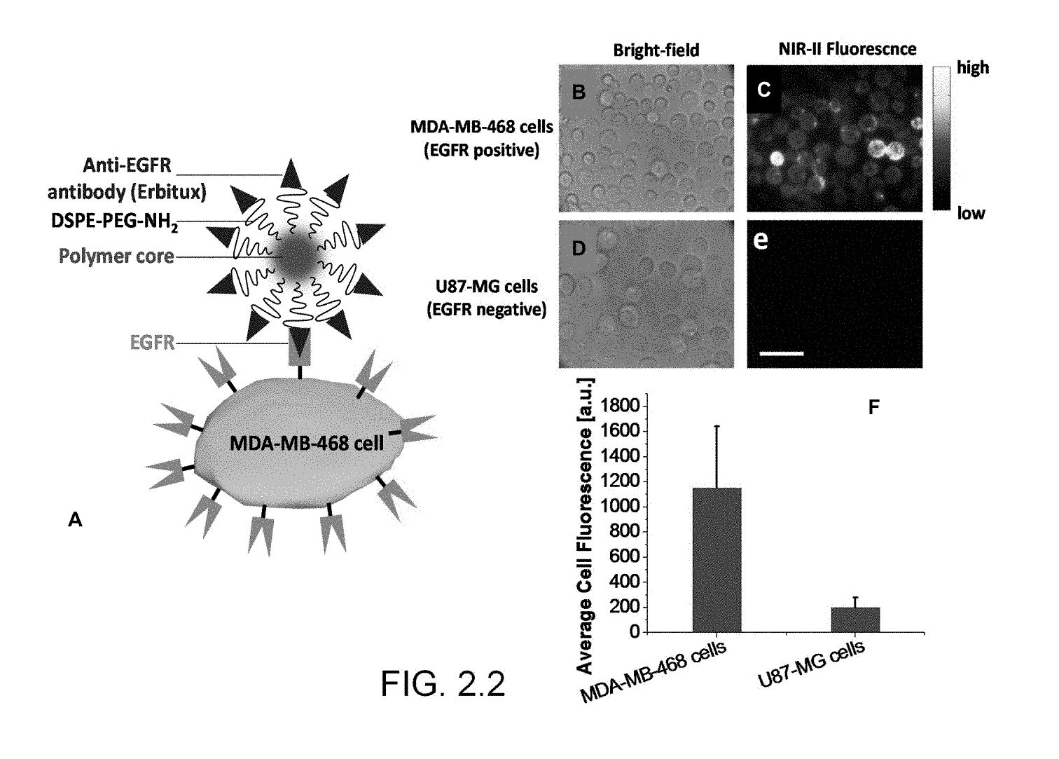

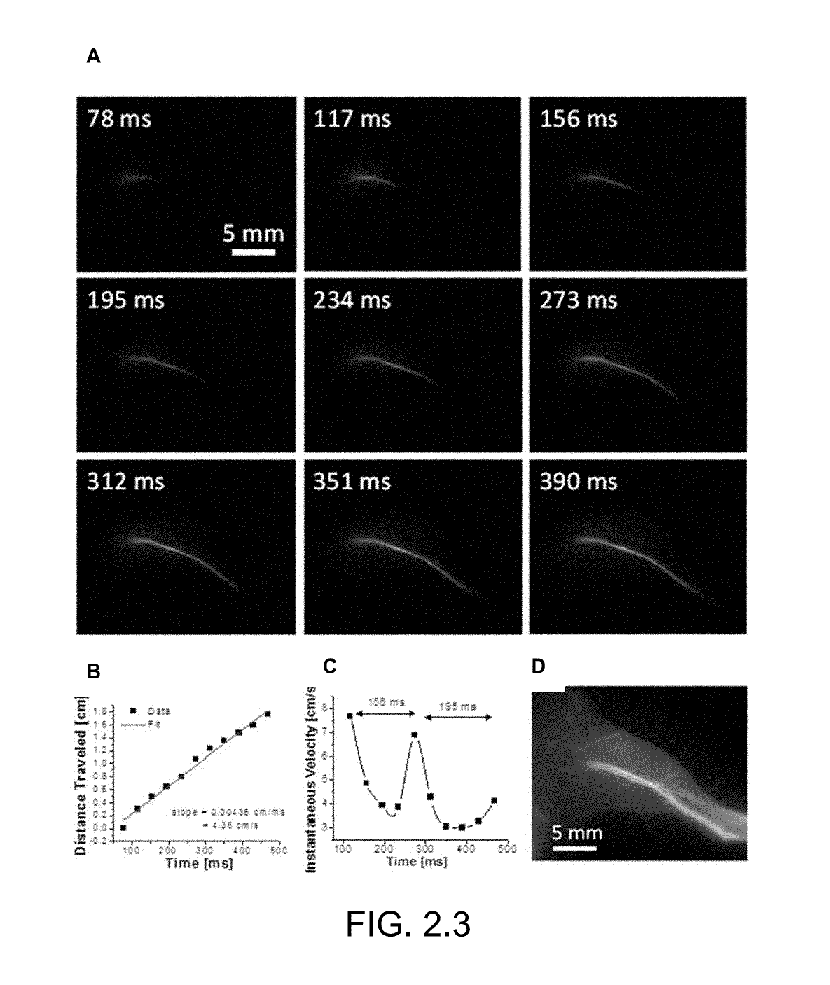

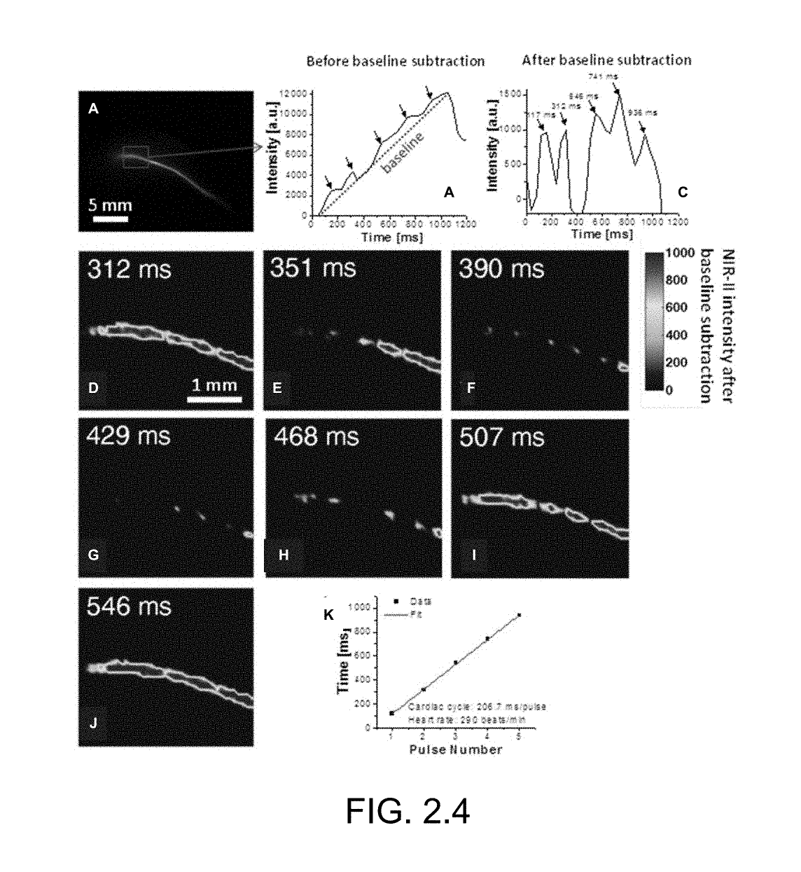

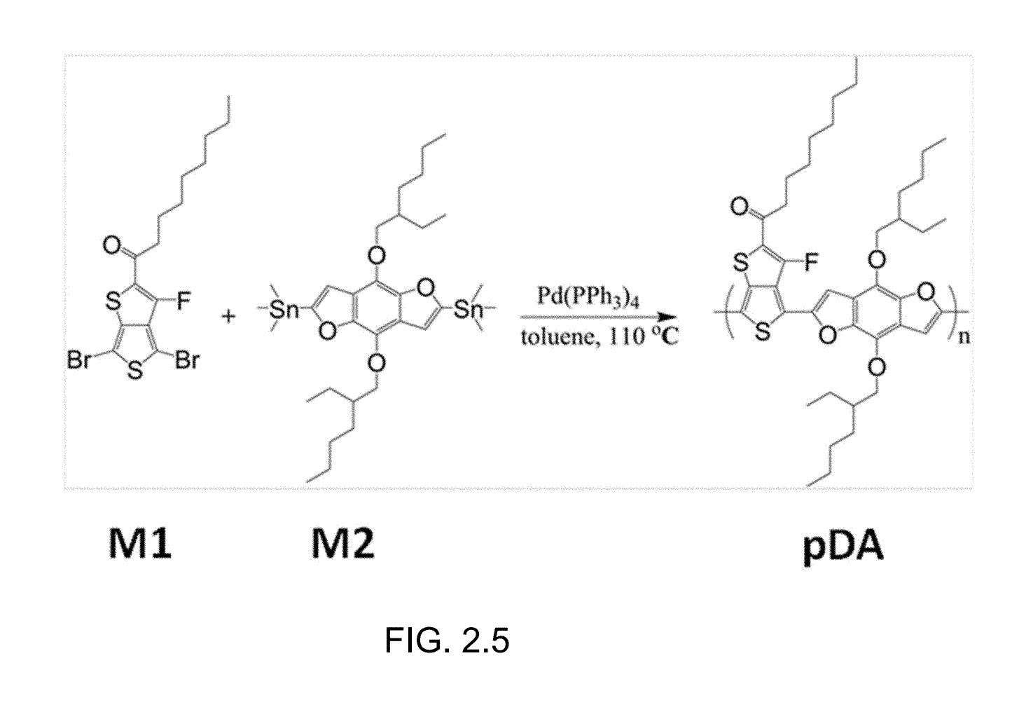

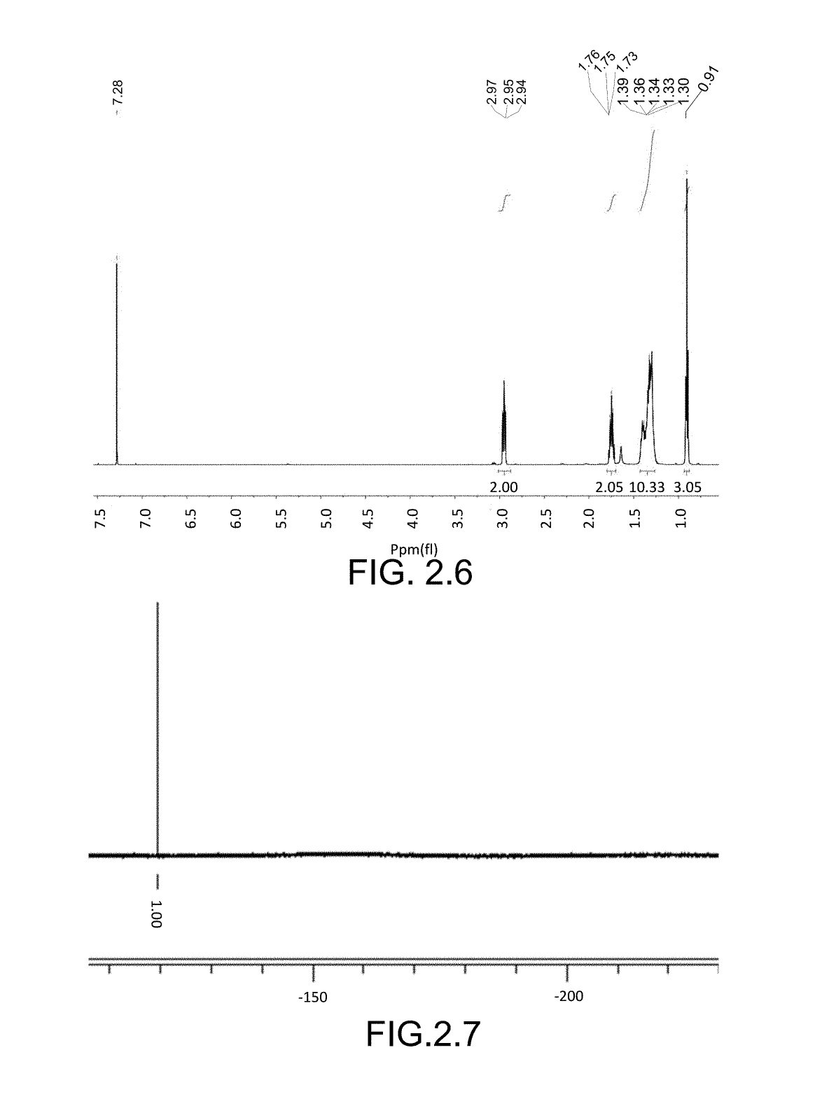

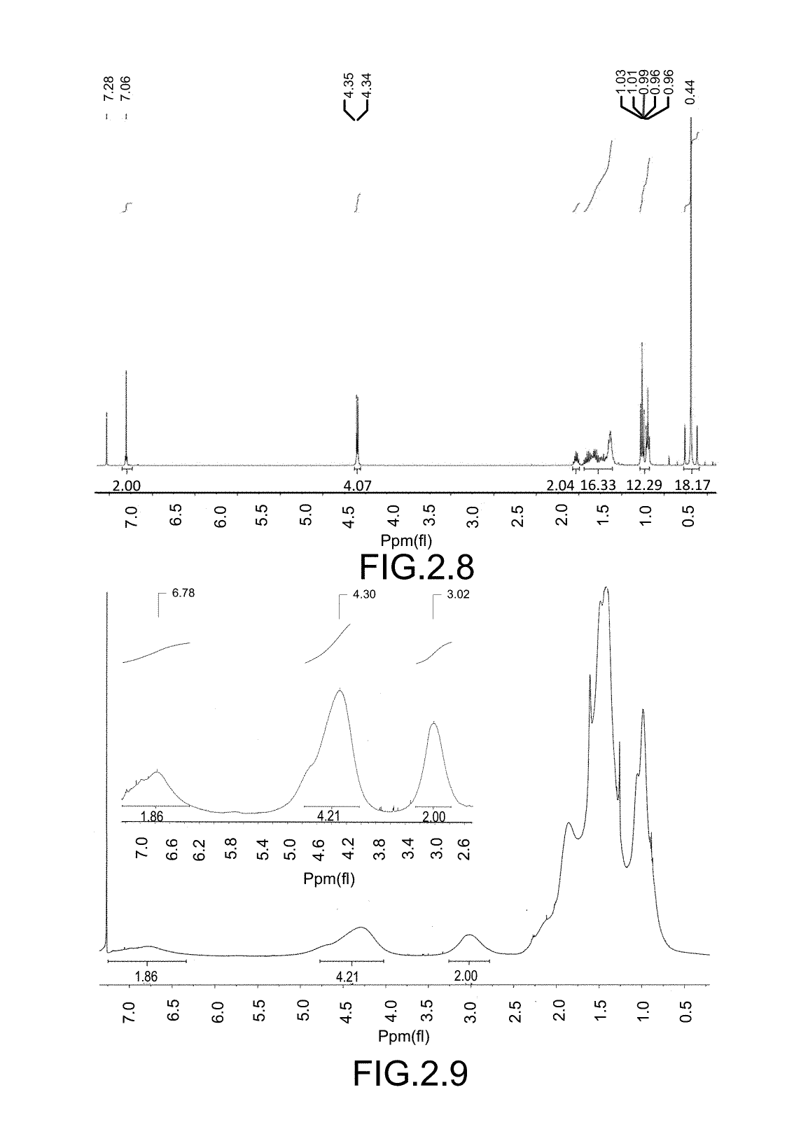

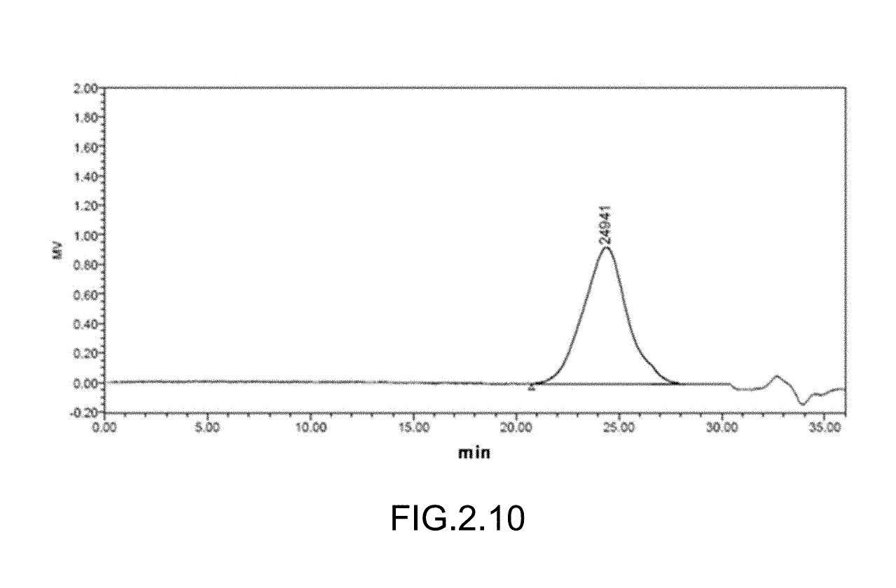

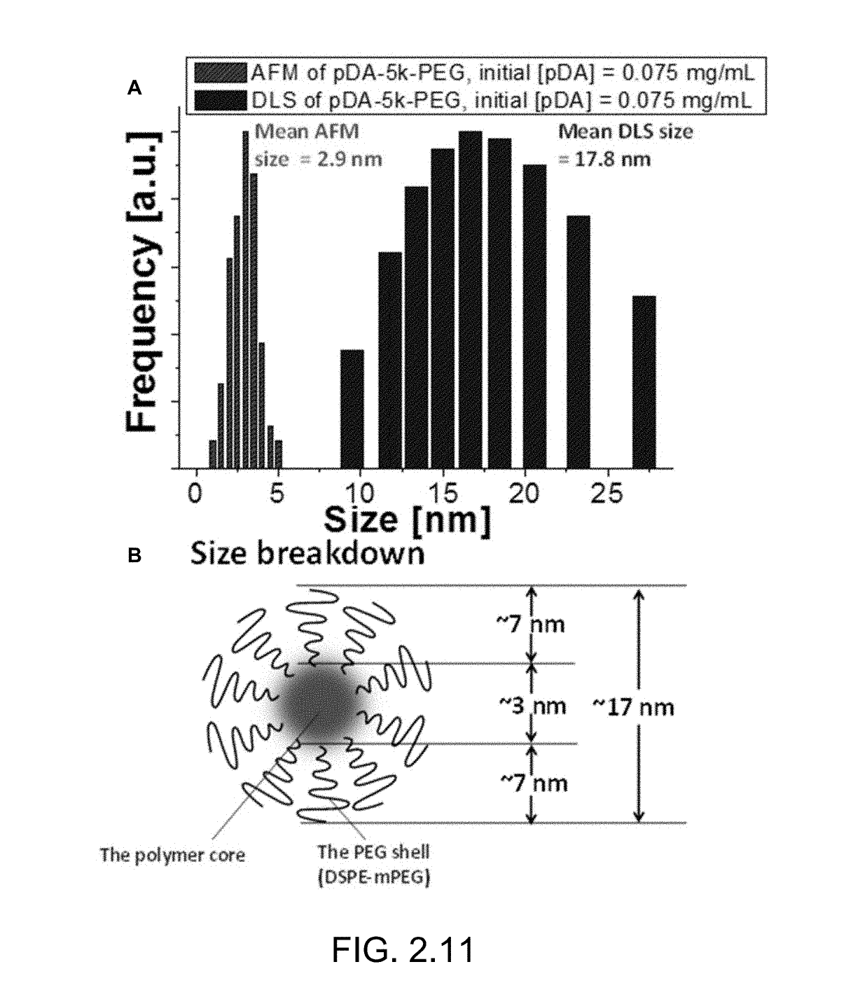

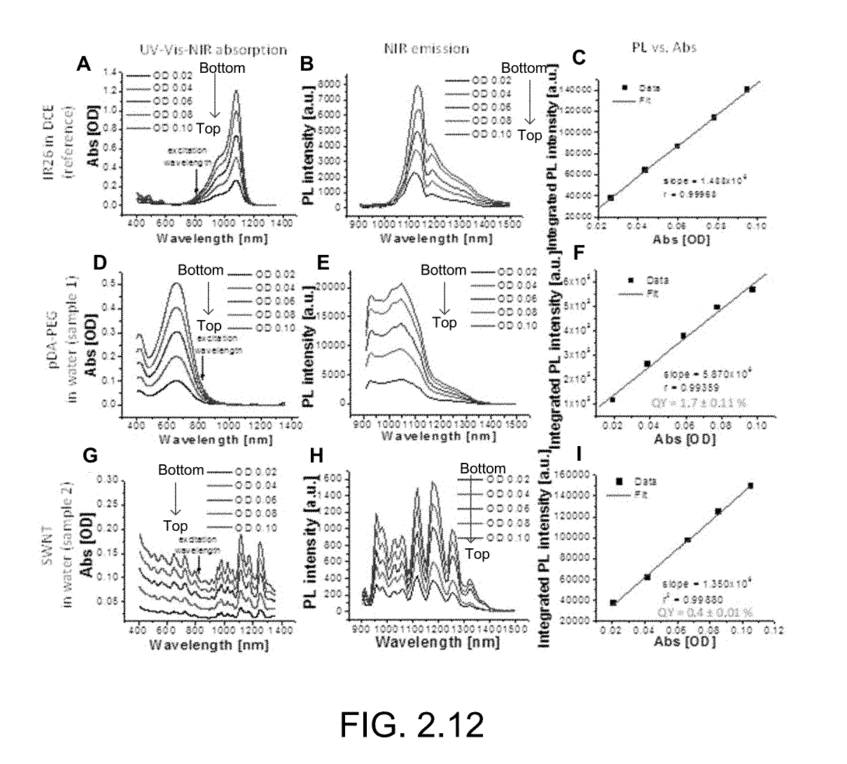

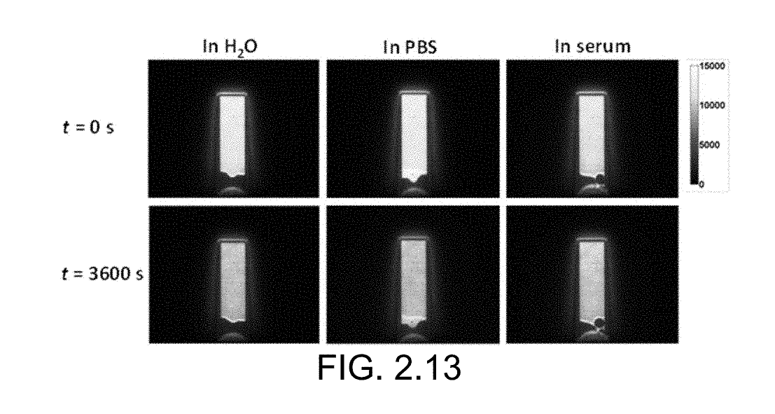

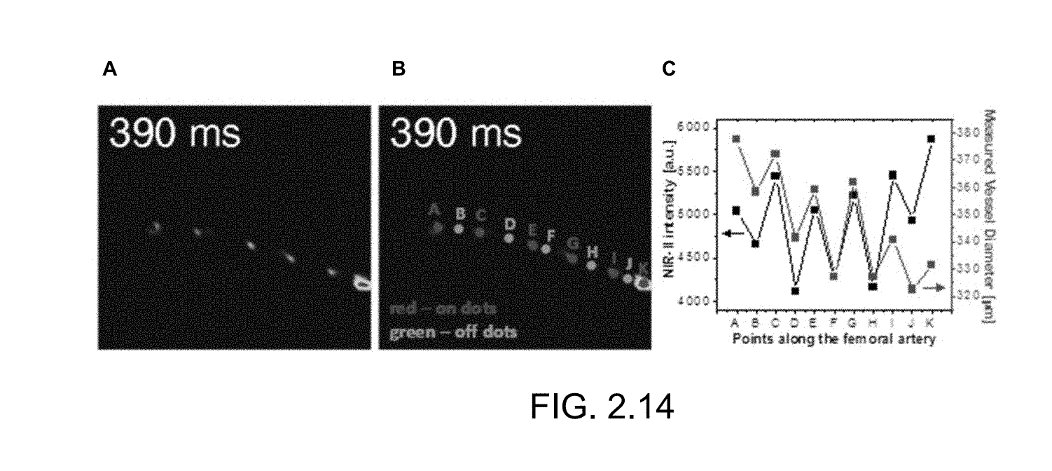

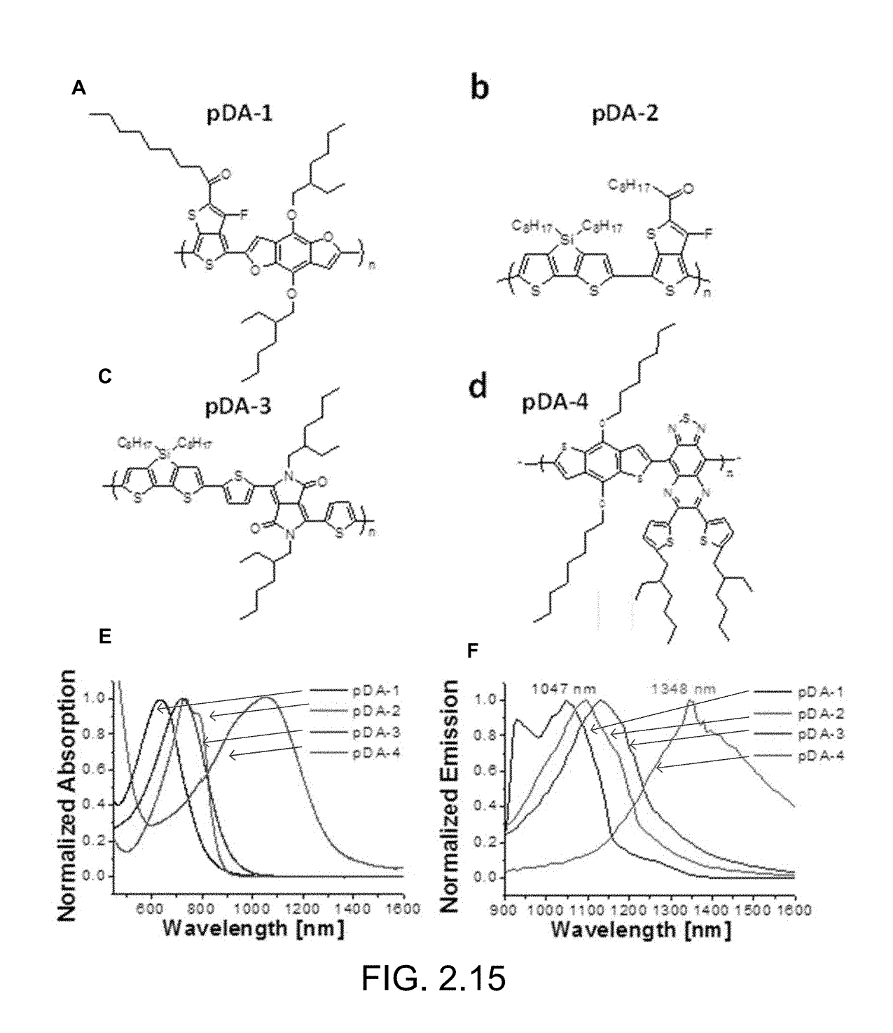

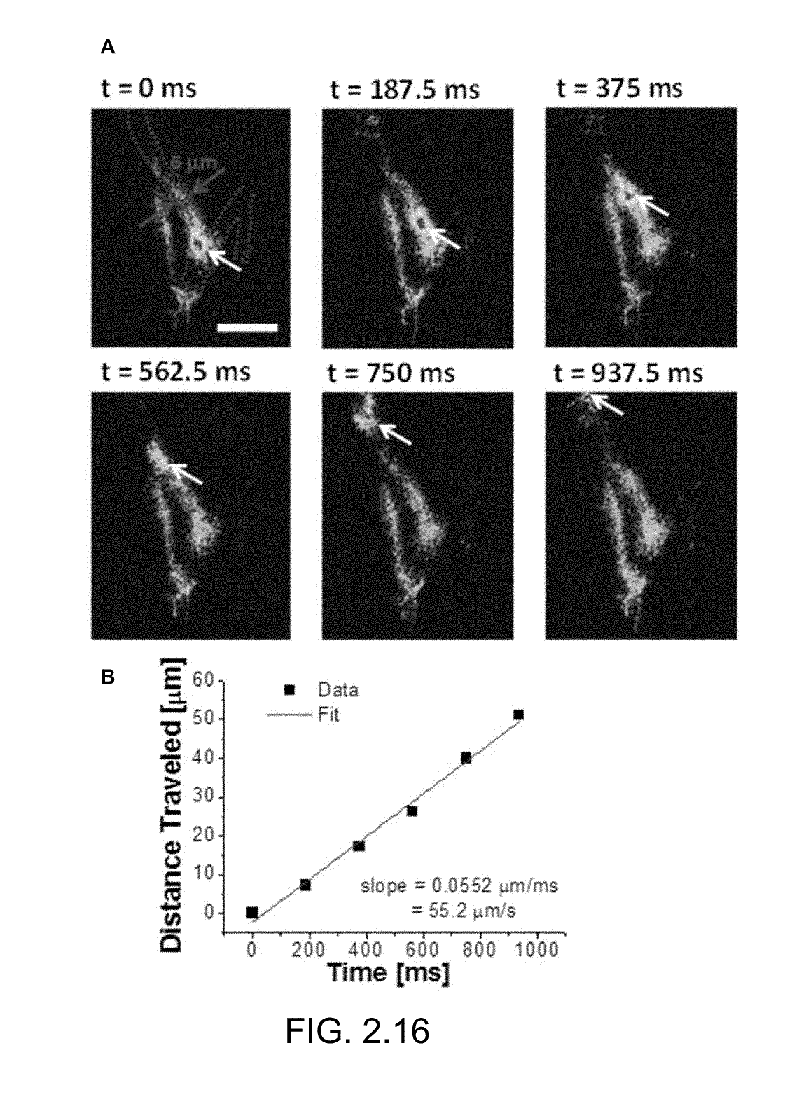

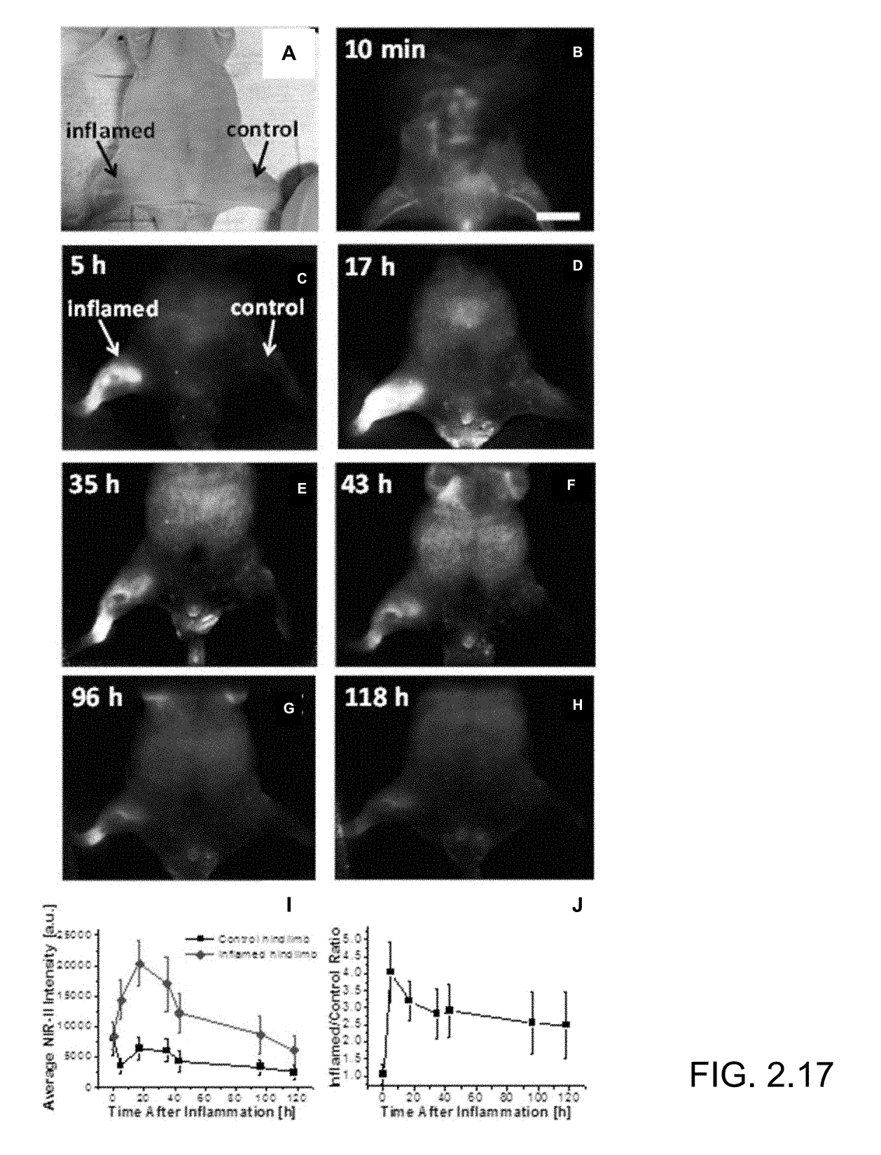

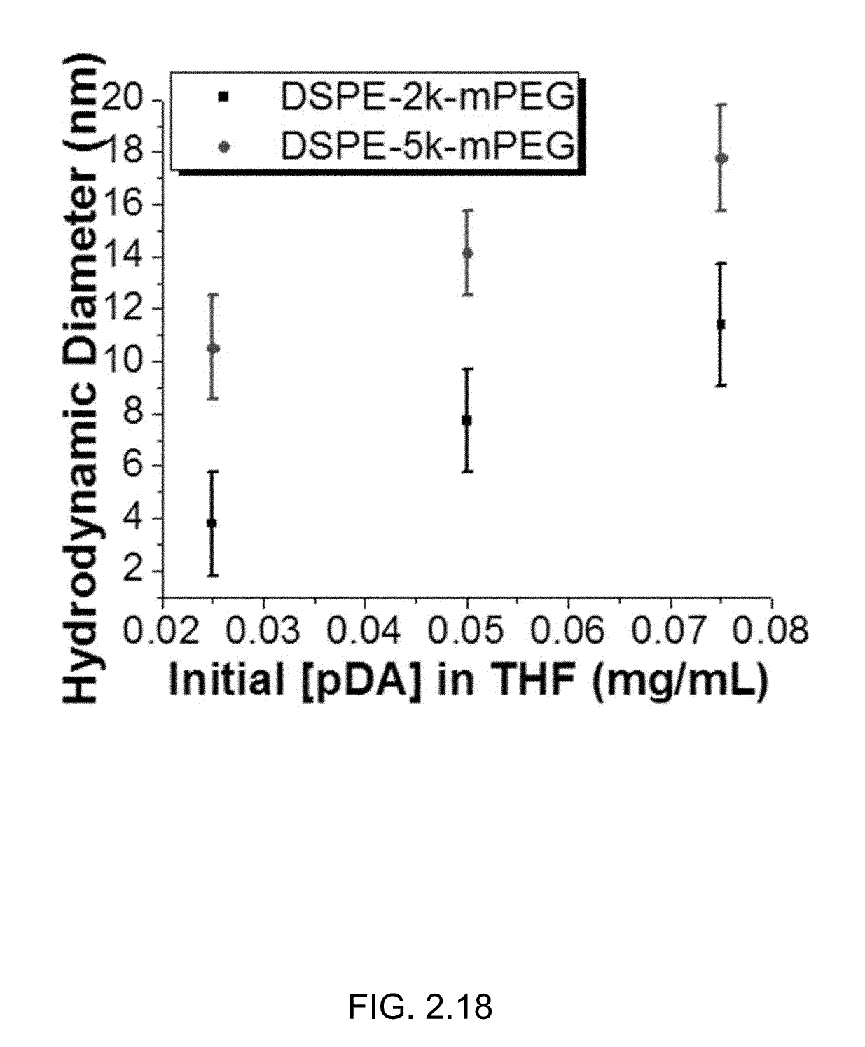

The pDA polymer was synthesized through a copolymerization reaction of two monomers, 1-(4,6-dibromo-3-fluorothieno[3,4-b]thiophen-2-yl)nonan-1-one and 2, 6-bis(trimethyltin)-4,8-bis (2-ethylhexyloxy)benzo[1,2-b; 3,4-b′]difuran.²⁶ Fluorine was introduced into the thienothiophene unit to tune the fluorescence to long wavelengths (FIG. 2.5). The pDA polymer was characterized by ¹H NMR spectroscopy (see Methods and Supplementary FIGS. 2.6-2.9) and gel permeation chromatography (GPC). The average molecular weight (Mₙ) of the polymer was ˜16 kDa (see Methods and FIG. 2.10). We dissolved the as-made pDA polymer in tetrahydrofuran (THF) and then mixed it with an aqueous solution of a biocompatible surfactant of PEGylated phospholipid [DSPE-mPEG (5 kDa)]^{1,3,19} (see Methods). The mixture was dialyzed against water to remove THF, resulting in a stable aqueous solution of supramolecular conjugate comprised of hydrophobic core of pDA and hydrophilic shell of DSPE-mPEG coating (FIG. 2.1A; named as pDA-PEG).

Atomic force microscopy (AFM) revealed the size distribution of pDA-PEG nanoparticles (FIG. 2.1B) with an average size of 2.9 nm when dried on a silicon surface (FIG. 2.11). The UV-Vis-NIR absorption spectrum of the as-made pDA-PEG solution in water exhibited an absorption peak at 654 nm, while the fluorescence emission spectrum showed a main emission peak at 1047 nm, suggesting a large Stokes shift of ˜400 nm (FIG. 2.1C). The pDA copolymer has been reported to have red-shifted absorption and emission compared to the corresponding homopolymers, due to the formation of a charge-transfer structure between the electron donor and acceptor units.^{26,27,30} The fluorescence quantum yield of pDA-PEG in a solution (FIG. 2.1D) under an excitation of 808 nm was ˜1.7%, measured against a standard IR-26 dye as a reference^{31,32} (FIG. 2.12A-F, where the excitation of 808 nm was intentionally chosen to balance absorption and scattering to afford maximum penetration depth of excitation light for in vivo imaging). The quantum yield was much higher than that of typical nanotubes (˜0.4%) (FIG. 2.12G-I). The pDA-PEG exhibited high photostability in phosphate buffer saline (PBS) and fetal bovine serum with negligible decay under continuous excitation for 1 h (FIG. 2.1E-F and FIG. 2.13). These results suggested pDA-PEG as an aqueous soluble, photo-stable and high brightness NIR-II fluorescent agent suited for biological imaging.

Molecular Imaging of Cells with pDA-PEG in NIR II.

We investigated pDA-PEG as a fluorescent label capable of targeting specific molecules on cell surfaces for performing molecular imaging of cancer cells through functionalization of pDA with targeting ligands. We made pDA-PEG-NH₂ using an amine-terminated phospholipid surfactant, DSPE-PEG-NH₂.^{33,34} Cetuximab (Erbitux) antibodies were then thiolated and conjugated to pDA-PEG-NH₂ via standard crosslinking reaction between the —NH₂ groups on the polymer and —SH groups on the thiolated antibody (see Methods). Then the pDA-PEG-Erbitux was applied to target the epidermal growth factor receptors (EGFR) on the cell membranes of EGFR-positive breast tumor MDA-MB-468 cells while the EGFR-negative brain tumor U87-MG cells were used as a negative control.¹⁵ Cell imaging in NIR-II detected>1000 nm fluorescence of pDA-PEG-Erbitux (FIG. 2.2) selectively on the EGFR-positive MDA-MB-468 cells and not on the negative U87-MG cells, showing a positive/negative ratio of ˜5.8. Thus, the pDA-PEG was made into an NIR-II fluorescent agent capable of recognizing and staining live cells with molecular specificity. This opened up the possibility of molecular imaging with conjugated copolymers in vitro and in vivo, which would not be attainable by structural imaging techniques such as ultrasound and optical coherence tomography (OCT).^{35,36}

Ultrafast Blood Flow Tracking with pDA PEG in NIR-II.

Next, we performed in vivo mouse blood vessel imaging by detecting the >1000 nm fluorescence of intravenously injected pDA-PEG solutions. Owing to the bright fluorescence of pDA-PEG with a 4-fold higher quantum yield (1.7%) than nanotube fluorophores³ (quantum yield ˜0.4%), we were able to dynamically image and track real-time arterial blood flow in the mouse hindlimb with a much shorter exposure time (˜20 ms) than previously possible (˜100 ms) in the NIR-II window.³ Immediately following a tail-vein injection of 200 μL solution of pDA-PEG, we performed video-rate imaging of the left hindlimb of a mouse in the supine position with an ultrafast frame rate of 25.6 frames per second (fps) compared to the previously achievable 5.3 fps.³ Such a high imaging speed enabled clear observation of the fast-moving flow front in the femoral artery as the NIR-II emitting pDA-PEG entered the hindlimb (FIG. 2.3A). The average femoral blood flow velocity was quantified by plotting the distance traveled by the flow front as a function of time, showing an overall linear increase with an average blood velocity of 4.36 cm·s⁻¹ (FIG. 2.3B), consistent with measurement by ultrasound (4˜7 cm·s⁻¹).³ This represented the first in vivo blood flow velocity measurement by direct tracking through real-space imaging of fast-moving flow front on the millisecond scale using NIR-II fluorescence.

Interestingly, high time-resolution analysis of blood flow-front position versus time revealed periodic variations in instantaneous velocity over time with a period of 150-200 ms, showing oscillations between the highest instantaneous blood velocity of ˜8 cm·s⁻¹ and the lowest blood velocity of ˜2 cm·s⁻¹, corresponding to the ventricular ejection (systolic) and ventricular relaxation (diastolic) phases respectively of a single cardiac cycle (FIG. 2.3C). The observation of such a rapid periodic oscillation in the femoral blood velocity was entirely owed to the high time resolution of 20 ms, much shorter than the cardiac cycle of ˜200 ms. The fast arterial blood flow (average speed ˜4.36 cm·s⁻¹) passed through the entire femoral artery (length ˜2 cm) within two cardiac cycles (˜400 ms). At longer times post injection, the same hindlimb imaged at 39 s post injection (p.i.) showed full perfusion of the injected NIR-II pDA-PEG fluorescent polymer into the femoral artery (FIG. 2.3D).

To further glean the oscillating blood flow front with high time-resolution, we selected a region of interest (ROI) of the femoral artery and analyzed the NIR-II fluorescence intensity as a function of time (FIG. 2.4A). We observed evenly spaced intensity humps over a linearly increasing baseline (FIG. 2.4B), with the humps corresponding to the systolic phases of cardiac cycles of the mouse and the increasing baseline due to increased overall perfusion of pDA-PEG fluorophore into the artery vessel.³ By subtracting the linear rise from the intensity vs. time curve, we observed clear consecutive intensity spikes in the time course due to periodic ventricular ejections (FIG. 2.4C). Further, the linear-baseline subtracted fluorescence intensity vs. time was made into a movie that clearly showed the blood flow variations over the two phases of each heartbeat (FIGS. 2.4D-J). The blood flow vs. time showed a similar cardiogram waveform (FIG. 2.4C) as previous Doppler ultrasound measurement,³⁷ demonstrating fluorescence imaging could help visualize rapid blood flow changes in real-space and real-time within a single cardiac cycle of mouse.

It is also interesting to note that the dots observed along the femoral artery in the baseline-subtracted images (FIGS. 2.4F-H) were at fixed positions over time and corresponded to local higher NIR-II intensity regions along the femoral artery. These high intensity local regions were found to coincide with locations along the vessel with slightly larger vessel diameters (by ˜20%) of the lumen measured with the vessel image. That is, more NIR-II pDA-PEG fluorophores in the blood were filled into a larger lumen and thus exhibited higher NIR-II intensity (FIG. 2.14). From the NIR-II fluorescence oscillations with an average period of 206.7 ms per cardiac cycle (FIG. 2.4K), we measured a heart rate of 290 beats per minute for the mouse, which agreed with previous results via cardiac gating.³⁸ Thus, the high-temporal and high-spatial resolution NIR-II imaging afforded by the high brightness of the conjugated polymer led to remarkably rich details of blood flows and cardiac cycles in vivo.

### Discussion:

In vivo fluorescence imaging of live animals in the NIR-II region benefits from deeper penetration of up to a few millimeters inside the body and reduced scattering of photons that scales inversely proportional with wavelength as ˜λ^{-w} (w=0.22−1.68) in biological tissues.^{3,9} Conjugated polymers have been widely used in organic solar cells,^{26,39} light-emitting diodes^{40,41} and organic electronics.^{42,43} Fluorescent imaging with conjugated polymers has been limited to emission wavelength <900 nm.⁴⁴⁻⁴⁶ Our current work developed the NIR-II agent with >1000 nm fluorescence based on conjugated polymers for biological imaging both in vitro and in vivo.⁴⁷ The facile synthetic route of donor-acceptor copolymers could allow further tuning of the optical properties of the polymers through modifying the donor-acceptor structures (FIG. 2.15) and the length of the copolymer. A more electron-donating donor and a more electron-withdrawing acceptor typically result in smaller band gap of the copolymer,⁴⁸ and a longer copolymer exhibits reduced band gap due to a greater delocalization of π-electrons.³⁰ This could lead to a library of polymers with tunable excitation and emission wavelengths for NIR-II imaging (see FIG. 2.15 for several polymers synthesized thus far). With such development, we envisage fluorophores with different emission wavelengths in the 1.0-1.7 μm NIR-II window, allowing for multicolor molecular imaging of different biomarkers. Also, further improved penetration depth could be achieved in vivo by tuning the emission wavelength.

A salient advantage of the pDA-PEG polymer as an NIR-II fluorophore for in vivo live animal imaging is its much higher quantum yield (˜1.7%) than SWNTs (˜0.4%) previously used for hindlimb blood flow tracking.³ This allows for much faster (20 ms exposure time vs. previous 100 ms)³ video-rate imaging of dynamic changes in the blood flow labeled by NIR-II fluorophore, pushing the limit of temporal resolution to a previously unattainable level of >25 fps (the instrument frame rate limit is 50 fps due to an overhead time of 19 ms). Although the pDA-PEG polymer has similar quantum efficiency (˜1.7%) to nanoparticles of small organic molecules (˜1.8%),¹⁸ since the major peak of the small organic molecule nanoparticles is located at ˜920 nm while the major peak of pDA-PEG polymer is at 1050 nm, the pDA-PEG polymer has a greater portion of emitted photons in the >1000 nm NIR-II region (˜70%) than the small molecule nanoparticles (˜40%).¹⁸ Taken together, the high fluorescence quantum yield, red-shifted fluorescence emission in the NIR-II region and low density of the pDA polymer require 10˜100× lower mass dose of injection than previous NIR-II fluorophores (such as SWNTs^{1-3,9-12}, QDs^{6,7,15-17}, rare-earth doped nanoparticles⁸ and nanoparticles incorporating organic molecules¹⁸) to reach the same in vivo imaging quality, and allow for dynamic fluorescence imaging with much shorter exposure time and higher temporal resolution. This high temporal resolution well exceeds the heart rate of mice (˜5 beats per second, or ˜200 ms per cycle) by 5 times, eliminating the need for cardiac gating typically required for low-frame-rate cardiovascular imaging techniques.^{38,49}

Previous fluorescence imaging and tracking of blood flow usually require the removal of scattering tissue over the vessels of interest to afford higher spatial resolution, and gating devices to eliminate image blurring due to cardiac and respiratory motions on a faster time scale than the temporal resolution of image acquisitions (˜10 fps).^{38,50} Ultrasound imaging and optical coherence tomography (OCT) with ultrafast image acquisition rate (kHz˜MHz) have shown the advantages of measuring fast blood flow and cardiac cycles,⁵¹⁻⁵³ but the spatial resolution (in the 10 μm˜1 mm range) and signal-to-noise ratio are sub-optimal due to long wavelengths⁵⁴ and speckles.^{55,56} Here, by utilizing NIR-II fluorescence with reduced tissue scattering and an ultrafast frame rate of >25 fps, we have enabled simultaneous blood velocity and cardiac cycle measurements as well as high resolution imaging of blood vessels 1-2 mm deep under otherwise highly scattering skin tissues.⁵⁷ We have also proved the concept that the polymer-based NIR-II fluorophores can be used to track blood flow in capillaries with sub-10 μm diameters (FIG. 2.16A), which are well below the spatial resolutions of traditional ultrasound and OCT. Based on the capillary blood flow tracking images, we measured the capillary blood velocity to be ˜55.2 μm·s⁻¹ (FIG. 2.16B), in good agreement with previous studies.^{58,59} The dynamic range of blood velocity measurement in our NIR-II imaging system using the pDA fluorophores is derived as 0 to 640 mm/s based on the brightness of the fluorophore and the detector sensitivity. In addition, we have shown that the pDA-PEG fluorophores can be used to track regional blood flow and redistribution as a result of increased metabolic demand after heat-induced inflammation of the tissue (FIG. 2.17), providing a direct diagnostic tool for visualizing the metabolic difference of the tissue.

The Dynamic Range of Blood Velocity Measurement with the NIR-II Fluorescence of pDA-PEG.

The dynamic range of velocity measurement using the pDA-PEG fluorophores is from 0 to ˜640 mm/s, determined by both the optical properties (the NIR-II fluorescence brightness) of pDA-PEG and the speed of data acquisition of the camera used for dynamic imaging. The detailed derivation of the dynamic range is shown as follows.

Since velocity is defined as distance/time, we need to find out the dynamic ranges of available time intervals of image acquisition and the measurable distance in our imaging system. The frame rate of dynamic fluorescence imaging (i.e., the inverse of time interval per image acquisition), which determines how fast we can track the blood flow in vivo, is given by 1/(exposure time+overhead time). Here the exposure time needed for acquiring images with good quality is determined by the brightness of the pDA fluorophores, and the overhead time is determined by the rate at which the camera acquires and digitizes each image. The shortest possible exposure time is 20 ms (using the brightest pDA polymer we currently have), while the shortest possible overhead time of the camera is ˜19 ms. Therefore, the maximum frame rate we can use for dynamic imaging is 1/(20 ms+19 ms)=25.6 Hz (or 25.6 fps). Our imaging system is a low-pass system, meaning any frequency lower than 25.6 Hz is available by increasing the time interval between measurements. Therefore the dynamic range of temporal resolution is from 0 to 25.6 Hz.

On the other hand, by using different lens/objective sets to form images with different magnifications onto the camera, the measurable distance during dynamic imaging ranges from 1 □m (diffraction limit) to 25 mm (field of view limit). Since velocity is distance divided by time, one can derive the slowest and the fastest measurable blood velocity in our current setup using the pDA fluorophores as follows:

The slowest measurable blood velocity=1μm×0 Hz=0 mm/s

The fastest measurable blood velocity=25 mm×25.6 Hz=640 mm/s

Therefore the dynamic range of measurable blood velocity in our current system using the pDA fluorophores is given as 0 to 640 mm/s.

The heavy-metal-free nature of fluorescent conjugated polymers bodes well for potentially low toxicity agents for in vivo applications.⁶⁰ In a cellular toxicity study, we found that the toxicity of pDA-PEG nanoparticles depended on the surfactant coating, and a surfactant with branched PEG exhibited alleviated toxicity (see Methods). The mice injected with pDA-PEG were monitored over a period of up to 2 months without showing obvious toxic effects or health problems. We also attempted tuning the size of the pDA-PEG nanoparticles and obtained the smallest pDA-PEG with an average hydrodynamic size of <6 nm (FIG. 2.18, and also see Methods for more information). Systematic investigation is required to study the long term fate and toxicology of pDA-PEG nanoparticles, which is beyond the scope of the current work.

Real-time hemodynamic imaging could be of central importance to better understanding various cardiovascular diseases and designing treatment strategies.⁶¹ An ideal NIR-II fluorescent agent capable of achieving hemodynamic imaging with high spatial and temporal resolutions should have tunable emission wavelength in the >1000 nm region to minimize tissue scattering, high fluorescence quantum efficiency (>5%) for ultra-short imaging exposure time. The current work opens up future research to achieve these goals, and could eventually lead to NIR-II fluorescence agents suitable for clinic use.

### Methods:

Synthesis of pDA Copolymer.

The synthesis of monomers M1 and M2 can be found in previous publications.^{62,64} For the synthesis of the pDA copolymer, 0.091 g of monomer M1 (0.2 mmol), 0.148 g of the monomer M2 (0.2 mmol) and 15 mL of anhydrous toluene were mixed in a two-neck flask. The solution was flushed with N₂ for 10 min, and 15 mg of Pd(PPh₃)₄ was added into the flask. Then the solution was flushed with N₂ for another 25 min. The two-neck flask was placed in an oil bath and heated up carefully to 110° C. The mixture was stirred for 24 h at 110° C. under N₂ atmosphere. After the reaction was complete, the mixture was allowed to cool down to room temperature, and the polymer was precipitated by adding 100 mL of methanol and filtered through a Soxhlet thimble. Soxhlet extraction was performed with methanol, hexanes and chloroform. 80 mg of the polymer was obtained as a green solid from the chloroform fraction by rotary evaporation, and dried under vacuum overnight with a moderate yield of 56%.

Characterizations of Monomers and pDA Copolymer.

All compounds, including the monomers M1 and M2, and the pDA copolymer, were characterized by nuclear magnetic resonance (NMR) spectroscopy. The ¹H NMR and ¹⁹F NMR spectra were measured on a Bruker AV-400 spectrometer at room temperature. Chemical shifts were described as δ values (ppm), where tetramethylsilane (TMS) was used as an internal reference. Splitting patterns were labeled as s (singlet), d (doublet), t (triplet), m (multiplet) and br (broad). As such, the NMR spectral assignments for the two monomers M1 and M2, as well as the product polymer, are given as follows,

### Monomer M1:

¹H NMR (400 MHz, CDCl₃): δ 2.95 (t, 2H), 1.76 (m, 2H), 1.39-1.30 (m, 10H), 0.91 (t, 3H).

¹⁹F NMR (400 MHz, CDCl₃): −129 ppm (s, Ar-F)

### Monomer M2:

¹H NMR (400 MHz, CDCl₃): δ 7.06 (s, 2H), 4.35 (d, 4H), 1.78 (m, 2H), 1.37-1.70 (m, 16H), 0.96-1.03 (m, 12H), 0.44 (s, 18H).

### Polymer pDA:

¹H NMR (400 MHz, CDCl₃): δ 6.80 (br, 2H), 4.31 (br, 4H), 3.06 (br, 2H), 2.01-1.21 (br, 30H), 0.81-1.21 (br, 15H).

Elemental analysis of the pDA copolymer was performed on a Flash EA 1112 elemental analyzer. Elemental composition of pDA copolymer was calculated based on the molecular formula (C₄₁H₅₃FO₅S₂)n as C: 69.26%; H: 7.80%; O: 11.25%, and compared to measured elemental composition by the elemental analyzer: C, 70.08%; H, 7.73%; O, 11.09%.

Molecular weight and polydispersity of the pDA copolymer were measured by gel permeation chromatography (GPC). The number-average molecular weights (Mₙ), weight-average molecular weights (M_{w}) and polydispersity index (PDI, M_{w}/Mₙ) were determined using polystyrene as the reference. Measurements were carried out using a Waters 515 HPLC pump, a Waters 2414 differential refractometer, and three Waters Styragel columns (HT2, HT3 and HT4) with THF as the eluent at a flow rate of 1.0 mL·min⁻¹ at a temperature of 35° C.

Preparation of pDA-PEG and pDA-PEG-NH2.

The as-synthesized pDA polymer was dissolved in THF at a mass concentration of 75 μg·mL⁻¹ and mixed with an aqueous solution of DSPE-mPEG (5 kDa) (1,2-distearoyl-sn-glycero-3-phosphoethanolamine-N-[methoxy(polyethyleneglycol, 5000)], Laysan Bio) at a concentration of 1.1 mg·mL⁻¹ with a 1:9 volume ratio for making the pDA-PEG, or with an aqueous solution of DSPE-PEG-NH2 (5 kDa) (1,2-distearoyl-sn-glycero-3-phosphoethanolamine-N-[amino(polyethyleneglycol, 5000)], Laysan Bio) at 1.1 mg. mL⁻¹ with a 1:9 volume ratio for making the pDA-PEG-NH2. The pDA polymer remained soluble in the mixed solvent without any precipitation. Then the mixture was dialyzed against water to remove THF and make a THF-free, clear aqueous solution of pDA-PEG or pDA-PEG-NH2 polymers. To remove aggregates formed during dialysis, the suspension was ultracentrifuged for 30 min at 300,000 g and only the supernatant was retained. Free, unbound surfactant in the solution was removed through 30-kDa centrifugal filters (Amicon) without causing any instability to the pDA-PEG polymers, suggesting the hydrophobic core of pDA and the hydrophilic shell of DSPE-mPEG were making a strongly-held complex.

Preparation of pDA-PEG-Erbitux Bioconjugate.

A 200 μL solution of pDA-PEG-NH2 after excess surfactant removal was concentrated to a mass concentration of 320 μg·mL⁻¹, determined by a mass extinction coefficient of 30.4 L·g⁻¹·cm⁻¹ at its peak absorbance in the UV-Vis-NIR absorption spectrum. Then this solution was mixed with 1 mM sulfo-SMCC for 1 h in PBS at pH 7.4. On the other hand, 12.3 μL of Erbitux solution (Bristol-Myers Squibb) at a concentration of 2 mg·mL⁻¹ (˜13.3 μm) was mixed with a 1.64 μL solution of Traut's reagent (Sigma) at 1 mM concentration and 86 μL PBS solution. In the mixture the molar ratio of Erbitux antibody to Traut's reagent was 1:10. The mixture of Erbitux antibody and Traut's reagent was allowed to react for 1.5 h at room temperature. After removing excess sulfo-SMCC from the polymer solution and excess Traut's reagent from the antibody solution by filtration through 30-kDa centrifugal filters (Amicon), the two solutions were mixed and allowed to react for 2 days in PBS at 4° C. to make the pDA-PEG-Erbitux bioconjugate.

Spectral Charaterizations of pDA-PEG.

UV-Vis-NIR absorption spectrum of pDA-PEG polymer in water was measured by a Cary 6000i UV-Vis-NIR spectrophotometer, background-corrected for contribution from water. The measured range was 400-900 nm. The NIR-II fluorescence spectrum was taken using a home-built NIR-II spectroscopy setup in the 900-1500 nm region. The excitation source was an 808-nm diode laser (RMPC lasers) at an output power of 160 mW and filtered by an 850-nm short-pass filter (Thorlabs), a 1000-nm short-pass filter (Thorlabs), an 1100-nm short-pass filter (Omega) and a 1300-nm short-pass filter (Omega). The excitation at 808 nm was intentionally chosen to balance absorption and scattering to afford maximum penetration depth of excitation light for in vivo imaging. The excitation beam was allowed to pass through an aqueous solution of pDA-PEG polymer in a 1 mm path cuvette (Starna Cells) and the emission was collected in the transmission geometry with a 900-nm long-pass filter (Thorlabs) to reject the excitation light. The emitted fluorescence from the sample was directed into a spectrometer (Acton SP2300i) equipped with a liquid-nitrogen-cooled InGaAs linear array detector (Princeton OMA-V). The emission spectrum was corrected after raw data acquisition to account for the detector sensitivity profile and extinction feature of the filter using the MATLAB software.

Determination of Fluorescence Quantum Yield of pDA-PEG.

Fluorescence quantum yield of the pDA-PEG polymer in water was measured in a similar way to a previous publication,³² using the NIR-II fluorescent IR-26 dye as the reference (quantum yield=0.5%).³¹ For reference calibration on our setup, a stock solution of 1 mg·mL⁻¹ IR-26 dissolved in 1,2-dichloroethane (DCE) was diluted to a DCE solution with its absorbance value of ˜0.10 at 808 nm. Then this sample was diluted using DCE to a series of samples with their absorbance values at 808 nm of ˜0.08, ˜0.06, ˜0.04 and ˜0.02. Using these highly diluted samples with low absorbance minimized any secondary optical processes such as reabsorption and reemission effects. Then a total of five IR-26 solutions in DCE with linearly spaced concentrations (including the one with absorbance of ˜0.10) were loaded into a 10-mm path fluorescence cuvette (Starna Cells) one at a time. The excitation source was an 808-nm diode laser (RMPC lasers) with a linewidth FWHM of 1.89 nm at an output power of 160 mW and filtered by an 850-nm short-pass filter (Thorlabs), a 1000-nm short-pass filter (Thorlabs), an 1100-nm short-pass filter (Omega) and a 1300-nm short-pass filter (Omega). The emission was collected in the transmission geometry with a 900-nm long-pass filter (Thorlabs) to reject the excitation light and the emission spectrum was taken in the 900-1500 nm region. The same absorption and emission measurements were carried out for pDA-PEG polymers and SWNTs in aqueous solutions too. Then all emission spectra of both the reference and the samples were corrected after raw data acquisition to account for the detector sensitivity profile and the extinction profiles of the filters, and integrated in the 900-1500 nm NIR-II region. The definition of quantum yield dictates that all emitted photons should be taken into account for quantum yield measurement.′ In our quantum yield measurements, since an 808-nm laser is used, ideally the integration of the emission spectra should start from 809 nm to cover the complete emission band; however, due to the limited choice of excitation and emission filters, we started the integration from 900 nm, which would give an underestimated quantum yield. Nonetheless, this underestimation would not be too much since one could see from the emission spectra that the emission dropped rapidly below 950 nm, meaning there would not be too much fluorescence emission under 900 nm. The integrated NIR-II fluorescence intensity was plotted against absorbance at the excitation wavelength of 808 nm and fitted into a linear function. Two slopes, one obtained from the reference of IR-26 in DCE and the other from the sample (pDA-PEG or SWNT), were employed in the calculation of the quantum yield of the sample, based on equation (1) as follows,

$${QY}_{sample} = {QY}_{ref} \cdot \frac{{slope}_{sample}}{{slope}_{ref}} \cdot {( \frac{{n}_{sample}}{{n}_{ref}} )}^{2} \qquad ( 1 )$$

where nₛₐₘₚₗₑ and n_{ref} are the refractive indices of water and DCE, respectively.

AFM imaging of pDA-PEG. AFM image of pDA-PEG was acquired with a Nanoscope IIIa multimode AFM in the tapping mode. The sample for imaging was prepared by drop-drying a highly diluted aqueous solution of pDA-PEG polymer at a mass concentration of 750 ng·L⁻¹ onto the SiO₂/silicon substrate without any post-processing steps. To plot the size distribution of the pDA-PEG polymeric nanoparticles, 100 nanoparticles were measured from the AFM images and their heights were used to generate the histogram of size distribution. Due to the effect of tip size convolution in AFM,⁶⁶ the height measurement from the AFM micrograph, rather than the lateral size measurement, was used to measure the size of the nanoparticles deposited on the substrate.

Dynamic Light Scattering (DLS) Analysis of pDA-PEG.

DLS was performed using a solution of pDA-PEG at 0.75 μg·ml⁻¹ in PBS in a 1-cm quartz cuvette. A PBS solution was used to mimic the ionic strength in blood because the hydrodynamic size of pDA-PEG was dependent on the ionic strength. The data was collected using a Brookhaven Instruments 90Plus Particle Size Analyzer.

Cell Incubation and Staining.

All cell culture media were supplemented with 10% fetal bovine serum, 100 μg·mL⁻¹ streptomycin, 100 IU·mL⁻¹ penicillin and L-glutamine. The human glioblastoma U87-MG cells were cultured in Low Glucose Dulbecco's Modified Eagle Medium (DMEM), with 1 g·L⁻¹D-glucose and 110 mg·L⁻¹ sodium pyruvate, in the presence of 5% CO₂. Human breast cancer MDA-MB-468 cells were cultured in Leibovitz's L-15 medium at 37° C., CO₂ free. Cells were maintained in a 37° C. incubator. For cell staining, cells were trypsinized before the as-made pDA-PEG-Erbitux bioconjugate was incubated with both EGFR-positive (MDA-MB-468) and EGFR-negative (U87-MG) cell lines at a concentration of 5 μg·mL⁻¹ at 4° C. for 1 h, followed by washing the cells with 1×PBS to remove all free, unbound pDA-PEG-Erbitux bioconjugate in the suspension.

NIR-II Fluorescence Imaging of Cancer Cells.

Targeted cell imaging in the 1.0-1.7 μm NIR-II window was carried out using a 658-nm laser diode with a 150 μm-diameter excitation beam focused by a ×50 objective lens (Olympus). The NIR-II fluorescence from pDA-PEG-Erbitux bioconjugate was collected using a liquid-nitrogen-cooled, 320×256 pixel, 2D InGaAs camera (Princeton Instruments 2D OMA-V). The excitation light was filtered out using a 900 nm and a 1000 nm long-pass filter (Thorlabs) so that the intensity of each pixel in the camera represented fluorescence in the 1.0-1.7 μm NIR-II region. The exposure time was 3 s for all fluorescence images.

Mouse Handling.

All vertebrate animal experiments were performed under the approval of Stanford University's Administrative Panel on Laboratory Animal Care (APLAC). 8-week old female BALB/c mice were obtained from Charles River and housed at the Research Animal Facility of Stanford under our approved animal protocols. Before hindlimb vessel imaging, all mice were anesthetized in a rodent anesthesia machine with 2 L·min⁻¹ 02 gas flow mixed with 3% Isoflurane. Then the hair over the hindlimb skin was carefully removed using Nair to avoid causing wounds to the skin. Tail vein injection of the pDA-PEG contrast agent was carried out in dark and synchronized with the camera that started continuous image acquisition simultaneously. The injected dose was a 200 μL bolus of the pDA-PEG in a 1×PBS solution at a mass concentration of 0.25 mg·mL⁻¹. During the time course of imaging the mouse was kept anesthetized by a nose cone delivering 1.5 L·min⁻¹ O₂ gas mixed with 3% Isoflurane.

Ultrafast Video-Rate NIR II Vessel Imaging.

Video-rate NIR-II vessel imaging was done in a similar manner as our previous publication.³ In brief, imaging was carried out on a homebuilt imaging setup consisting of a 2D InGaAs camera (Princeton Instruments 2D OMA-V). The excitation was provided by an optical-fiber-coupled 808-nm diode laser (RMPC Lasers), intentionally chosen to balance absorption and scattering to afford maximum penetration depth of excitation light for in vivo imaging. The high fluorescence quantum yield of the pDA-PEG also ensured sufficient emission for ultra-fast in vivo imaging despite the non-resonant excitation under 808 nm. The light was collimated by a collimator with a focal length of 4.5 mm (ThorLabs) and filtered by an 850-nm and a 1000-nm shortpass filter (ThorLabs). The power density of the excitation laser at the imaging plane was 140 mW·cm⁻², significantly lower than the reported safe exposure limit of 329 mW·cm⁻² at 808 nm.⁶⁷ The emitted fluorescence from the mouse was allowed to pass through a 900 nm and a 1000 nm long-pass filter (Thorlabs) and to be focused onto the InGaAs detector by a lens pair consisting of two NIR achromats (200 and 75 mm; Thorlabs). The distance between the two NIR achromats was adjusted to have only one hindlimb of the mouse included in the field of view. The camera was set to expose continuously using the LabVIEW software with an exposure time of 20 ms. NIR-II images were acquired with a frame rate of 25.6 fps due to a 19 ms overhead time during readout.

In Vitro Toxicity Study of pDA-PEG.

We determined the in vitro toxicity of pDA-PEG by MTS assay using a CellTiter96 kit (Promega) on human breast cancer MDA-MB-468 cells. To evaluate how the surfactant coating of the pDA polymer affected the nanoparticles' in vitro toxicity, we coated the pDA polymer (the same formulation as in FIG. 2.5) with DSPE-mPEG and poly(maleic anhydride-alt-1-octadecene)methoxy(polyethyleneglycol, 5 kDa) (C18-PMH-mPEG), respectively, where DSPE-mPEG has one linear PEG chain while C18-PMH-mPEG has many branched PEG chains. For in vitro toxicity study, approximately 5,000 MDA-MB-468 cells were incubated per well with 100 μl of Leibovitz's L-15 medium spiked with pDA-DSPE-mPEG or pDA-C18-PMH-mPEG at serially diluted concentrations (n=6 for each concentration). The cells were kept at 37° C. in a humidified atmosphere for 36 h in the absence of CO₂. 100% MDA-MB-468 cell medium without any pDA-PEG was taken as the positive control while MDA-MB-468 cell medium spiked with 20 μM Doxil® was taken as the negative control. Immediately before addition of 15 μl of CellTiter96 for the colorimetric assay, the polymer-spiked medium was removed from each well in the plate and replaced with fresh MDA-MB-468 cell growth medium. This would prevent any interference in the absorbance readings from the intrinsic color of pDA-PEG. After 1.5 h, the color change was quantified using a plate reader, which took absorbance readings at 485 nm. Cell viability was plotted as a fraction of the absorbance value of the positive control wells incubated without any pDA-PEG after baseline subtraction of the absorbance for medium alone. The negative control wells had an average cell viability value of ˜60% after 36 h of incubation, which was as expected. We found from the in vitro toxicity study that pDA-DSPE-mPEG has a half maximal inhibitory concentration (IC50) level of ˜30 μg/mL, while pDA-C18-PMH-mPEG has an IC50 level of >150 μg/mL. The pDA-PEG solution could not be further concentrated to an even higher concentration, preventing us from measuring the cell viability under an incubation concentration of >150 μg/mL. It is also noteworthy that the linear or branched PEG coating did not alter the fluorescence quantum efficiency to a noticeable level. Hydrodynamic size tuning of pDA-PEG nanoparticles. The hydrodynamic size tuning was performed by coating the pDA polymer with surfactants of different PEG molecular weights (2 kDa and 5 kDa) and changing the initial pDA concentration in the THF solution (0.025-0.075 mg/mL; please see the “Preparation of pDA-PEG and pDA-PEG-NH2” section in Methods) before mixing with 1.1 mg/mL DSPE-mPEG in water. After the pDA-PEG nanoparticles were made in each synthesis condition, dynamic light scattering (DLS) analysis was performed to evaluate each sample's hydrodynamic diameter as a function of initial pDA concentration in the THF solution and the molecular weight of PEG in the surfactant (please see the “Dynamic light scattering (DLS) analysis of pDA-PEG” section in Methods). The dependence of pDA-PEG hydrodynamic size on initial pDA concentration and PEG molecular weight is plotted in FIG. 2.18, showing that both smaller PEG and lower initial concentration of pDA favor the formation of smaller pDA-PEG nanoparticles in aqueous solution. However, it is noteworthy that DLS was not able to discriminate the ‘empty’ nanoparticles without any pDA molecules loaded inside, and therefore the size distribution of the measured hydrodynamic diameters below ˜4 nm could be due to the micelles formed by the DSPE-mPEG surfactants only.

### REFERENCES

1 Welsher, K., Liu, Z., Sherlock, S. P., Robinson, J. T., Chen, Z., Daranciang, D. & Dai, H. J. A route to brightly fluorescent carbon nanotubes for near-infrared imaging in mice. Nat. Nanotechnol. 4, 773-780 (2009).
2 Hong, G. S., Wu, J. Z., Robinson, J. T., Wang, H. L., Zhang, B. & Dai, H. J. Three-dimensional imaging of single nanotube molecule endocytosis on plasmonic substrates. Nat. Commun. 3, 700 (2012).
3 Hong, G. S., Lee, J. C., Robinson, J. T., Raaz, U., Xie, L. M., Huang, N. F., Cooke, J. P. & Dai, H. J. Multifunctional in vivo vascular imaging using near-infrared II fluorescence. Nat. Med. 18, 1841-1846 (2012).
4 Won, N., Jeong, S., Kim, K., Kwag, J., Park, J., Kim, S. G. & Kim, S. Imaging depths of near-infrared quantum dots in first and second optical windows. Mol. Imaging 11, 338-352 (2012).
5 Yi, H. J., Ghosh, D., Ham, M. H., Qi, J. F., Barone, P. W., Strano, M. S. & Belcher, A. M. M13 Phage-Functionalized Single-Walled Carbon Nanotubes As Nanoprobes for Second Near-Infrared Window Fluorescence Imaging of Targeted Tumors. Nano Lett. 12, 1176-1183 (2012).
6 Hong, G. S., Robinson, J. T., Zhang, Y. J., Diao, S., Antaris, A. L., Wang, Q. B. & Dai, H. J. In Vivo Fluorescence Imaging with Ag2S Quantum Dots in the Second Near-Infrared Region. Angew. Chem. Int. Ed. 51, 9818-9821 (2012).
7 Dong, B. H., Li, C. Y., Chen, G. C., Zhang, Y. J., Zhang, Y., Deng, M. J. & Wang, Q. B. Facile Synthesis of Highly Photoluminescent Ag2Se Quantum Dots as a New Fluorescent Probe in the Second Near-Infrared Window for in Vivo Imaging. Chem. Mater. 25, 2503-2509 (2013).
8 Naczynski, D. J., Tan, M. C., Zevon, M., Wall, B., Kohl, J., Kulesa, A., Chen, S., Roth, C. M., Riman, R. E. & Moghe, P. V. Rare-earth-doped biological composites as in vivo shortwave infrared reporters. Nat. Commun. 4, 2199 (2013).
9 Welsher, K., Sherlock, S. P. & Dai, H. J. Deep-tissue anatomical imaging of mice using carbon nanotube fluorophores in the second near-infrared window. P. Natl. Acad. Sci. USA 108, 8943-8948 (2011).
10 Robinson, J. T., Welsher, K., Tabakman, S. M., Sherlock, S. P., Wang, H. L., Luong, R. & Dai, H. J. High Performance In Vivo Near-IR (>1 mm) Imaging and Photothermal Cancer Therapy with Carbon Nanotubes. Nano Res. 3, 779-793 (2010).
11 Robinson, J. T., Hong, G. S., Liang, Y. Y., Zhang, B., Yaghi, 0. K. & Dai, H. J. In Vivo Fluorescence Imaging in the Second Near-Infrared Window with Long Circulating Carbon Nanotubes Capable of Ultrahigh Tumor Uptake. J. Am. Chem. Soc. 134, 10664-10669 (2012).
12 Diao, S., Hong, G. S., Robinson, J. T., Jiao, L. Y., Antaris, A. L., Wu, J. Z., Choi, C. L. & Dai, H. J. Chirality Enriched (12,1) and (11,3) Single-Walled Carbon Nanotubes for Biological Imaging. J. Am. Chem. Soc. 134, 16971-16974 (2012).
13 Liu, Z., Tabakman, S., Sherlock, S., Li, X. L., Chen, Z., Jiang, K. L., Fan, S. S. & Dai, H. J. Multiplexed Five-Color Molecular Imaging of Cancer Cells and Tumor Tissues with Carbon Nanotube Raman Tags in the Near-Infrared. Nano Res. 3, 222-233 (2010).
14 Liu, Z., Tabakman, S., Welsher, K. & Dai, H. J. Carbon Nanotubes in Biology and Medicine: In vitro and in vivo Detection, Imaging and Drug Delivery. Nano Res. 2, 85-120 (2009).
15 Zhang, Y., Hong, G. S., Zhang, Y. J., Chen, G. C., Li, F., Dai, H. J. & Wang, Q. B. Ag2S Quantum Dot: A Bright and Biocompatible Fluorescent Nanoprobe in the Second Near-Infrared Window. ACS Nano 6, 3695-3702 (2012).
16 Zhang, Y., Zhang, Y. J., Hong, G. S., He, W., Zhou, K., Yang, K., Li, F., Chen, G. C.,

Liu, Z., Dai, H. J. & Wang, Q. B. Biodistribution, pharmacokinetics and toxicology of Ag2S near-infrared quantum dots in mice. Biomaterials 34, 3639-3646 (2013).
17 Zhu, C. N., Jiang, P., Zhang, Z. L., Zhu, D. L., Tian, Z. Q. & Pang, D. W. Ag2Se Quantum Dots with Tunable Emission in the Second Near-Infrared Window. ACS Appl. Mater. Inter. 5, 1186-1189 (2013).
18 Tao, Z. M., Hong, G. S., Shinji, C., Chen, C. X., Diao, S., Antaris, A. L., Zhang, B., Zou, Y. P. & Dai, H. J. Biological Imaging Using Nanoparticles of Small Organic Molecules with Fluorescence Emission at Wavelengths Longer than 1,000 nm. Angew. Chem. Int. Ed. 52, 13002-13006 (2013).
19 Liu, Z., Davis, C., Cai, W. B., He, L., Chen, X. Y. & Dai, H. J. Circulation and long-term fate of functionalized, biocompatible single-walled carbon nanotubes in mice probed by Raman spectroscopy. P. Natl. Acad. Sci. USA 105, 1410-1415 (2008).
20 Schipper, M. L., Nakayama-Ratchford, N., Davis, C. R., Kam, N. W. S., Chu, P., Liu, Z., Sun, X. M., Dai, H. J. & Gambhir, S. S. A pilot toxicology study of single-walled carbon nanotubes in a small sample of mice. Nat. Nanotechnol. 3, 216-221 (2008).
21 O'Connell, M. J., Bachilo, S. M., Huffman, C. B., Moore, V. C., Strano, M. S., Haroz, E. H., Rialon, K. L., Boul, P. J., Noon, W. H., Kittrell, C., Ma, J. P., Hauge, R. H., Weisman, R. B. & Smalley, R. E. Band gap fluorescence from individual single-walled carbon nanotubes. Science 297, 593-596 (2002).
22 Zhang, Q. T. & Tour, J. M. Low optical bandgap polythiophenes by an alternating donor/acceptor repeat unit strategy. J. Am. Chem. Soc. 119, 5065-5066 (1997).
23 Greenham, N. C., Moratti, S. C., Bradley, D. D. C., Friend, R. H. & Holmes, A. B. Efficient Light-Emitting-Diodes Based on Polymers with High Electron-Affinities. Nature 365, 628-630 (1993).
24 Zhang, Z. G., Min, J., Zhang, S. Y., Zhang, J., Zhang, M. J. & Li, Y. F. Alkyl chain engineering on a dithieno[3,2-b:2′,3′-d]silole-alt-dithienylthiazolo[5,4-d]thiazole copolymer toward high performance bulk heterojunction solar cells. Chem. Commun. 47, 9474-9476 (2011).
25 Cui, C. H., Fan, X., Zhang, M. J., Zhang, J., Min, J. & Li, Y. F. A D-A copolymer of dithienosilole and a new acceptor unit of naphtho[2,3-c]thiophene-4,9-dione for efficient polymer solar cells. Chem. Commun. 47, 11345-11347 (2011).
26 Liu, B., Chen, X. W., Zou, Y. P., He, Y. H., Xiao, L., Xu, X. J., Li, L. D. & Li, Y. F. A benzo[1,2-b: 4,5-b′]difuran- and thieno-[3,4-b]thiophene-based low bandgap copolymer for photovoltaic applications. Polym. Chem. 4, 470-476 (2013).
27 Yamamoto, T. et al. pi-conjugated donor-acceptor copolymers constituted of pi-excessive and pi-deficient arylene units. Optical and electrochemical properties in relation to CT structure of the polymer. J. Am. Chem. Soc. 118, 10389-10399 (1996).
28 Huo, L. J., Ye, L., Wu, Y., Li, Z. J., Guo, X., Zhang, M. J., Zhang, S. Q. & Hou, J. H. Conjugated and Nonconjugated Substitution Effect on Photovoltaic Properties of Benzodifuran-Based Photovoltaic Polymers. Macromolecules 45, 6923-6929 (2012).
29 Huo, L. J., Hou, J. H., Chen, H. Y., Zhang, S. Q., Jiang, Y., Chen, T. L. & Yang, Y. Bandgap and Molecular Level Control of the Low-Bandgap Polymers Based on 3,6-Dithiophen-2-yl-2,5-dihydropyrrolo[3,4-c]pyrrole-1,4-dione toward Highly Efficient Polymer Solar Cells. Macromolecules 42, 6564-6571 (2009).
30 Cornil, J., Gueli, I., Dkhissi, A., Sancho-Garcia, J. C., Hennebicq, E., Calbert, J. P., Lemaur, V., Beljonne, D. & Bredas, J. L. Electronic and optical properties of polyfluorene and fluorene-based copolymers: A quantum-chemical characterization. J. Chem. Phys. 118, 6615-6623 (2003).
31 Murphy, J. E., Beard, M. C., Norman, A. G., Ahrenkiel, S. P., Johnson, J. C., Yu, P. R., Micic, O. I., Ellingson, R. J. & Nozik, A. J. PbTe colloidal nanocrystals: Synthesis, characterization, and multiple exciton generation. J. Am. Chem. Soc. 128, 3241-3247 (2006).
32 Williams, A. T. R., Winfield, S. A. & Miller, J. N. Relative Fluorescence Quantum Yields Using a Computer-Controlled Luminescence Spectrometer. Analyst 108, 1067-1071 (1983).
33 Liu, Z., Tabakman, S. M., Chen, Z. & Dai, H. J. Preparation of carbon nanotube bioconjugates for biomedical applications. Nat. Protoc. 4, 1372-1382 (2009).
34 Hong, G. S., Tabakman, S. M., Welsher, K., Chen, Z., Robinson, J. T., Wang, H. L., Zhang, B. & Dai, H. J. Near-Infrared-Fluorescence-Enhanced Molecular Imaging of Live Cells on Gold Substrates. Angew. Chem. Int. Ed. 50, 4644-4648 (2011).
35 Riccabona, M., Nelson, T. R., Pretorius, D. H. & Davidson, T. E. Distance and Volume Measurement Using 3-Dimensional Ultrasonography. Journal of Ultrasound in Medicine 14, 881-886 (1995).
36 Tomlins, P. H. & Wang, R. K. Theory, developments and applications of optical coherence tomography. J. Phys. D Appl. Phys. 38, 2519-2535 (2005).
37 Wladimiroff, J. W., Tonge, H. M. & Stewart, P. A. Doppler Ultrasound Assessment of Cerebral Blood-Flow in the Human-Fetus. Brit. J. Obstet. Gynaec. 93, 471-475 (1986).
38 Lee, S., Vinegoni, C., Feruglio, P. F., Fexon, L., Gorbatov, R., Pivoravov, M., Sbarbati, A., Nahrendorf, M. & Weissleder, R. Real-time in vivo imaging of the beating mouse heart at microscopic resolution. Nat. Commun. 3 (2012).
39 Li, G., Shrotriya, V., Huang, J. S., Yao, Y., Moriarty, T., Emery, K. & Yang, Y. High-efficiency solution processable polymer photovoltaic cells by self-organization of polymer blends. Nat. Mater. 4, 864-868 (2005).
40 Burroughes, J. H., Bradley, D. D. C., Brown, A. R., Marks, R. N., Mackay, K., Friend, R. H., Burns, P. L. & Holmes, A. B. Light-Emitting-Diodes Based on Conjugated Polymers. Nature 347, 539-541 (1990).
41 Kawamura, Y., Yanagida, S. & Forrest, S. R. Energy transfer in polymer electrophosphorescent light emitting devices with single and multiple doped luminescent layers. J. Appl. Phys. 92, 87-93 (2002).
42 Facchetti, A. pi-Conjugated Polymers for Organic Electronics and Photovoltaic Cell Applications. Chem. Mater. 23, 733-758 (2011).
43 Ong, B. S., Wu, Y. L., Liu, P. & Gardner, S. High-performance semiconducting polythiophenes for organic thin-film transistors. J. Am. Chem. Soc. 126, 3378-3379 (2004).
44 Ghoroghchian, P. P., Frail, P. R., Susumu, K., Blessington, D., Brannan, A. K., Bates, F. S., Chance, B., Hammer, D. A. & Therien, M. J. Near-infrared-emissive polymersomes: Self-assembled soft matter for in vivo optical imaging. P. Natl. Acad. Sci. USA 102, 2922-2927 (2005).
45 Jin, Y. H., Ye, F. M., Zeigler, M., Wu, C. F. & Chiu, D. T. Near-Infrared Fluorescent Dye-Doped Semiconducting Polymer Dots. ACS Nano 5, 1468-1475 (2011).
46 Pu, K. Y., Li, K. & Liu, B. A Molecular Brush Approach to Enhance Quantum Yield and Suppress Nonspecific Interactions of Conjugated Polyelectrolyte for Targeted Far-Red/Near-Infrared Fluorescence Cell Imaging. Adv. Funct. Mater. 20, 2770-2777 (2010).
47 Pu, K. Y. & Liu, B. Fluorescent Conjugated Polyelectrolytes for Bioimaging. Adv. Funct. Mater. 21, 3408-3423 (2011).
48 Wang, Y. L., Peng, Q., Hou, Q. F., Zhao, K., Liang, Y. & Li, B. L. Tuning the electronic structures and optical properties of fluorene-based donor-acceptor copolymers by changing the acceptors: a theoretical study. Theoretical Chemistry Accounts 129, 257-270 (2011).
49 Wiesmann, F., Szimtenings, M., Frydrychowicz, A., Illinger, R., Hunecke, A., Rommel, E., Neubauer, S. & Haase, A. High-resolution MRI with cardiac and respiratory gating allows for accurate in vivo atherosclerotic plaque visualization in the murine aortic arch. Magnet. Reson. Med. 50, 69-74 (2003).
50 Gioux, S., Ashitate, Y., Hutteman, M. & Frangioni, J. V. Motion-gated acquisition for in vivo optical imaging. J. Biomed. Opt. 14 (2009).
51 Li, P., Yin, X., Shi, L., Rugonyi, S. & Wang, R. K. K. In vivo functional imaging of blood flow and wall strain rate in outflow tract of embryonic chick heart using ultrafast spectral domain optical coherence tomography. J. Biomed. Opt. 17 (2012).
52 An, L., Guan, G. Y. & Wang, R. K. K. High-speed 1310 nm-band spectral domain optical coherence tomography at 184,000 lines per second. J. Biomed. Opt. 16 (2011).
53 Gill, R. W. Measurement of Blood-Flow by Ultrasound—Accuracy and Sources of Error. Ultrasound Med. Biol. 11, 625-641 (1985).
54 Xuan, J. W. et al. Functional neoangiogenesis imaging of genetically engineered mouse prostate cancer using three-dimensional power Doppler ultrasound. Cancer Res. 67, 2830-2839 (2007).
55 Adler, D. C., Ko, T. H. & Fujimoto, J. G. Speckle reduction in optical coherence tomography images by use of a spatially adaptive wavelet filter. Opt. Lett. 29, 2878-2880 (2004).
56 Abd-Elmoniem, K. Z., Youssef, A. B. M. & Kadah, Y. M. Real-time speckle reduction and coherence enhancement in ultrasound imaging via nonlinear anisotropic diffusion. IEEE T Bio. Med. Eng. 49, 997-1014 (2002).
57 Hong, G., Lee, J. C., Jha, A., Diao, S., Nakayama, K. H., Hou, L., Doyle, T. C., Robinson, J. T., Antaris, A. L., Dai, H., Cooke, J. P. & Huang, N. F. Near-Infrared II Fluorescence for Imaging Hindlimb Vessel Regeneration with Dynamic Tissue Perfusion Measurement. Circ. Cardiovasc. Imaging, 10.1161/CIRCIMAGING.1113.000305 (2014).
58 Lin, W. C., Wu, C. C., Huang, T. C., Lin, W. C., Chiu, B. Y. C., Liu, R. S. & Lin, K. P. Red Blood Cell Velocity Measurement in Rodent Tumor Model: An in vivo Microscopic Study. J. Med. Biol. Eng. 32, 97-102 (2012).
59 An, L., Qin, J. & Wang, R. K. Ultrahigh sensitive optical microangiography for in vivo imaging of microcirculations within human skin tissue beds. Opt. Express 18, 8220-8228 (2010).
60 Tian, Z. Y., Yu, J. B., Wu, C. F., Szymanski, C. & McNeill, J. Amplified energy transfer in conjugated polymer nanoparticle tags and sensors. Nanoscale 2, 1999-2011 (2010).
61 Schrier, R. W. & Abraham, W. T. Mechanisms of disease—Hormones and hemodynamics in heart failure. N. Engl. J. Med. 341, 577-585 (1999).
62 Liang, Y. Y., Feng, D. Q., Wu, Y., Tsai, S. T., Li, G., Ray, C. & Yu, L. P. Highly Efficient Solar Cell Polymers Developed via Fine-Tuning of Structural and Electronic Properties. J. Am. Chem. Soc. 131, 7792-7799 (2009).
63 Huo, L. J., Huang, Y., Fan, B. H., Guo, X., Jing, Y., Zhang, M. J., Li, Y. F. & Hou, J. H. Synthesis of a 4,8-dialkoxy-benzo[1,2-b:4,5-b′]difuran unit and its application in photovoltaic polymer. Chem. Commun. 48, 3318-3320 (2012).
64 Chen, X. W., Liu, B., Zou, Y. P., Xiao, L., Guo, X. P., He, Y. H. & Li, Y. F. A new benzo[1,2-b:4,5-b′]difuran-based copolymer for efficient polymer solar cells. J. Mater. Chem. 22, 17724-17731 (2012).
65 Wurth, C., Grabolle, M., Pauli, J., Spieles, M. & Resch-Genger, U. Relative and absolute determination of fluorescence quantum yields of transparent samples. Nat. Protoc. 8, 1535-1550 (2013).
66 Keller, D. Reconstruction of Stm and Afm Images Distorted by Finite-Size Tips. Surf. Sci. 253, 353-364 (1991).
67 Matthes, R. et al. Revision of guidelines on limits of exposure to laser radiation of wavelengths between 400 nm and 1.4 mu m. Health Physics 79, 431-440 (2000).

### Example 3

### Brief Introduction:

To date, brain imaging has largely relied on X-ray computed tomography and magnetic resonance angiography with limited spatial resolution and long scanning times. Fluorescence-based brain imaging in the visible and traditional near-infrared regions (400-900 nm) is an alternative but currently requires craniotomy, cranial windows and skull thinning techniques, and the penetration depth is limited to 1-2 mm due to light scattering. Here, we report through-scalp and through-skull fluorescence imaging of mouse cerebral vasculature without craniotomy utilizing the intrinsic photoluminescence of single-walled carbon nanotubes in the 1.3-1.4 micrometer near-infrared window. Reduced photon scattering in this spectral region allows fluorescence imaging reaching a depth of >2 mm in mouse brain with sub-10 micrometer resolution. An imaging rate of ˜5.3 frames/s allows for dynamic recording of blood perfusion in the cerebral vessels with sufficient temporal resolution, providing real-time assessment of blood flow anomaly in a mouse middle cerebral artery occlusion stroke model.

### Introduction:

The essential functions of the brain dictate that any significant cerebral dysfunction or disease, such as the cerebrovascular disease, could lead to severe morbidity or mortality.¹ Brain imaging has relied on X-ray computed tomography (CT) and magnetic resonance angiography (MRA) to reach sufficient penetration depth,²⁻⁴ but these modalities are limited in spatial resolution (˜sub-millimeter) and long scanning times (˜minutes),⁵⁻⁷ impairing the resolution of small vessels and dynamic blood flow in the brain. On the other hand, fluorescence-based optical imaging of the brain in the visible and traditional near-infrared regions (400-900 nm) has relied on craniotomy, cranial windows and skull thinning techniques⁸⁻¹¹ and the penetration depth for in vivo brain imaging has been limited to a depth of 1-2 mm^{8,12-14} by light scattering.^{15,16}

Recently, we and others have shown that biological imaging with carbon nanotube and quantum dot fluorescent agents in the long near-infrared region (1.0-1.7 μm, named NIR-II region) can benefit from reduced tissue scattering and autofluorescence, reaching deeper penetration depths in vivo than traditional NIR (NIR-I, 750-900 nm) imaging techniques.¹⁷⁻²⁹ Photon scattering scales as λ^{-α}, where α=0.2 to 4 for biological tissues. The reduced scattering in the NIR-II region has allowed in vivo fluorescence imaging of blood vessels in the hindlimb of mice with ˜30 μm lateral resolution at >1 mm depth.²² Here, we report the first non-invasive brain imaging in a narrow 1.3-1.4 μm region (named as the NIR-IIa region), allowing penetration through intact scalp and skull and resolving cerebral vasculatures with a previously unattainable spatial resolution of sub-10 μm at a depth of >2 mm underneath the surface of the scalp skin in an epifluorescence imaging mode. The 1.3-1.4 μm NIR-IIa window is rationally chosen for in vivo imaging so as to minimize scattering by rejecting shorter wavelength photons than 1.3 μm while avoiding increase light absorption by water vibrational overtone modes above ˜1.4 μm. Further, dynamic NIR-IIa cerebrovascular imaging with high temporal resolution (<200 ms/frame) was used to reveal drastically reduced blood flow due to arterial occlusion in an acute stroke model on mice. Results:

Phantom Imaging in NIR-I, NIR II and NIR-IIa Regions.

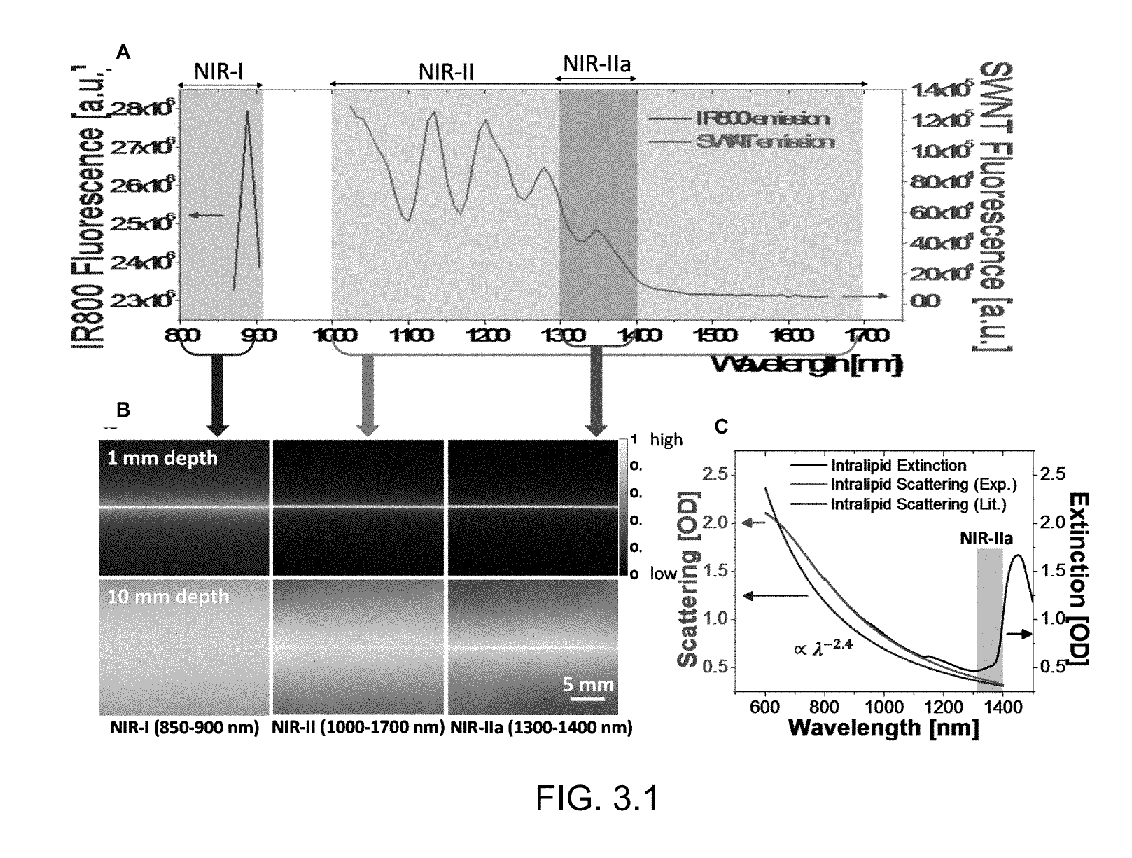

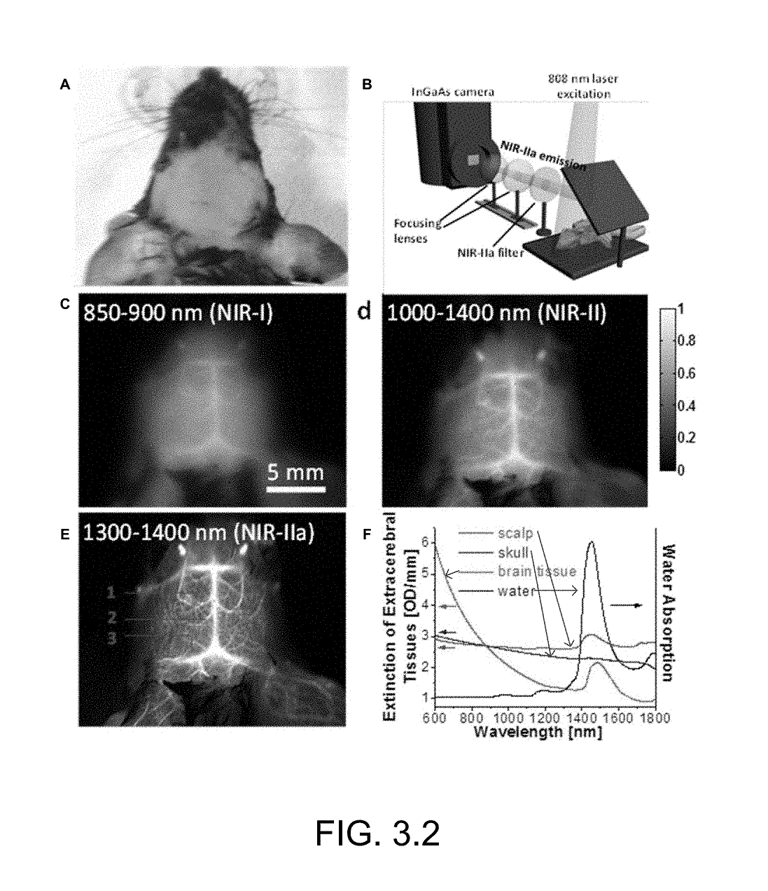

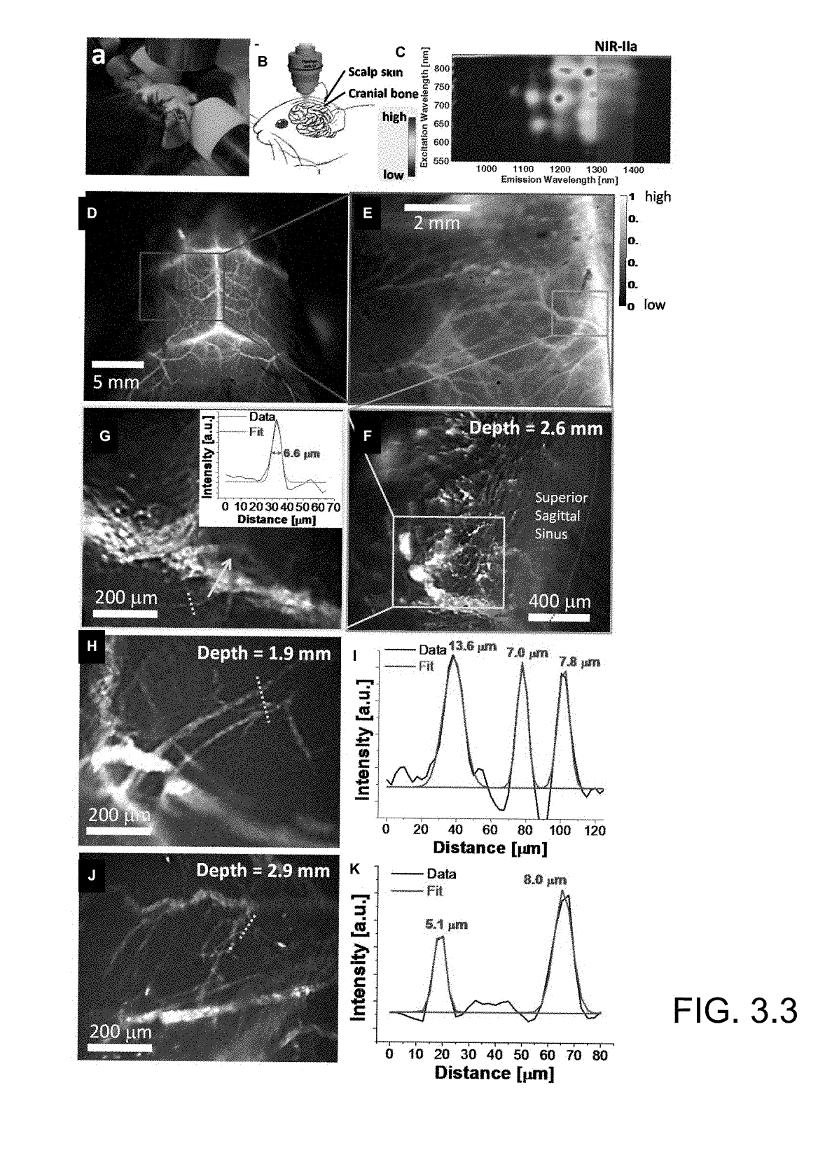

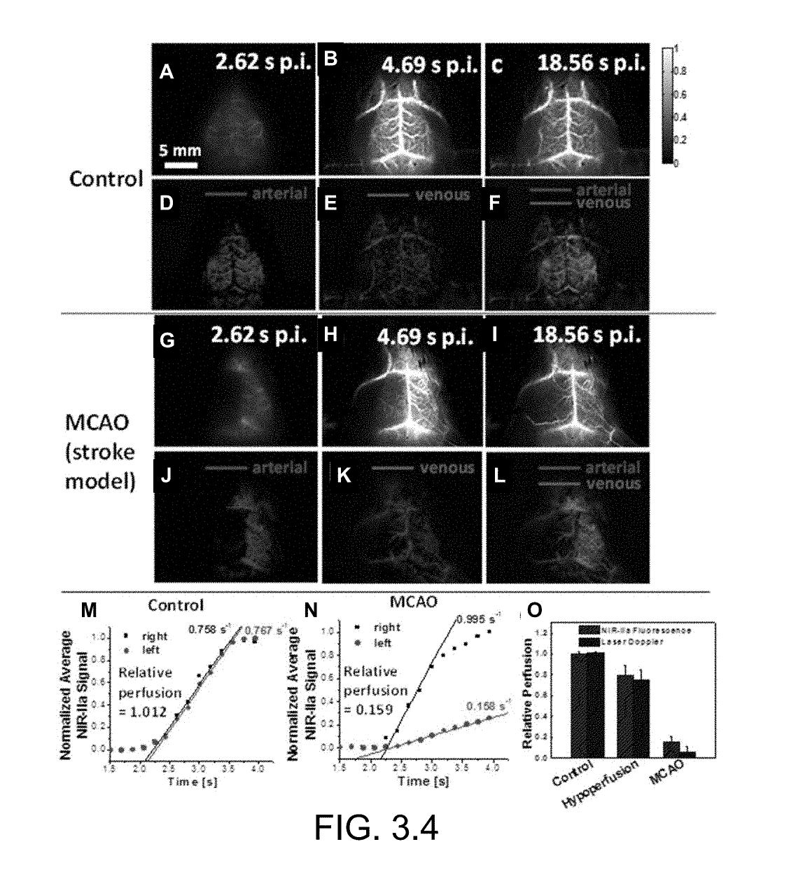

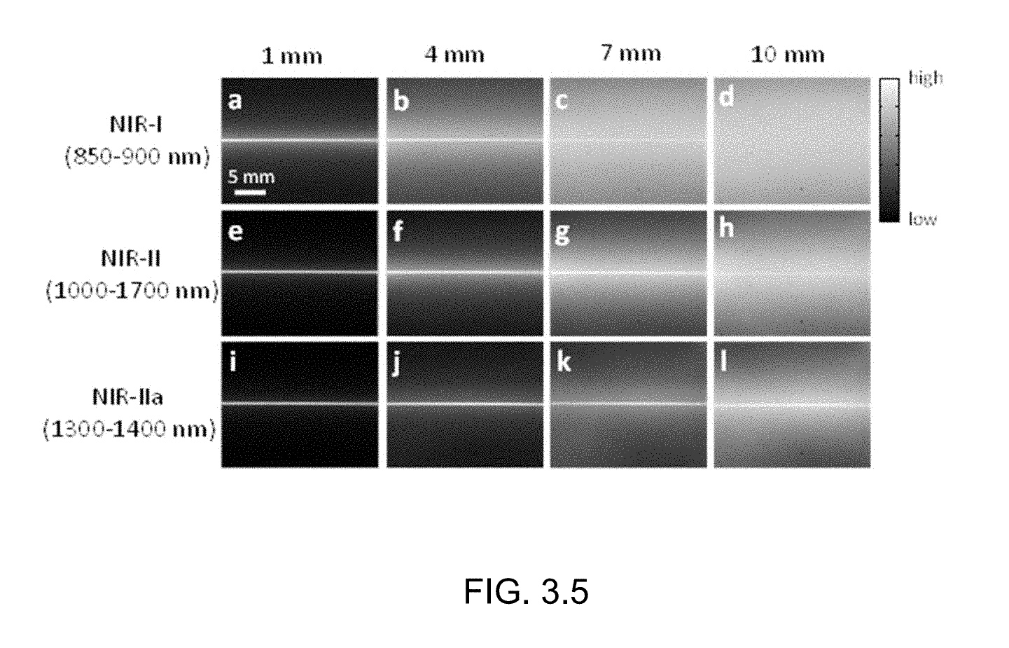

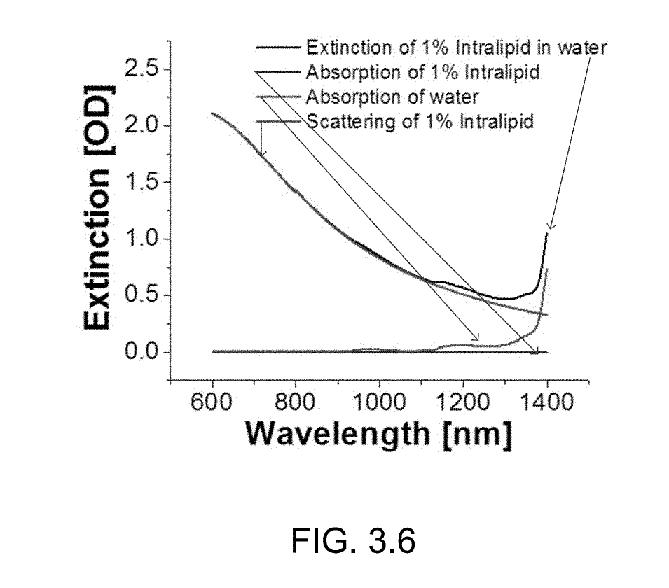

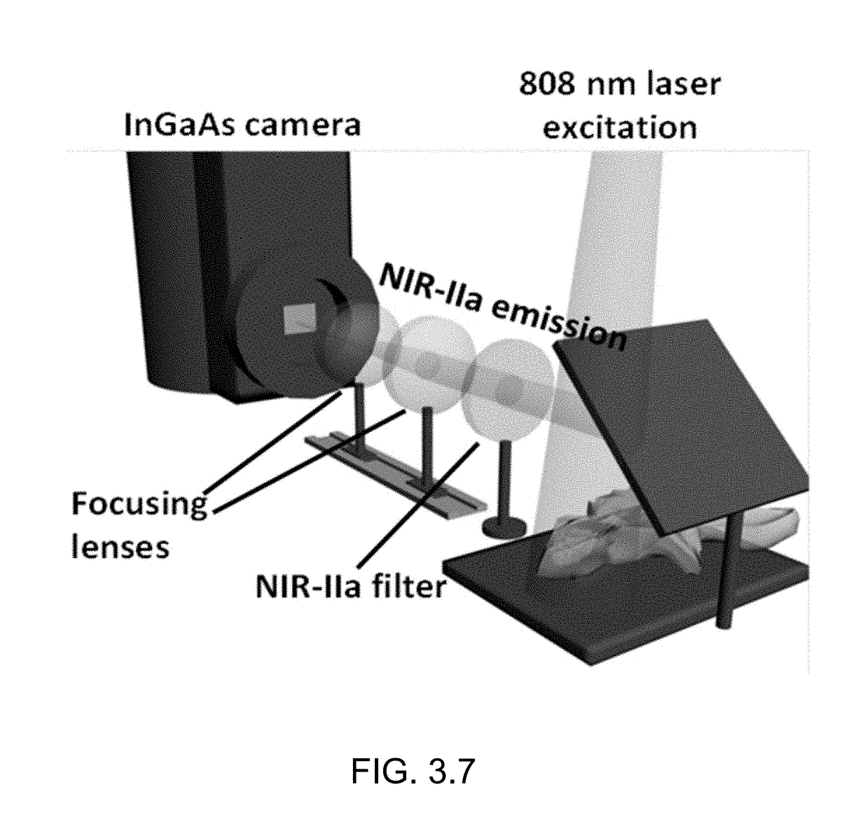

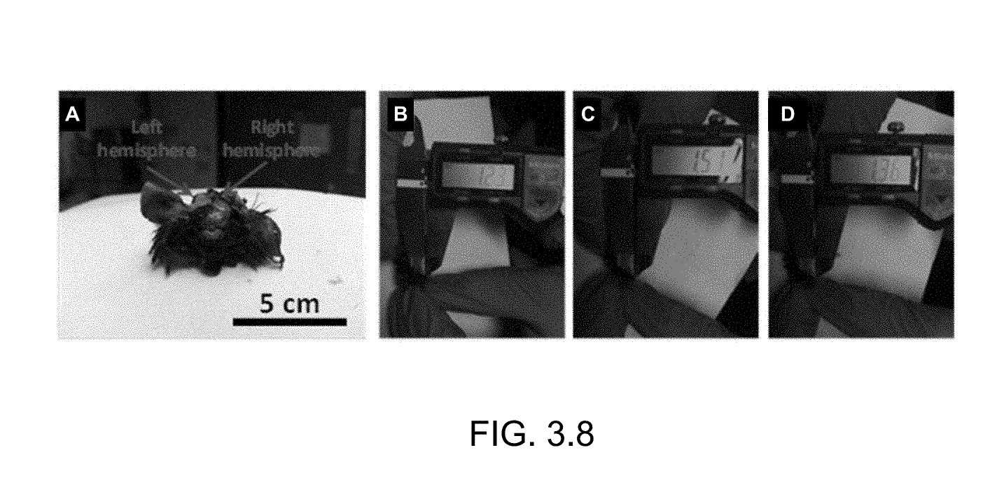

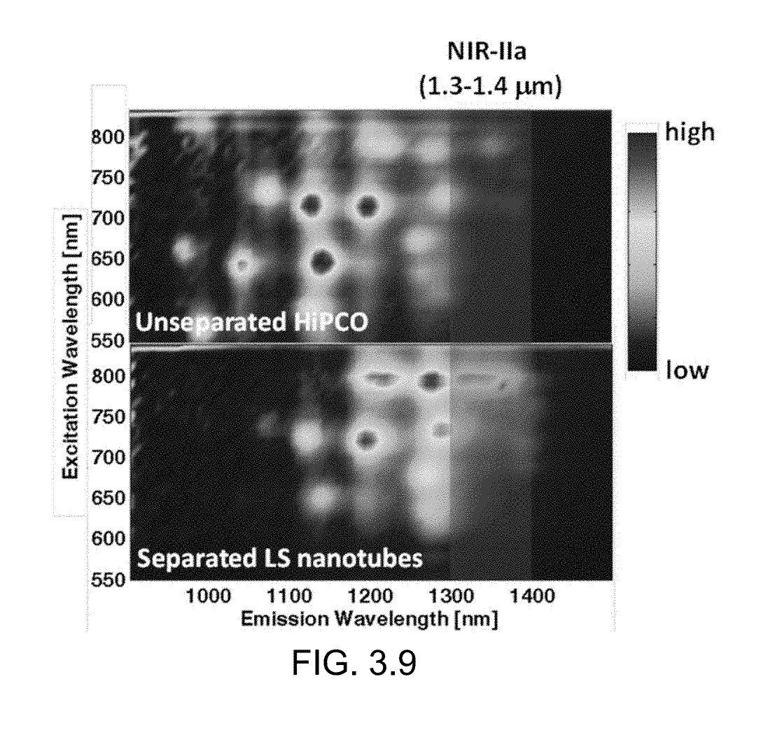

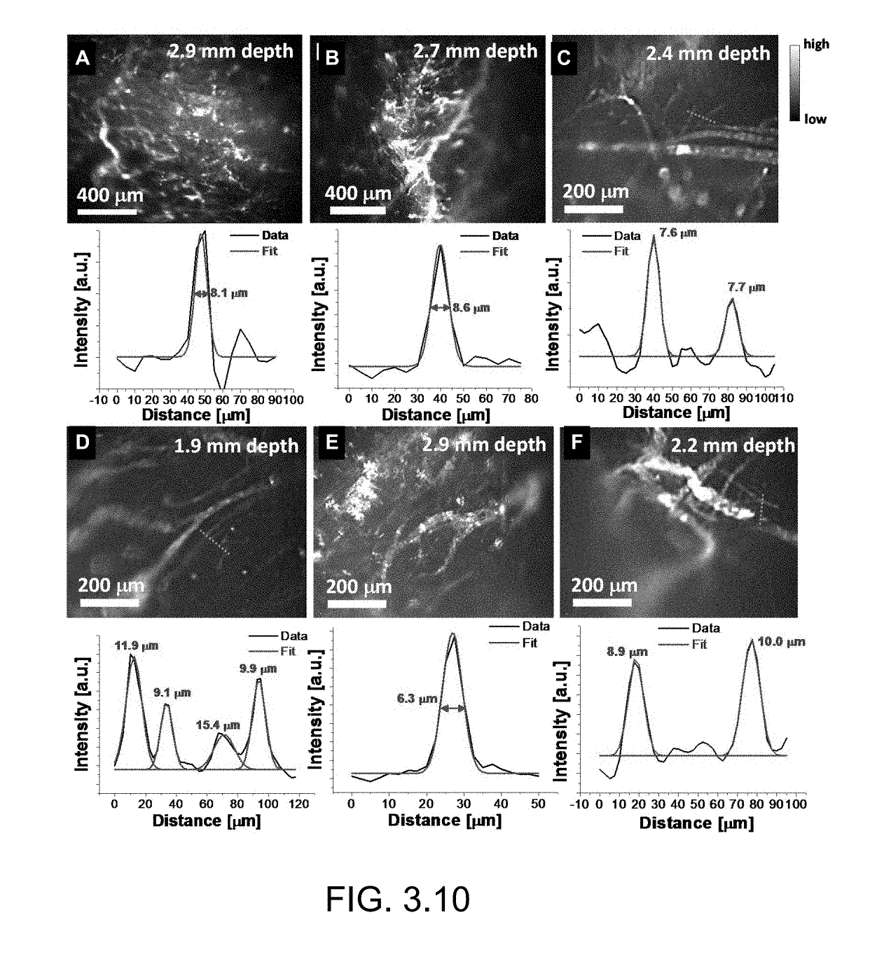

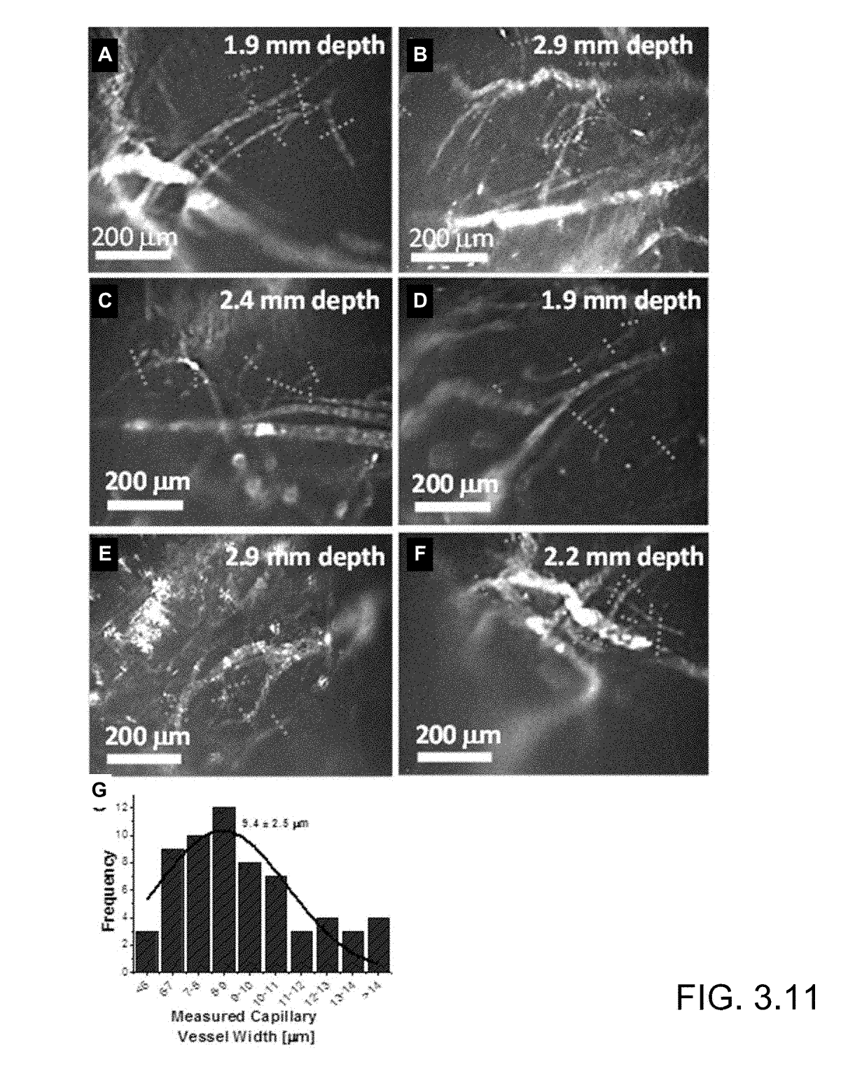

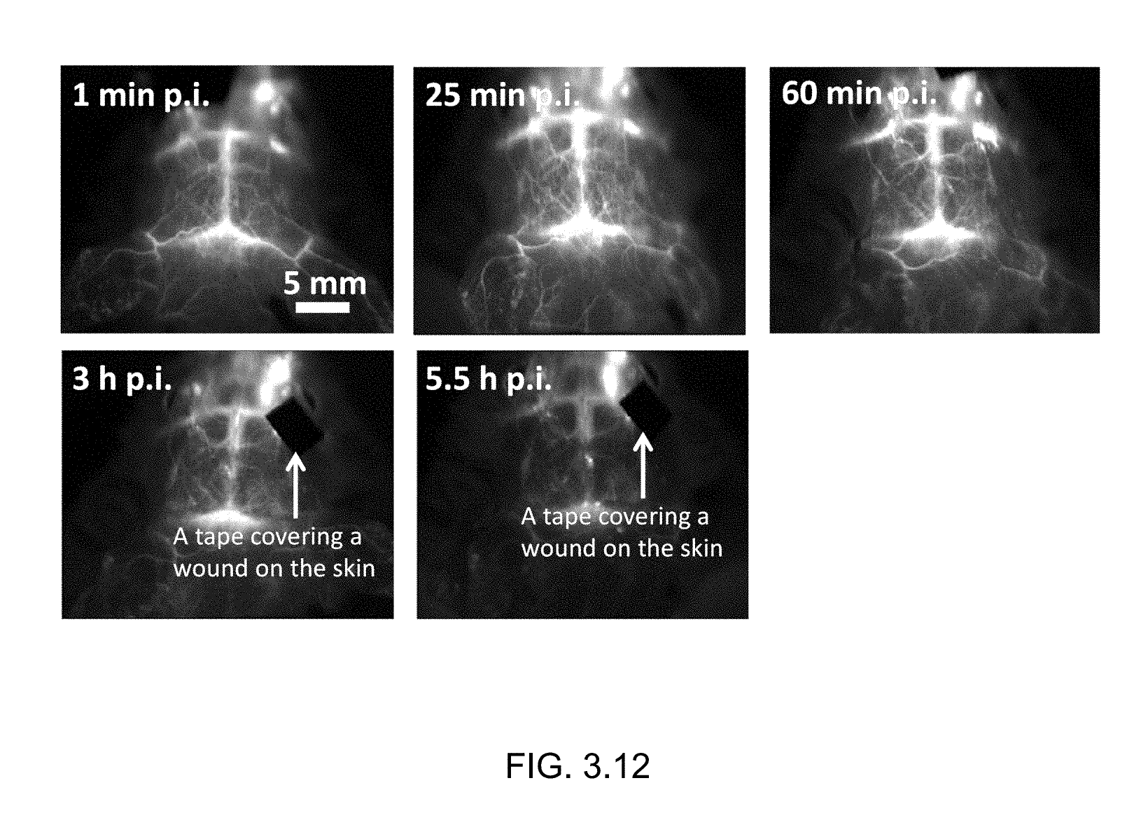

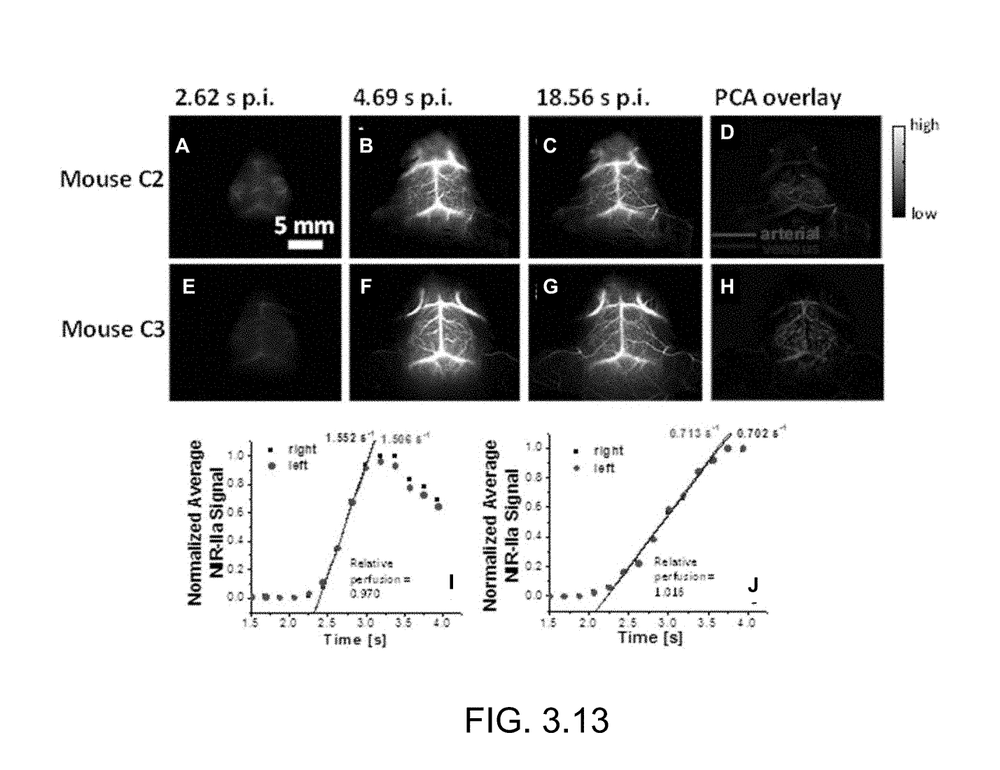

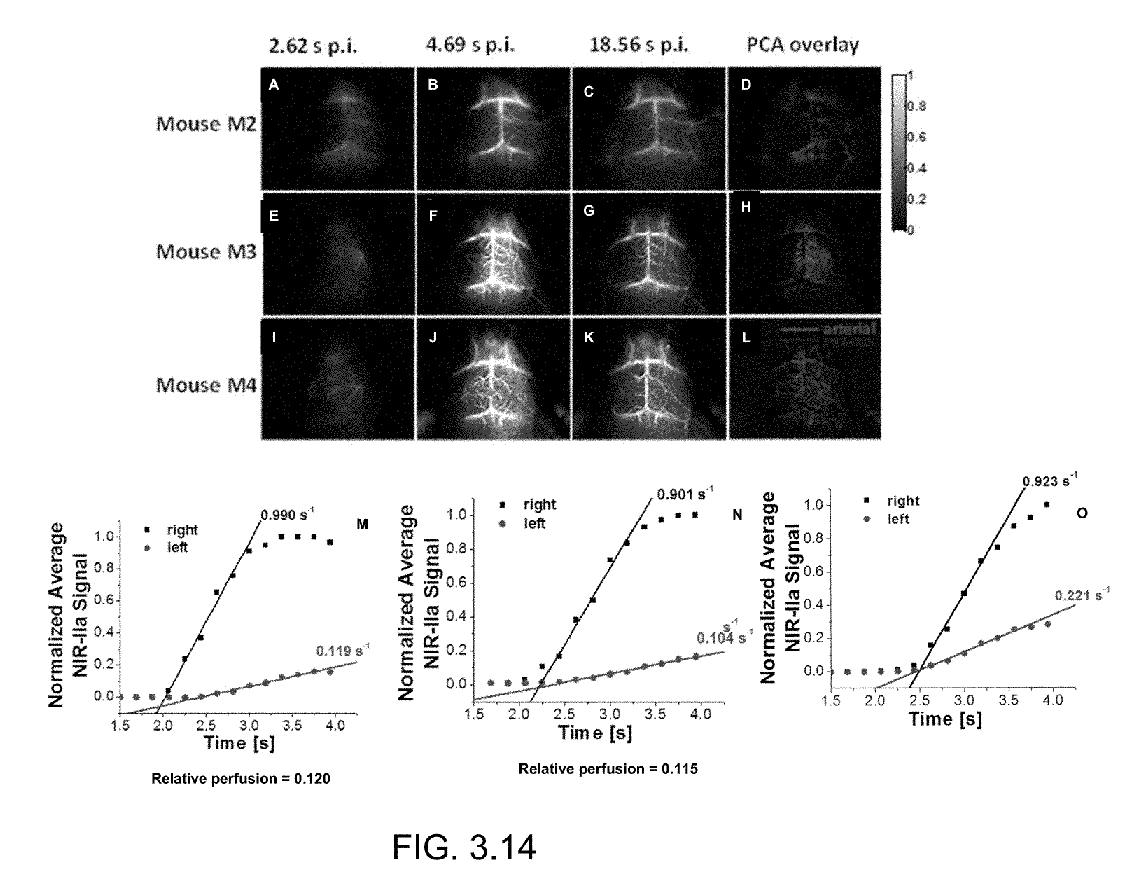

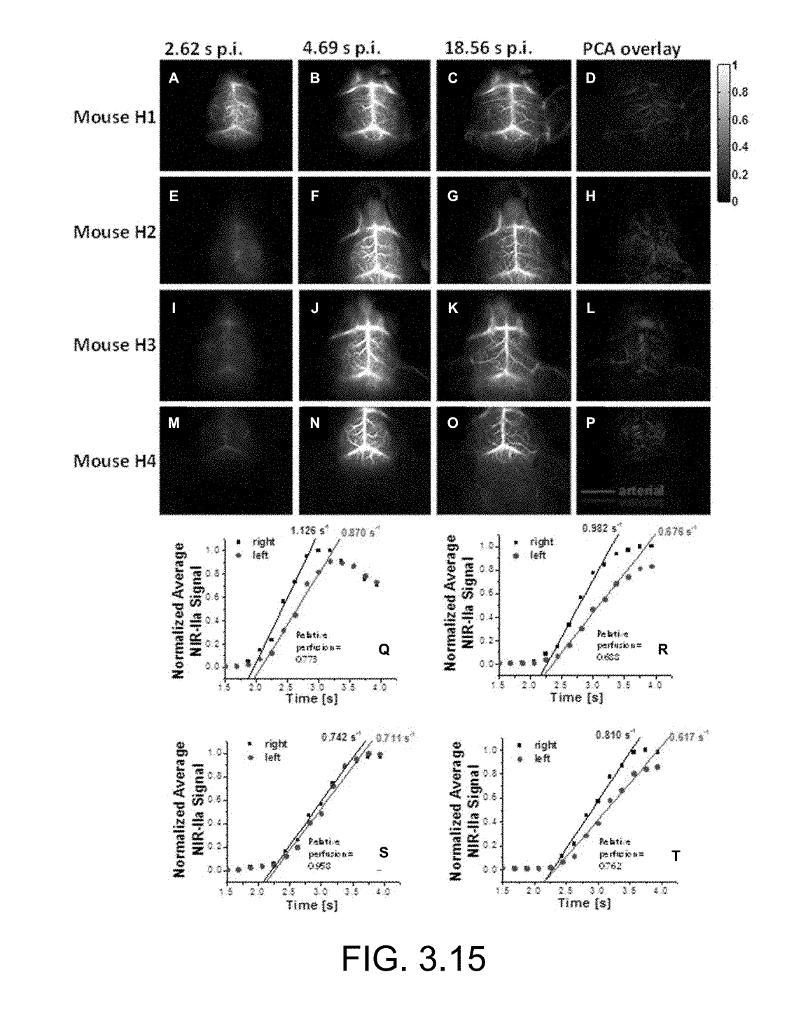

We first used a highly scattering medium (Intralipid®, 1 wt % aqueous solution) as a mimic to biological tissue²⁰ and studied the penetration depths of fluorescence imaging in the NIR-I (<900 nm), NIR-II (1.0-1.7 μm) and NIR-IIa (1.3-1.4 μm) regions. High pressure CO conversion (HiPCO) single-walled carbon nanotube³⁰ (SWNT)-IRDye800 conjugates excited by an 808-nm laser were used as emitters fluorescing in a wide 800-1700 nm span (for side-by-side comparing imaging with IRDye800 that emits in the 800-900 nm NIR-I window, and with SWNT that emits in the 1000-1700 nm NIR-II window),^{31,32} using optical filters to select detection and imaging wavelength ranges (FIG. 3.1A). When a capillary tube filled with the SWNT-IRDye800 solution was immersed in an Intralipid solution at 1 mm depth, sharp images were obtained in all three spectral windows (FIG. 3.1B, top). However, when the sample immersion depth increased to 1 cm in Intralipid, scattering of photons became obvious in the entire NIR-II (1.0-1.7 μm) region and was more severe in the NIR-I (<900 nm) region, causing the image of the capillary tube to be smeared and invisible (FIG. 3.1B, bottom). In contrast, an image taken in the 1.3-1.4 μm NIR-IIa region resolved sharp edges of the capillary tube even at 1 cm immersion depth in Intralipid (FIG. 3.1B bottom right image), suggesting reduced scattering. The observed wavelength dependence of phantom imaging (FIGS. 3.1B & 3.5) could be rationalized by considering the measured scattering coefficient vs. wavelength for 1% Intralipid (red curve, FIG. 3.1C, in agreement with previously reported reduced scattering coefficient of μ′ₛ=1.6λ^{-2.4}, blue curve. In this case, and those to follow, μ′ₛ is the scattering coefficient in mm⁻¹ and λ is the wavelength in μm, with the prefactor in appropriate unit to make units consistent on both sides of the equation).³³ The curves exhibited photon scattering scaling inversely proportional with wavelength. Rejecting shorter wavelength photons below 1.3 μm led to drastically improved imaging by avoiding feature smearing and background signals caused by scattering of short-wavelength photons. The increased absorption in the >1.4-μm region (peak shown in FIG. 3.1C) was due to light excitation of the first overtone vibration of water molecules in the Intralipid solution (FIGS. 3.1C and 3.6)³⁴ and was also excluded from the NIR-IIa imaging window.

Live Mouse Brain Imaging in NIR-I, NIR-II and NIR-IIa Regions.

We performed non-invasive in vivo cerebrovascular fluorescence imaging of healthy C57Bl/6 mice with the same SWNT-IRDye800 conjugates in several spectral windows. A solution of SWNT-IRDye800 was injected intravenously through the tail (see Methods) of a mouse. The head of the mouse with hair shaved off (FIG. 3.2A) was imaged without craniotomy in the NIR-I (<900 nm), NIR-II (1.0-1.7 □m) and NIR-IIa (1.3-1.4 μm) regions respectively (pixel size=78 μm) under an 808-nm laser illumination (FIG. 3.7). In contrast to the blurry vasculatures imaged in the <900 nm NIR-I region (FIG. 3.2B), much sharper images were obtained by detecting longer wavelength photons (FIG. 3.2C), and the sharpest and highest resolution images of brain vessels were seen in the 1.3-1.4 μm NIR-IIa region (FIG. 3.2D). Similar to the Intralipid phantom imaging case, rejecting the shorter wavelength <1.3 μm photons significantly reduced background signals and vessel blurriness caused by scattering, and improved signal/background ratio and imaging quality. The cerebral angiogram taken in the 1.3-1.4 μm NIR-IIa region clearly showed the inferior cerebral vein (labeled as 1), the superior sagittal sinus (labeled as 2) and transverse sinus (labeled as 3) at ˜1-2 mm depths under the scalp skin (FIG. 3.8), along with other cortical vessels in both cerebral hemispheres (FIG. 3.2D). A 360-degree rotational view of the mouse head was also recorded in the 1.3-1.4 μm NIR-IIa region to show a 3D perspective of the mouse head.

The wavelength dependence of fluorescence imaging of mouse brain can be gleaned by inspecting the extinction spectra of the scalp skin and the cranial bone (i.e., the skull), both of which feature a water overtone vibration absorption peak above ˜1.4 μm and a declining photon scattering profile (FIG. 3.2E). To examine the effect of scattering, we plotted reduced scattering coefficients μ′ₛ of these tissues versus wavelength λ. (FIG. 3.2F), based on the empirical formulas given in previous literature (μ′ₛ(scalp)=0.11λ⁻⁴+1.61λ^{-0.22}, where the two terms are attributed to Rayleigh and Mie scattering respectively; μ′ₛ(skull)=1.72λ^{-0.65}, and μ′ₛ is (brain tissue)=4.72λ^{-2.07})^{8,35,36} It can be seen from the plotted scattering profiles that the reduced photon scattering coefficients in the NIR-IIa range (1.54 mm⁻¹ for scalp and 1.42 mm⁻¹ for skull at 1350 nm) were lower than in the traditional NIR-I window (1.96 mm⁻¹ for scalp and 1.99 mm⁻¹ for skull at 800 nm). A simple estimate suggested that the lower scattering coefficients in NIR-IIa corresponded to ˜47% fewer scattered photons through the scalp and skull than in the NIR-I region based on the measured scalp thickness of 0.7 mm and the measured skull thickness of 0.6 mm. These factors led to drastically improved imaging of mouse cortical vessels underneath the cranial bone in the NIR-IIa region (FIG. 3.2D vs. 3.2B).

High Resolution Cerebral Imaging in the NIR-IIa Window.

We then used microscopic objectives to image cerebral vessels with high resolution at 15-fold (pixel size ˜5 μm) and 30-fold (pixel size ˜2.5 μm) higher magnification than used for FIG. 3.2. A home-made stereotactic platform was used to eliminate motion of the mouse head and a 3D translational stage with digital readout allowed us to measure the imaging focal depth inside the brain (FIGS. 3.3A & B). Further, chemical separation of large-diameter semiconducting (LS) nanotubes in the HiPCO material (see Methods for separation details) was performed and the LS tubes with fluorescence emission biased towards the favorable 1.3-1.4 μm region were used for brain imaging to optimize signal on the per unit mass basis of the injected nanotube dose (FIGS. 3.3C and 3.9). We imaged the entire mouse head (FIG. 3.3D), zoomed into its left hemisphere (FIG. 3.3E) and then performed microscopic imaging near the location of a cortical vessel branching from the superior sagittal sinus. A typical microscopic image of the brain vessels taken in the 1.3-1.4 μm NIR-IIa region focused at a depth of 2.6 mm below the surface of the scalp (FIG. 3.3F; depth of focal plane was determined by the axial travel distance of the digitally controlled stage relative to the objective, as described in Methods) revealed many tiny capillary vessels branching from the larger vessels. Cross-sectional intensity profile of one of the capillary vessels in FIG. 3.3G showed a Gaussian-fitted full width at half maximum (FWHM) of 6.6 μm (FIG. 3.3G inset). Based on our ex vivo measurement of the total thickness of scalp skin, skull and the meninges as ˜1.3 mm (FIG. 3.8), this capillary vessel located at a depth of 2.6 mm was ˜1.3 mm deep (2.6 mm-1.3 mm) within the brain tissue. This represented the highest resolution non-invasive fluorescence imaging of brain capillary vessels reported to date (see FIG. 3.3H-K and FIG. 3.10 for more brain capillaries imaged with 3 different mice at 1-3 mm depths).

Statistical analysis of measured capillary vessel widths revealed an average vessel width of 9.4±2.5 μm, based on 63 different capillary vessels with widths ranging from 5 μm to 15 μm taken from a series of microscopic cerebral vascular images (FIG. 3.11). The brain imaging depth was limited to <3 mm for our wide-field epifluorescence microscopic imaging setup. This limit was likely set by the interference from out-of-focus signals, most notably fluorescence from foreground vessels on the path of the laser beam prior to the focal plane.′ We envisage a two-photon fluorescence microscope with NIR-IIa excitation should solve this problem and generate optically sectioned images with three-dimensional reconstruction of capillary networks to even deeper penetration depths in the brain, using traditional fluorescent dyes excitable around 600-700 nm in the one-photon absorption process.

Dynamic NIR-IIa Fluorescence Imaging of Cerebral Blood Perfusion.

Immediately after tail-vein injection of SWNTs into a healthy control mouse (Mouse C1), we performed dynamic brain imaging (frame rate=5.3 frame per second) and observed the NIR-IIa signal arising from the lateral sulcus on both sides of the cerebrum within 3 s (FIG. 3.4A), before the interior cerebral vein, the superior sagittal sinus and the transverse sinus started showing up after 4 s owing to the outflow of SWNTs into the venous vessels (FIG. 3.4B&C). Principal component analysis (PCA) was applied to the time-course images over a time course of ˜20 s and discriminated the arterial vessels (red, FIG. 3.4D) from the venous vessels (blue, FIG. 3.4E) based on their haemodynamic difference (FIG. 3.4F).^{20,22} The injected SWNTs kept circulating in the blood for at least 3 h post injection, allowing for static imaging in this time window (FIG. 3.12). We then repeated the study in mice with surgically induced middle cerebral artery occlusion (MCAO) as a model for stroke to the left cerebral hemisphere. After injection of an SWNT solution into a mouse with MCAO (Mouse M1), the right hemisphere exhibited very similar blood flow to the healthy Mouse C1, while the left hemisphere with MCAO showed a marked delay of blood flow revealed by NIR-IIa fluorescence (FIG. 3.4G-I). PCA analysis of Mouse M1 revealed a more extended venous vessel network (blue) in the right cerebral hemisphere than in the left, and the arterial vessels (red) only showed up in the intact right hemisphere (FIG. 3.4J-0. Similar results were reproduced with several healthy mice (FIG. 3.13A-H) and MCAO diseased mice (FIG. 3.14A-0.

The dynamic NIR-IIa fluorescence imaging allowed us to quantify cerebral blood perfusion by measuring average NIR-IIa intensity in the region of MCAs.²² In the healthy Mouse C1, the linearly rising fluorescence signals in both cerebral hemispheres had almost identical slopes, suggesting a relative perfusion of ˜1 (see SI for perfusion analysis) (FIG. 3.4M; FIG. 3.13I&J for repeat). However, in Mouse M1 with MCAO, the relative perfusion in the occluded left hemisphere was only 0.159 (FIG. 3.4N; FIG. 3.14M-0 for repeat). Another group of mice (n=4) with cerebral hypoperfusion as a model of circulatory shock was imaged (FIG. 3.15) to reveal a decrease of blood perfusion of ˜20% compared to a dramatic decrease of blood perfusion of ˜85% in the MCAO group (red bars, FIG. 3.40). The data agreed with laser Doppler measurements (blue bars, FIG. 3.40). One advantage of NIR-IIa dynamic fluorescence imaging is that it tracks blood flow in a wide-field imaging setup, which allows for simultaneous tracking of blood flow in multiple vessels and could potentially provide a complementary method to two-photon microscopy based blood flow measurement, which performs line scan along the direction of one single vessel at a time with kHz temporal resolution.³⁸⁻⁴⁰

### Discussion:

The NIR-IIa fluorescence imaging method is advantageous for brain imaging in terms of high resolution, deep penetration depth and non-invasive nature without the need for craniotomy. Previously, imaging in the traditional NIR-I region (750-900 nm) has observed sub-10 μm capillary vessels at ˜350 μm depth with craniotomy to remove scattering extracerebral tissues,¹² much shallower than the imaging depth of >2 mm below the scalp in the NIR-IIa region without craniotomy. This striking difference can be rationalized by considering the highly scattering nature of the brain tissue, with a large scattering coefficient of ˜7.49 mm⁻¹ at 800 nm versus ˜2.54 mm⁻¹ at 1350 nm (FIG. 3.2F), suggesting ˜10⁴ times greater scattered photons in NIR-I than in NIR-IIa through a ˜2 mm thick brain tissue. Two- and multi-photon microscopy techniques have reported a typical penetration depth of 1-2 mm in the brain tissue, facilitated by highly localized non-linear excitation and detection;^{8,13,14,41} but scalp and skull usually need to be removed and replaced with a cranial window,^{8,9} or polished and thinned^{10,11} to allow for deeper penetration depth. Similarly, the invasive removal of the overlying tissues such as the scalp and the skull is involved for brain angiography with laser speckle contrast imaging (LSCI)^{42,43} and optical frequency domain imaging (OFDI),⁴⁴ which have been reported to reach a high spatial resolution of ˜10 □m in vivo.^{44,45} To avoid the invasive treatment of the extracerebral tissue, optical coherence tomography (OCT) and photoacoustic microscopy have both shown the capability of visualizing cortical vasculature at the cost of spatial resolution (10-100 □m), unable to resolve sub-10 μm capillary vessels in vivo. In light of the possible inflammatory responses such as altered pial blood vessels induced by invasive extracerebral treatment,¹⁰ the NIR-IIa fluorescence based brain angiography provides a completely non-invasive method to visualize the brain vasculature in live mouse with unprecedented resolution and penetration depth.

Although the maximum penetration depth of <3 mm in this work prevents it from clinical imaging of much deeper vasculature inside the human brain, our new brain imaging method based on the long-wavelength NIR-IIa fluorescence can serve as a non-invasive biomedical imaging tool of understanding the mechanisms of the impaired neurovascular and neurometabolic regulation on both cortical and sub-cortical levels in animal model studies. Besides the cerebrovascular dynamics probed with NIR-IIa fluorescence in this work, we also envisage non-invasive brain imaging of neuronal and/or glial network activity with cellular resolution in live mice using neuronal activity indicators labeled with NIR-IIa fluorophores. In the long term, we anticipate fluorescence imaging using novel, metabolizable fluorophores with even longer emission wavelengths and less scattering to be eventually applied to diagnosis and monitor of cerebral vascular anomaly such as arteriovenous malformation and brain tumor, and other neurovascular disregulations involving rapid modification of cerebral blood flow, such as migraine.

### Conclusions:

In this work, we explored a new biological transparent sub-window in the 1.3-1.4 μm, i.e., NIR-IIa region and performed non-invasive brain imaging in this window by penetrating through the intact skin and skull. We resolved cerebral vasculatures with a high spatial resolution of sub-10 μm at a depth of >2 mm in an epifluorescence imaging mode. Compare to previous NIR-II work, we found that the 1.3-1.4 μm NIR-IIa window for in vivo imaging can further reduce tissue scattering by rejecting shorter wavelength photons than 1.3 μm. The truly non-invasive nature and dynamic imaging capability of NIR-IIa cerebrovascular imaging could allow for high spatial and temporal resolution imaging to follow biological processes in the brain at the molecular scale.

### Methods Section:

Preparation of biocompatible SWNT-IRDye800 multicolor emitter.

Raw HiPCO SWNTs (Unidym) were suspended in an aqueous solution containing 1 wt % sodium deoxycholate by 1 hour of bath sonication. This suspension was then ultracentrifuged at 300,000 g to collect the supernatant, and 0.75 mg·ml⁻¹ of DSPE-mPEG (5 kDa) (1,2-distearoyl-sn-glycero-3-phosphoethanolamine-N-[methoxy(polyethyleneglycol, 5000)], Laysan Bio) along with 0.25 mg·ml⁻¹ of DSPE-PEG (5 kDa)-NH2 (1,2-distearoyl-sn-glycero-3-phosphoethanolamine-N-[amino(polyethyleneglycol, 5000)], Laysan Bio) was added to the supernatant. The resulting suspension was sonicated for 5 min and dialyzed against 1× phosphate buffer saline (PBS) at pH 7.4. These amine-functionalized SWNTs were conjugated with IRDye800 dye molecules. Briefly, the as-made SWNT solution was concentrated down to ˜2 μM after removal of excess surfactant through 30-kDa centrifugal filters (Amicon) and was mixed with 0.1 mM IRDye800 NHS ester (LI-COR) dissolved in dimethylsulfoxide (DMSO). The reaction was allowed to proceed for 1 h at pH 7.4 before removing excess IRDye800 by filtration through the 30-kDa filters.

Sorting of LS Nanotubes.

A 1-cm diameter column filled with 20 ml of ally dextran-based size-exclusion gel (Sephacryl S-200, GE Healthcare) was used to perform diameter separation of HiPCO SWNTs. 2.5 mg of raw HiPCO SWNTs was sonicated in water with 1 wt % sodium cholate (SC) for 1 h, followed by ultracentrifugation at 300,000 g for 30 min. 80% of the supernatant was collected and diluted with same volume of 1 wt % sodium dodecyl sulfate (SDS) solution before adding to the gel column. Metallic SWNTs passed the column directly, while semiconducting SWNTs were trapped. Finally, a 0.6 wt % SC/0.4 wt % SDS mixed surfactant solution was added to wash out and collect the LS nanotubes.

NIR Fluorescence Spectroscopy of the SWNT-IRDye800 Conjugate.

NIR fluorescence spectrum was taken using a home-built NIR spectroscopy setup in the 850-1650 nm region. The excitation light was provided by a 160-mW, 808-nm diode laser (RMPC lasers) and filtered by an 850-nm short-pass filter (Thorlabs), a 1000-nm short-pass filter (Thorlabs), an 1100-nm short-pass filter (Omega) and a 1300-nm short-pass filter (Omega). The excitation light was allowed to pass through the solution of SWNT-IRDye800 conjugate in a 1-mm path cuvette (Starna Cells) and the emission was collected in the transmission geometry. The excitation light was rejected using an 850-nm long-pass filter (Thorlabs). The emitted fluorescence was directed into a spectrometer (Acton SP2300i) equipped with a liquid-nitrogen-cooled InGaAs linear array detector (Princeton OMA-V).

Mouse Handling, Surgery and Injection.

C57Bl/6 mice were obtained from Taconic Farms. All animal studies were approved by Stanford University's Administrative Panel on Laboratory Animal Care. For induction of MCAO, an incision was made on the left common carotid artery (CCA) and a silicon-coated nylon filament was introduced into the left CCA and threaded forward into internal carotid artery (ICA) until the tip occluded the origin of the middle cerebral artery (MCA). For induction of cerebral hypoperfusion, only the left external carotid artery (ECA) and common carotid artery (CCA) were ligated. The hair over the scalp was removed using Nair before tail-vein injection and imaging. All mice were anaesthetized before injection using 2 L·min⁻¹ 02 gas flow mixed with 3% Isoflurane. For comparative cerebrovascular imaging in different NIR sub-regions, a solution (200 μL) of 0.43 mg·mL⁻¹ SWNT-IRDye800 was injected. For dynamic NIR-IIa imaging, a solution (200 μL) of 0.43 mg·mL⁻¹ SWNT-DSPE-mPEG without IRDye800 was injected. A 28-gauge syringe needle was inserted into the tail vein for injection. For steady-state imaging of the mouse brain, injection was done ˜5 min before the mouse was placed on the imaging stage. For dynamic imaging, injection was done in the dark when the mouse was already placed on the imaging stage and the InGaAs camera started recording images continuously immediately after the nanotube solution was injected into the tail vein.

NIR Fluorescence Imaging in Different Sub-Regions.

A liquid-nitrogen-cooled, 320×256 pixel two-dimensional InGaAs array (Princeton Instruments) was used to take images in all sub-regions of NIR including the NIR-I, NIR-II and NIR-IIa regions. The excitation light was provided by an 808-nm diode laser coupled to a 4.5-mm collimator, and filtered by an 850-nm short-pass filter and a 1000-nm short-pass filter (Thorlabs), with an in-plane excitation power density of 140 mW·cm⁻². The emitted fluorescence was allowed to pass through different filter sets to ensure the images taken in different NIR sub-regions. A lens pair consisting of two achromats was used to focus the image onto the detector with different magnifications (see SI for detailed information for selections of filter sets and lens sets).

Microscopic NIR-IIa Imaging of Cerebral Vessels.

High-magnification intravital imaging of cerebral vessels was carried out with a 160-mW, 808-nm diode laser as the excitation source and two objective lens (4× and 10×, Bausch & Lomb) for microscopic imaging. The mouse with scalp hair removed was injected with a solution (200 μL) of 0.22 mg·mL⁻¹ LS nanotubes and placed on a home-made stereotactic platform fixed on a motorized 3D translational stage with digital position readout (Newport). The resulting NIR photoluminescence was collected using the same 2D InGaAs camera in the NIR-IIa window as aforementioned. The depth of vessels was determined by recording the distance the 3D stage had travelled axially to the objective from focusing on the scalp surface. Snell's law was applied to correct for the actual depth due to refractive index mismatch.′

Dynamic Cerebrovascular Imaging in the NIR-IIa Window.

The dynamic imaging setup was the same as the aforementioned NIR-IIa fluorescence brain imaging with lower magnification that covered the entire mouse head. The InGaAs camera was set to expose continuously with an exposure time of 100 ms, to which an 87.5-ms overhead time was added to result in a frame rate of 5.3 frames·s⁻¹. The dynamic-contrast-enhanced images were obtained by loading the first 100 consecutive frames with the MATLAB software for PCA analysis of arterial and venous vessels based on their haemodynamic difference.^{20,22,46}

### REFERENCES

1 Go, A. S. et al. Heart Disease and Stroke Statistics-2013 Update A Report From the American Heart Association. Circulation 127, E6-E245 (2013).
2 Schramm, P., Schellinger, P. D., Fiebach, J. B., Heiland, S., Jansen, O., Knauth, M., Hacke, W. & Sartor, K. Comparison of CT and CT angiography source images with diffusion-weighted imaging in patients with acute stroke within 6 hours after onset. Stroke 33, 2426-2432 (2002).
3 Wright, S. N., Kochunov, P., Mut, F., Bergamino, M., Brown, K. M., Mazziotta, J. C., Toga, A. W., Cebral, J. R. & Ascoli, G. A. Digital reconstruction and morphometric analysis of human brain arterial vasculature from magnetic resonance angiography. Neuroimage 82, 170-181 (2013).
4 Huang, C. H., Chen, C. C. V., Siow, T. Y., Hsu, S. H. S., Hsu, Y. H., Jaw, F. S. & Chang, C. High-Resolution Structural and Functional Assessments of Cerebral Microvasculature Using 3D Gas Delta R-2*-mMRA. Plos One 8 (2013).
5 Flohr, T. G. et al. First performance evaluation of a dual-source CT (DSCT) system (vol 16, pg 256, 2006). Eur Radiol 16, 1405-1405 (2006).
6 Jacoby, C., Boring, Y. C., Beck, A., Zernecke, A., Aurich, V., Weber, C., Schrader, J. & Flogel, U. Dynamic changes in murine vessel geometry assessed by high-resolution magnetic resonance angiography: A 9.4T study. J Magn Reson Imaging 28, 637-645 (2008).
7 Paulus, M. J., Gleason, S. S., Kennel, S. J., Hunsicker, P. R. & Johnson, D. K. High resolution X-ray computed tomography: An emerging tool for small animal cancer research. Neoplasia 2, 62-70 (2000).
8 Horton, N. G., Wang, K., Kobat, D., Clark, C. G., Wise, F. W., Schaffer, C. B. & Xu, C. In vivo three-photon microscopy of subcortical structures within an intact mouse brain. Nat Photonics 7, 205-209 (2013).
9 Svoboda, K., Denk, W., Kleinfeld, D. & Tank, D. W. In vivo dendritic calcium dynamics in neocortical pyramidal neurons. Nature 385, 161-165 (1997).
10 Drew, P. J., Shih, A. Y., Driscoll, J. D., Knutsen, P. M., Blinder, P., Davalos, D., Akassoglou, K., Tsai, P. S. & Kleinfeld, D. Chronic optical access through a polished and reinforced thinned skull. Nat Methods 7, 981-U960 (2010).
11 Yang, G., Pan, F., Parkhurst, C. N., Grutzendler, J. & Gan, W. B. Thinned-skull cranial window technique for long-term imaging of the cortex in live mice. Nat Protoc 5, 201-208 (2010).
12 Martirosyan, N. L., Cavalcanti, D. D., Eschbacher, J. M., Delaney, P. M., Scheck, A. C., Abdelwahab, M. G., Nakaji, P., Spetzler, R. F. & Preul, M. C. Use of in vivo near-infrared laser confocal endomicroscopy with indocyanine green to detect the boundary of infiltrative tumor Laboratory investigation. J Neurosurg 115, 1131-1138 (2011).
13 Theer, P., Hasan, M. T. & Denk, W. Two-photon imaging to a depth of 1000 microm in living brains by use of a Ti:Al2O3 regenerative amplifier. Optics letters 28, 1022-1024 (2003).
14 Kobat, D., Horton, N. G. & Xu, C. In vivo two-photon microscopy to 1.6-mm depth in mouse cortex. Journal of biomedical optics 16, 106014 (2011).
15 Splinter, R. & Hooper, B. A. An introduction to biomedical optics. (Taylor & Francis, 2007).
16 Frangioni, J. V. In vivo near-infrared fluorescence imaging. Curr Opin Chem Biol 7, 626-634 (2003).
17 Smith, A. M., Mancini, M. C. & Nie, S. Bioimaging: second window for in vivo imaging. Nature nanotechnology 4, 710-711 (2009).
18 Yi, H. J., Ghosh, D., Ham, M. H., Qi, J. F., Barone, P. W., Strano, M. S. & Belcher, A. M. M13 Phage-Functionalized Single-Walled Carbon Nanotubes As Nanoprobes for Second Near-Infrared Window Fluorescence Imaging of Targeted Tumors. Nano Lett 12, 1176-1183 (2012).
19 Welsher, K., Liu, Z., Sherlock, S. P., Robinson, J. T., Chen, Z., Daranciang, D. & Dai, H. A route to brightly fluorescent carbon nanotubes for near-infrared imaging in mice. Nature nanotechnology 4, 773-780 (2009).
20 Welsher, K., Sherlock, S. P. & Dai, H. Deep-tissue anatomical imaging of mice using carbon nanotube fluorophores in the second near-infrared window. Proceedings of the National Academy of Sciences of the United States of America 108, 8943-8948 (2011).
21 Hong, G., Wu, J. Z., Robinson, J. T., Wang, H., Zhang, B. & Dai, H. Three-dimensional imaging of single nanotube molecule endocytosis on plasmonic substrates. Nature communications 3, 700 (2012).
22 Hong, G., Lee, J. C., Robinson, J. T., Raaz, U., Xie, L., Huang, N. F., Cooke, J. P. & Dai, H. Multifunctional in vivo vascular imaging using near-infrared II fluorescence. Nature medicine 18, 1841-1846 (2012).
23 Won, N., Jeong, S., Kim, K., Kwag, J., Park, J., Kim, S. G. & Kim, S. Imaging Depths of Near-Infrared Quantum Dots in First and Second Optical Windows. Molecular imaging 11, 338-352 (2012).
24 Hong, G. S., Robinson, J. T., Zhang, Y. J., Diao, S., Antaris, A. L., Wang, Q. B. & Dai, H. J. In Vivo Fluorescence Imaging with Ag2S Quantum Dots in the Second Near-Infrared Region. Angew Chem Int Edit 51, 9818-9821 (2012).
25 Naczynski, D. J., Tan, M. C., Zevon, M., Wall, B., Kohl, J., Kulesa, A., Chen, S., Roth, C. M., Riman, R. E. & Moghe, P. V. Rare-earth-doped biological composites as in vivo shortwave infrared reporters. Nature communications 4 (2013).
26 Tao, Z. M., Hong, G. S., Shinji, C., Chen, C. X., Diao, S., Antaris, A. L., Zhang, B., Zou, Y. P. & Dai, H. J. Biological Imaging Using Nanoparticles of Small Organic Molecules with Fluorescence Emission at Wavelengths Longer than 1000 nm. Angew Chem Int Edit 52, 13002-13006 (2013).
27 Dong, B. H., Li, C. Y., Chen, G. C., Zhang, Y. J., Zhang, Y., Deng, M. J. & Wang, Q. B. Facile Synthesis of Highly Photoluminescent Ag2Se Quantum Dots as a New Fluorescent Probe in the Second Near-Infrared Window for in Vivo Imaging. Chem Mater 25, 2503-2509 (2013).
28 Hong, G., Lee, J. C., Jha, A., Diao, S., Nakayama, K. H., Hou, L., Doyle, T. C., Robinson, J. T., Antaris, A. L., Dai, H., Cooke, J. P. & Huang, N. F. Near-Infrared II Fluorescence for Imaging Hindlimb Vessel Regeneration With Dynamic Tissue Perfusion Measurement. Circulation. Cardiovascular imaging 7, 517-525 (2014).
29 Hong, G. et al. Ultrafast fluorescence imaging in vivo with conjugated polymer fluorophores in the second near-infrared window. Nature communications 5, 4206 (2014).
Chiang, I. W., Brinson, B. E., Huang, A. Y., Willis, P. A., Bronikowski, M. J., Margrave, J. L., Smalley, R. E. & Hauge, R. H. Purification and characterization of single-wall carbon nanotubes (SWNTs) obtained from the gas-phase decomposition of CO (HiPco process). J Phys Chem B 105, 8297-8301 (2001).
31 O'Connell, M. J., Bachilo, S. M., Huffman, C. B., Moore, V. C., Strano, M. S., Haroz, E. H., Rialon, K. L., Boul, P. J., Noon, W. H., Kittrell, C., Ma, J. P., Hauge, R. H., Weisman, R. B. & Smalley, R. E. Band gap fluorescence from individual single-walled carbon nanotubes. Science 297, 593-596 (2002).
32 Bachilo, S. M., Strano, M. S., Kittrell, C., Hauge, R. H., Smalley, R. E. & Weisman, R. B. Structure-assigned optical spectra of single-walled carbon nanotubes. Science 298, 2361-2366 (2002).
33 van Staveren, H. J., Moes, C. J., van Marie, J., Prahl, S. A. & van Gernert, M. J. Light scattering in Intralipid-10% in the wavelength range of 400-1100 nm. Applied optics 30, 4507-4514 (1991).
34 Curcio, J. A. & Petty, C. C. The near Infrared Absorption Spectrum of Liquid Water. J Opt Soc Am 41, 302-304 (1951).
35 Bashkatov, A. N., Genina, E. A., Kochubey, V. I. & Tuchin, V. V. Optical properties of human cranial bone in the spectral range from 800 to 2000 nm—art. no. 616310. Saratov Fall Meeting 2005: Optical Technologies in Biophysics and Medicine VII 6163, 16310-16310 (2006).
36 Bashkatov, A. N., Genina, E. A., Kochubey, V. I. & Tuchin, V. V. Optical properties of human skin, subcutaneous and mucous tissues in the wavelength range from 400 to 2000 nm. J Phys D Appl Phys 38, 2543-2555 (2005).
37 White, J. G., Amos, W. B. & Fordham, M. An Evaluation of Confocal Versus Conventional Imaging of Biological Structures by Fluorescence Light-Microscopy. J Cell Biol 105, 41-48 (1987).
38 Kleinfeld, D., Mitra, P. P., Helmchen, F. & Denk, W. Fluctuations and stimulus-induced changes in blood flow observed in individual capillaries in layers 2 through 4 of rat neocortex. Proceedings of the National Academy of Sciences of the United States of America 95, 15741-15746 (1998).
39 Shih, A. Y., Driscoll, J. D., Drew, P. J., Nishimura, N., Schaffer, C. B. & Kleinfeld, D. Two-photon microscopy as a tool to study blood flow and neurovascular coupling in the rodent brain. J Cerebr Blood F Met 32, 1277-1309 (2012).
40 Helmchen, F. & Kleinfeld, D. In Vivo Measurements of Blood Flow and Glial Cell Function with Two-Photon Laser-Scanning Microscopy. Method Enzymol 444, 231-254 (2008).
41 Helmchen, F. & Denk, W. Deep tissue two-photon microscopy. Nat Methods 2, 932-940 (2005).
42 Dunn, A. K. Laser Speckle Contrast Imaging of Cerebral Blood Flow. Ann Biomed Eng 40, 367-377 (2012).
43 Boas, D. A. & Dunn, A. K. Laser speckle contrast imaging in biomedical optics. Journal of biomedical optics 15 (2010).
44 Vakoc, B. J., Lanning, R. M., Tyrrell, J. A., Padera, T. P., Bartlett, L. A., Stylianopoulos, T., Munn, L. L., Tearney, G. J., Fukumura, D., Jain, R. K. & Bouma, B. E. Three-dimensional microscopy of the tumor microenvironment in vivo using optical frequency domain imaging. Nature medicine 15, 1219-U1151 (2009).
45 Senarathna, J., Rege, A., Li, N. & Thakor, N. V. Laser Speckle Contrast Imaging: theory, instrumentation and applications. IEEE reviews in biomedical engineering 6, 99-110 (2013).
46 Hillman, E. M. C. & Moore, A. All-optical anatomical co-registration for molecular imaging of small animals using dynamic contrast. Nat Photonics 1, 526-530 (2007).

### Example 3 Supplemental Information

### Methods

Preparation of water-soluble and biocompatible SWNT-IRDye800 multicolor emitter. The preparation of water soluble and biocompatible SWNTs is based upon the detailed procedure described in another publication of our group with some modifications.1 In brief, raw HiPCO SWNTs (Unidym) were suspended in an aqueous solution containing 1 wt % sodium deoxycholate by 1 hour of bath sonication. This suspension was then ultracentrifuged at 300,000 g to remove the nanotube bundles and other large aggregates. The supernatant was retained after ultracentrifugation and 0.75 mg·ml-1 of DSPE-mPEG(5 kDa) (1,2-distearoyl-sn-glycero-3-phosphoethanolamine-N-[methoxy(polyethyleneglycol, 5000)], Laysan Bio) along with 0.25 mg·ml-1 of DSPE-PEG(5 kDa)-NH2 (1,2-distearoyl-sn-glycero-3-phosphoethanolamine-N-[amino(polyethyleneglycol, 5000)], Laysan Bio) was added to the supernatant. The resulting suspension was sonicated in a bath sonicator briefly for 5 min and then dialyzed in a 3500 Da membrane (Fisher) against 1× phosphate buffer saline (PBS) solution at pH 7.4 with a minimum of six bath changes and a minimum of two hours between changes of water bath. To remove aggregates formed during dialysis, the suspension was ultracentrifuged again for 30 min at 300,000 g. This surfactant-exchanged SWNT sample has lengths ranging between 100 nm and 2.0 μm with the average length of ˜500 nm. These amine-functionalized SWNTs were further conjugated with IRDye800 dye molecules according to the protocol developed in our group.2 Briefly, the as-made SWNT solution was concentrated down to ˜2 μm after removal of excess surfactant through 30-kDa centrifugal filters (Amicon) and was mixed with 0.1 mM IRDye800 NHS ester (LI-COR) dissolved in dimethylsulfoxide (DMSO). The reaction was allowed to proceed for 1 h at pH 7.4 before purification to remove excess IRDye800 by filtration through the 30-kDa filters. The as-made SWNT-IRDye800 conjugate solution was kept at 4° C. in the refrigerator and away from light to avoid photobleaching of the IRDye800 fluorescence.

Sorting of Large-Diameter Semiconducting (LS) Nanotubes.

A 1-cm diameter column filled with 20 ml of ally dextran-based size-exclusion gel (Sephacryl S-200, GE Healthcare) was used as filtration medium to perform diameter separation of HiPCO SWNTs.3 2.5 mg of raw HiPCO SWNTs (Unidym) was sonicated in water with 1 wt % sodium cholate (SC) for 1 h, followed by an ultracentrifugation at 300,000 g for 30 min to remove large bundles and aggregates. 80% of the SC-dispersed supernatant was collected and diluted with same volume of 1 wt % sodium dodecyl sulfate (SDS) solution to make a mixture of 0.5 wt % SC and 0.5 wt % SDS surfactants, before adding to the gel column. Metallic SWNTs passed the column directly, while semiconducting SWNTs were trapped. Finally, a 0.6 wt % SC/0.4 wt % SDS mixed surfactant solution was added to wash out and collect the LS nanotubes.

UV-Vis-NIR Absorption Measurements.

UV-Vis-NIR absorption spectra of water, 1% Intralipid in water and acetone, the mouse cranial bone and scalp skin were measured by a Cary 6000i UV-Vis-NIR spectrophotometer, background-corrected for contribution from any solvent. The measured range was 600-1800 nm. For water and 1% Intralipid in acetone, since there was no scattering of light by the samples, only absorption spectra were measured. For 1% Intralipid in water and the mouse tissue samples, since the UV-Vis-NIR spectrometer only collected light at the incident angle of the beam, the scattered incident light from the sample would not be collected and would thus add to the absorption spectra. Therefore the obtained absorption spectrum for any scattering sample should really be extinction spectra, which can be considered as a combination of the absorption spectrum and the scattering spectrum.

NIR Fluorescence Spectroscopy of the SWNT-IRDye800 Conjugate.

NIR fluorescence spectrum was taken using a home-built NIR spectroscopy setup in the 850-1650 nm region. The excitation light was provided by an 808-nm diode laser (RMPC lasers) at an output power of 160 mW and filtered by an 850-nm short-pass filter (Thorlabs), a 1000-nm short-pass filter (Thorlabs), an 1100-nm short-pass filter (Omega) and a 1300-nm short-pass filter (Omega). The excitation light was allowed to pass through the solution sample of SWNT-IRDye800 conjugate in a 1 mm path cuvette (Starna Cells) and the emission was collected in the transmission geometry. The excitation light was rejected using an 850-nm long-pass filter (Thorlabs) so that the fluorescence of both IRDye800 and SWNTs could be collected in the 850-1650 nm wavelength range. The emitted fluorescence from the solution sample was directed into a spectrometer (Acton SP2300i) equipped with a liquid-nitrogen-cooled InGaAs linear array detector (Princeton OMA-V). The emission spectrum was corrected after data acquisition to account for the laser excitation bleed-through, the sensitivity profile of the detector and extinction feature of the filter using the MATLAB software.

Mouse Handling, Surgery and Injection.

C57Bl/6 mice were obtained from Taconic Farms. All animal studies were approved by Stanford University's Administrative Panel on Laboratory Animal Care. Induction of middle cerebral artery occlusion (MCAO) and cerebral hypoperfusion was performed according to a previous study with minor modifications.4 Briefly, mice were anesthetized using 30% O2, 70% N2O, and 2% isoflurane before surgery. For induction of MCAO, an incision was made on the left common carotid artery (CCA) and a silicon-coated nylon filament (Doccol Co., CA) was introduced into the left CCA and threaded forward into internal carotid artery (ICA) until the tip occludes the origin of the middle cerebral artery (MCA). For induction of cerebral hypoperfusion, only the left external carotid artery (ECA) and common carotid artery (CCA) were ligated. Control, unsurgerized mice (n=3), mice with induced MCAO (n=4) and mice with cerebral hypoperfusion (n=4) were used in the study. The hair over the scalp skin was removed using Nair before tail-vein injection and imaging. All mice were initially anesthetized before imaging in a knockdown box with 2 L·min-1 O2 gas flow mixed with 3% Isoflurane. A nose cone delivered 1.5 L·min-1 O2 gas and 3% Isoflurane throughout imaging. For comparative cerebrovascular imaging in NIR-I, NIR-II and NIR-IIa regions, a solution (200 μL) of 0.43 mg·mL-1 (4.3 mg·kg-1 body weight) SWNT-IRDye800 conjugates was injected into a mouse intravenously. For dynamic imaging in the NIR-IIa region, a solution (200 μL) of 0.43 mg·mL-1 (4.3 mg·kg-1 body weight) SWNT-DSPE-mPEG without IRDye800 was injected into a mouse intravenously. The maximum SWNT concentration in the blood circulation was ˜4× lower than our previously found half maximal inhibitory concentration (IC50) of SWNTs to vascular endothelial cells.5 The blood circulation half-time of DSPE-mPEG functionalized SWNTs was ˜5 h,6 and our previous studies had shown the lack of acute or long-term toxicity of such PEGylated SWNTs in vivo.7-9 For the injection of nanotube solution, a 28 gauge syringe needle was inserted into the tail vein, allowing for bolus injection during the first frames of imaging. For steady-state imaging of the mouse head, injection was usually done ˜5 min before the mouse was transferred to the imaging stage and imaged. For dynamic imaging of the mouse head, injection was done in the dark and the InGaAs camera started recording images continuously immediately after the nanotube solution was injected into the tail vein.

NIR Fluorescence Imaging of Phantom and Mouse Brain in Different Sub-Regions.

A liquid-nitrogen-cooled, 320×256 pixel two-dimensional InGaAs array (Princeton Instruments) was used to take images in all sub-regions of NIR including the NIR-I, the NIR-II and the NIR-IIa regions. The excitation light was provided by an 808-nm diode laser (RMPC lasers) coupled to a collimator with a focal length of 4.5 mm (Thorlabs). The excitation light was filtered by an 850-nm short-pass filter and a 1000-nm short-pass filter (Thorlabs) before reaching the sample on the imaging plane. The excitation power density at the imaging plane was 140 mW·cm-2, significantly lower than the reported safe exposure limit of 329 mW·cm-2 at 808 nm.10 The emitted fluorescence was allowed to pass through different filter sets to ensure the NIR images taken in different sub-regions. For the NIR-I region, an 850-nm long-pass filter and a 900-nm short-pass filter (Thorlabs) were used the confine the NIR-I region of 850-900 nm. For the NIR-II region, a 910-nm long-pass filter and a 1000-nm long-pass filter (Thorlabs) were used to confine the NIR-II region of 1000-1700 nm. The upper bound at 1700 nm was determined by the sensitivity profile of the InGaAs detector. For the NIR-IIa region, a 1000-nm long-pass filter, a 1300-nm long-pass filter (Thorlabs) and a 1400-nm short-pass filter (Edmund Optics) were used to confine the NIR-II region of 1300-1400 nm. A lens pair consisting of two achromats (200 mm and 75 mm, Thorlabs) was used to focus the image onto the detector with a field of view of 25 mm×20 mm, which covered a segment of the SWNT-IRDye800-filled capillary tube in the phantom imaging case and only the head and the neck area of the mouse in the in vivo brain imaging case. A higher magnification was also achieved by using two other NIR achromats (150 mm and 200 mm, Thorlabs) to zoom into a smaller region of the brain. Phantom NIR fluorescence images were flat-field corrected to compensate for the non-uniformity of the excitation laser beam.

High Resolution Microscopic Imaging of Cerebral Vessels in the NIR-IIa Region.

High-magnification intravital imaging of cerebral vessels was carried out in epifluorescence mode with an 808-nm diode laser (RMPC lasers, 160 mW) as the excitation source and two objective lens (4× and 10×, Bausch & Lomb) for microscopic imaging. The mouse with scalp hair removed was intravenously injected with a solution (200 μL) of 0.22 mg·mL-1 (2.2 mg·kg-1 body weight) separated LS nanotubes and placed in a home-made stereotactic platform fixed on a motorized 3D translational stage (Newport) that allowed for the digital position adjustment and readout of the mouse relative to the objective. The stereotactic stage had two posts fixed on two dovetail linear translational stages (Thorlabs) allowing for fine adjustment to fix the motion of the mouse head. The resulting NIR photoluminescence was collected using the same 2D InGaAs camera as aforementioned. The emitted fluorescence was filtered through a 1000-nm long-pass filter, a 1300-nm long-pass filter (Thorlabs) and a 1400-nm short-pass filter (Edmund Optics) to ensure only photons in the 1300-1400 nm NIR-IIa region were collected. Exposure times of 0.3-1 s were used for best signal-to-noise ratio. The depth of vessels was determined by first focusing the imaging plane onto the surface of the scalp skin, setting this as zero depth, and recording the axial distance the platform had travelled relative to the objective from the zero depth to reach a vascular image, based on the digital readout of the 3D translational stage. Since the objective was immersed in air, the raw axial movement of the objective was corrected to account for the refractive index difference of scalp skin (n=1.38),11 cranial bone (n=1.56)12 and brain tissue (n=1.35)13 and to obtain the actual imaging depth inside the brain based on Snell's law.14

Dynamic Cerebral Vascular Imaging in the NIR-IIa Window.

The dynamic imaging setup was the same as the aforementioned NIR-IIa fluorescence brain imaging with lower magnification that covered the entire mouse head. The InGaAs camera was set to expose continuously, and fluorescence images in the NIR-IIa window were acquired with LabVIEW software. The exposure time for each image acquisition was 100 ms. There was also an 87.5-ms overhead time in the readout, leading to a total time of 187.5 ms between consecutive frames and a frame rate of 5.3 frames·s-1 for the video. Depending on the orientation of the mouse head, the time-course images were rotated to make the head upright in the field of view using the MATLAB built-in function imrotate. This turned the left and right cerebral hemispheres symmetrical in the image and made it easier to select ROIs for cerebral blood perfusion measurement later. Then dynamic-contrast-enhanced images were obtained by loading 100 consecutive frames starting from the frame when signal first appeared in the brain, into an array using the MATLAB software, and the built-in princomp function was used to perform PCA.5,6,15 The principal components featuring pixels showing up earlier in the video were automatically combined and used to represent arterial features while those featuring pixels showing up later in the video were combined and used to represent venous features based on the haemodynamic difference of arterial and venous flows.

Brain Blood Perfusion Measurement Using NIR-IIa Fluorescence.

In a typical procedure for brain blood perfusion measurement, consecutive NIR-IIa images taken from dynamic imaging were loaded into MATLAB software, and ROIs of both the surgerized left cerebral hemisphere and the untreated right cerebral hemisphere were selected in the lateral sulcus region. The NIR-IIa fluorescence intensity increase within each ROI was plotted as a function of time from 0 s to 3.94 s (22 frames) p.i. The plot featured a linear rising edge starting at ˜2 s followed by a plateau region due to blood saturation of NIR-IIa contrast agent. Then the plot was normalized against the saturation level of the control cerebral hemisphere (i.e., the right hemisphere) and the linear rising edge after normalization was fitted into a line with its slope proportional to blood velocity as proved in previous publications.5,16 By such analysis two slope values were obtained for each mouse, one derived from the control right cerebral hemisphere, and the other derived from the surgerized left cerebral hemisphere. Then the slope derived from the left hemisphere was normalized against that from right hemisphere to obtain the relative perfusion in the surgerized brain tissue to reveal the degree of occlusion of blood perfusion:

$$Relative Perfusion = \frac{slope of increase in the left hemisphere ( surgerized )}{slope of increase in the right hemisphere ( control )}$$

This measurement was repeated for 3-4 mice in each group to obtain statistically significant data.

Vessel Width Analysis for NIR-IIa Images.

To analyze the widths of blood vessels for in vivo cerebral vascular imaging, all images were loaded into the ImageJ software, and a line was drawn perpendicular to a linear feature of interest (i.e., a blood vessel). Then the NIR-IIa intensity values on this line were extracted and plotted against their physical locations along this line. The blood vessels intersected by the line were represented as peaks in the intensity profile and each peak was fitted into a Gaussian function using the Origin software.

Laser Doppler Flowmetry for Cerebral Blood Perfusion Measurement.

Mice were anesthetized with 1.5% isoflurane (1.5%), 02 (30%) and N20 (70%) mixture. Body temperature was maintained at 36-37° C. A flexible fiberoptic probe was affixed to the skull over the MCA (2 mm posterior and 6.5 mm lateral to bregma) after the scalp skin was removed for cerebral blood flow (CBF) measurements by laser Doppler flowmetry (LDF). Baseline CBF values were measured before vascular intervention and considered to be 100% flow.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

We claim at least the following:

1. A composition, comprising:
a water-soluble NIR-II fluorescent agent including a NIR-II emitting fluorophore and includes one or more hydrophilic polymers, wherein the NIR-II emitting fluorophore emits light in the range of 1.0 to 1.7 μm under light excitation, wherein the NIR-II emitting fluorophore is a small organic molecule.

2. The composition of claim 1, wherein the small organic molecules selected from the group consisting of: a polymethine dye that emits at about 1.0 to 1.7 μm, a cyanine dye that emits at about 1.0 to 1.7 μm, and a combination thereof.

3. The composition of claim 1, wherein the hydrophilic polymer is selected from the group consisting of: poly(acrylic acid) (PAA), poly(vinyl alcohol) (PVA), polyacrylamide, polyethylene glycol (PEG), and a combination thereof.

4. The composition of claim 1, wherein the water-soluble NIR-II fluorescent agent is functionalized with a surfactant.

5. The composition of claim 4, wherein the surfactant is selected from the group consisting of: a linear or branched a polyethylene glycol (PEG) based compound, a phospholipid-polyethylene glycol compound, n-MEG, poly(vinyl alcohol) (PVA), poly(acrylic acid) (PAA), poly(propylene furmarate-co-ethylenee glycol) (P(PF-co-EG)), polyacrylamide, polypeptides, poly-N-substituted glycine oligomers (polypeptoids), and a combination thereof.

6. The composition of claim 1, further comprising a nanotube, wherein the nanotube was separated from other nanotubes based on diameter so that the nanotube emits light in the range of 1.0 to 1.7 μm under light excitation.

7. The composition of claim 6, wherein the NIR-II emitting fluorophore is disposed within the nanotube.

8. The composition of claim 7, wherein the nanotube is a single walled carbon nanotube.

9. The composition of claim 1, wherein the water-soluble NIR-II fluorescent agent has a diameter of about 3 nm to 20 nm.

10. The composition of claim 1, further comprising: a targeting moiety, wherein the targeting moiety has an affinity for a target.

11. The composition of claim 1, wherein the NIR-II fluorescent agent has a hydrodynamic size smaller than 5.5 nm.

12. The composition of claim 1, wherein the NIR-II fluorescent agent has the characteristic of being able to be excreted through the renal system.

13. The composition of claim 1, wherein the NIR-II emitting fluorophore emits light in the range of 1.3 to 1.4 μm under light excitation.

14. The composition of claim 1, wherein the NIR-II emitting fluorophore emits light in the range of 1.5 to 1.7 μm under light excitation.

15. A composition, comprising:
a water-soluble NIR-II fluorescent agent including a NIR-II emitting fluorophore and includes one or more hydrophilic polymers, wherein the NIR-II emitting fluorophore emits light in the range of 1.0 to 1.7 μm under light excitation, wherein the NIR-II emitting fluorophore is selected from the group consisting of: a polymethine dye that emits at about 1.0 to 1.7 μm, a cyanine dye that emits at about 1.0 to 1.7 μm, and a combination thereof, and wherein the hydrophilic polymer is selected from the group consisting of: poly(acrylic acid) (PAA), poly(vinyl alcohol) (PVA), polyacrylamide, polyethylene glycol (PEG), and a combination thereof.

16. The composition of claim 15, further comprising a nanotube, wherein the nanotube was separated from other nanotubes based on diameter so that the nanotube emits light in the range of 1.0 to 1.7 μm under light excitation, and wherein the NIR-II emitting fluorophore is disposed within the nanotube.

17. The composition of claim 15, wherein the water-soluble NIR-II fluorescent agent has a diameter of about 3 nm to 20 nm.

18. The composition of claim 15, wherein the NIR-II fluorescent agent has a hydrodynamic size smaller than 5.5 nm.

19. The composition of claim 15, wherein the NIR-II emitting fluorophore emits light in the range of 1.3 to 1.4 μm under light excitation.

20. The composition of claim 15, wherein the NIR-II emitting fluorophore emits light in the range of 1.5 to 1.7 μm under light excitation.
